(19) United States
(12) Patent Application Publication
(10) Pub. No.: US 2024/0117047 A1
(43) Pub. Date: Apr. 11, 2024

(54) MONOCLONAL ANTIBODIES SPECIFIC FOR HUMAN ROR1

(71) Applicant: The Regents of the University of California, Oakland, CA (US)

(72) Inventors: Thomas J. KIPPS, San Diego, CA (US); Charles E. PRUSSAK, San Diego, CA (US); George F. WIDHOPF, II, San Diego, CA (US)

(21) Appl. No.: 18/546,081

(22) PCT Filed: Feb. 11, 2022
(86) PCT No.: PCT/US2022/016218

§ 371 Date: Aug. 10, 2023

Related U.S. Application Data
(60) Provisional application No. 63/148,567, filed on Feb. 11, 2021.

(51) Int. Cl. C07K 16/28; A61K 47/68; A61P 35/00; G01N 33/68

(52) U.S. Cl. CPC C07K 16/2803; A61K 47/6849; A61P 35/00; G01N 33/6845; G01N 33/6854; C07K 2317/24; C07K 2317/622; G01N 2333/70567

(57) ABSTRACT

Provided herein are, inter alia, antibodies (e.g. humanized antibodies, monoclonal antibodies, antibody fragments (e.g., scFvs) and antibody compositions (e.g., chimeric antigen receptors, bispecific antibodies), which bind human tyrosine kinase-like orphan receptor 1 (ROR1) with high efficiency and specificity. The antibodies and antibody compositions provided herein include novel light and heavy chain domain CDRs and framework regions and are, inter alia, useful for diagnosing and treating cancer and other ROR1-related diseases.

### RELATED APPLICATION DATA

This application claims the benefit of priority under 35 U.S.C. § 119(e) of the U.S. Patent Application No. 63/148,567, filed on Feb. 11, 2021, which is hereby incorporated by reference in its enterity and for all purposes.

### STATEMENT AS TO RIGHTS TO INVENTIONS MADE UNDER FEDERALLY SPONSORED RESEARCH AND DEVELOPMENT

This invention was made with government support under CA081534 and CA049870 awarded by the National Institutes of Health. The government has certain rights in the invention.

### SEQUENCE LISTING

The material in the accompanying Sequence Listing is hereby incorporated by reference in its entirety. The accompanying file, named “048537-641001WO_SL_ST25.txt” was created on Feb. 10, 2022 and is 92,657 bytes. The file can be accessed using Microsoft Word on a computer that uses Windows OS.

### BACKGROUND

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise. Receptor tyrosine kinases (RTKs) play critical roles in cell differentiation, proliferation, migration, angiogenesis, and survival. The receptor tyrosine kinase-like orphan receptor 1 (ROR1) is an evolutionarily-conserved type I membrane protein that belongs to the ROR subfamily and has extracellular domains that contain immunoglobulin (Ig)-like, Frizzled, and Kringle domains. ROR1-deficient mice display a variety of phenotypic defects within the skeletal and urogenital systems, as well as postnatal growth retardation. ROR1 is expressed during embryogenesis and by a variety of different cancers, but not by normal post-partum tissues, and can be considered an onco-embryonic surface antigen. Functional data suggest that ROR1 may function in non-canonical WNT-signaling to promote the survival of malignant cells. More recent studies have shown that non-canonical WNT signaling plays a major role in basal-like and other subtypes of breast cancer metastasis. Expression of ROR1 human breast cancer is also associated with activation of the AKT-CREB pathway and enhanced tumor-cell growth.

Receptor-tyrosine kinase like orphan receptor 1 (ROR1) is a conserved embryonic protein whose expression becomes progressively reduced during embryonic development in mammals. The intact protein, including its extracellular domain, does not appear to be significantly expressed in normal, adult mammal tissues. In particular, studies have not identified significant expression of ROR1 on the cell surface of normal adult human tissues, including normal, non-cancerous B cells (Baker et al., Clin. Cancer Res., 14:396 (2008); DaneshManesh et al., Int. J. Cancer, 123:1190 (2008) and Fukuda et al., Proc. Nat'l. Acad. Sci. USA, 105:3047 (2008)). However, ROR1 is expressed on the cell surface of malignant B-cells (B-CLL) and mantle cell lymphoma (MCL). It has also been reported that ROR1 is expressed in certain other cancer cell lines including Burkett's lymphoma, renal cell carcinoma, colon cancer and breast cancer (U.S. Patent Application 2007/02075110). Therefore, ROR1 can be considered a selective marker for these cancers.

### BRIEF SUMMARY

In an aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:36, a CDR H2 as set forth in SEQ ID NO:38, and a CDR H3 as set forth in SEQ ID NO:40; and wherein the light chain variable domain includes: a CDR L1 as set forth in SEQ ID NO:43, a CDR L2 as set forth in SEQ ID NO:45 and a CDR L3 as set forth in SEQ ID NO:47.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:64, a CDR H2 as set forth in SEQ ID NO:66, and a CDR H3 as set forth in SEQ ID NO:68; and wherein the light chain variable domain includes: a CDR L1 as set forth in SEQ ID NO:71, a CDR L2 as set forth in SEQ ID NO:73 and a CDR L3 as set forth in SEQ ID NO:75.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:78, a CDR H2 as set forth in SEQ ID NO:80, and a CDR H3 as set forth in SEQ ID NO:82; and wherein the light chain variable domain includes: a CDR L1 as set forth in SEQ ID NO:85, a CDR L2 as set forth in SEQ ID NO:87 and a CDR L3 as set forth in SEQ ID NO:89.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:92, a CDR H2 as set forth in SEQ ID NO:94, and a CDR H3 as set forth in SEQ ID NO:96; and wherein the light chain variable domain includes: a CDR L1 as set forth in SEQ ID NO:99, a CDR L2 as set forth in SEQ ID NO:101 and a CDR L3 as set forth in SEQ ID NO:103.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO: 106, a CDR H2 as set forth in SEQ ID NO: 108, and a CDR H3 as set forth in SEQ ID NO: 110; and wherein the light chain variable domain includes: a CDR L1 as set forth in SEQ ID NO: 113, a CDR L2 as set forth in SEQ ID NO: 115 and a CDR L3 as set forth in SEQ ID NO: 117.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:50, a CDR H2 as set forth in SEQ ID NO:52, and a CDR H3 as set forth in SEQ ID NO:54; and wherein the light chain variable domain includes: a CDR L1 as set forth in SEQ ID NO:57, a CDR L2 as set forth in SEQ ID NO:59 and a CDR L3 as set forth in SEQ ID NO:61.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO: 120, a CDR H2 as set forth in SEQ ID NO: 122, and a CDR H3 as set forth in SEQ ID NO:124; and wherein the light chain variable domain includes: a CDR L1 as set forth in SEQ ID NO: 127, a CDR L2 as set forth in SEQ ID NO:129 and a CDR L3 as set forth in SEQ ID NO:131.

In another aspect is provided an anti-ROR1 antibody, wherein the anti-ROR1 antibody binds the same epitope as an antibody including: a heavy chain variable domain including a CDR H1 as set forth in SEQ ID NO:36, a CDR H2 as set forth in SEQ ID NO:38 and a CDR H3 as set forth in SEQ ID NO:40; and a light chain variable domain including a CDR L1 as set forth in SEQ ID NO:43, a CDR L2 as set forth in SEQ ID NO:45, and a CDR L3 as set forth in SEQ ID NO:47.

In another aspect is provided an anti-ROR1 antibody, wherein the anti-ROR1 antibody binds the same epitope as an antibody including: a heavy chain variable domain including a CDR H1 as set forth in SEQ ID NO:64, a CDR H2 as set forth in SEQ ID NO:66 and a CDR H3 as set forth in SEQ ID NO:68; and a light chain variable domain including a CDR L1 as set forth in SEQ ID NO:71, a CDR L2 as set forth in SEQ ID NO:73, and a CDR L3 as set forth in SEQ ID NO: 75.

In another aspect is provided an anti-ROR1 antibody, wherein the anti-ROR1 antibody binds the same epitope as an antibody including: a heavy chain variable domain including a CDR H1 as set forth in SEQ ID NO:78, a CDR H2 as set forth in SEQ ID NO:80 and a CDR H3 as set forth in SEQ ID NO:82; and a light chain variable domain including a CDR L1 as set forth in SEQ ID NO:85, a CDR L2 as set forth in SEQ ID NO:87, and a CDR L3 as set forth in SEQ ID NO: 89.

In another aspect is provided an anti-ROR1 antibody, wherein the anti-ROR1 antibody binds the same epitope as an antibody including: a heavy chain variable domain including a CDR H1 as set forth in SEQ ID NO:92, a CDR H2 as set forth in SEQ ID NO:94 and a CDR H3 as set forth in SEQ ID NO:96; and a light chain variable domain including a CDR L1 as set forth in SEQ ID NO:99, a CDR L2 as set forth in SEQ ID NO:101, and a CDR L3 as set forth in SEQ ID NO: 103.

In another aspect is provided an anti-ROR1 antibody, wherein the anti-ROR1 antibody binds the same epitope as an antibody including: a heavy chain variable domain including a CDR H1 as set forth in SEQ ID NO:106, a CDR H2 as set forth in SEQ ID NO:108 and a CDR H3 as set forth in SEQ ID NO:110; and a light chain variable domain including a CDR L1 as set forth in SEQ ID NO: 113, a CDR L2 as set forth in SEQ ID NO:115, and a CDR L3 as set forth in SEQ ID NO: 117.

In another aspect is provided an anti-ROR1 antibody, wherein the anti-ROR1 antibody binds the same epitope as an antibody including: a heavy chain variable domain including a CDR H1 as set forth in SEQ ID NO:50, a CDR H2 as set forth in SEQ ID NO:52 and a CDR H3 as set forth in SEQ ID NO:54; and a light chain variable domain including a CDR L1 as set forth in SEQ ID NO:57, a CDR L2 as set forth in SEQ ID NO:59, and a CDR L3 as set forth in SEQ ID NO:61.

In another aspect is provided an anti-ROR1 antibody, wherein the anti-ROR1 antibody binds the same epitope as an antibody including: a heavy chain variable domain including a CDR H1 as set forth in SEQ ID NO:120, a CDR H2 as set forth in SEQ ID NO:122 and a CDR H3 as set forth in SEQ ID NO:124; and a light chain variable domain including a CDR L1 as set forth in SEQ ID NO: 127, a CDR L2 as set forth in SEQ ID NO:129, and a CDR L3 as set forth in SEQ ID NO:131.

In another aspect is provided a cell including an anti-ROR1 antibody as provided herein including embodiments thereof.

In another aspect is provided a nucleic acid encoding an antibody as provided herein including embodiments thereof.

In another aspect is provided a pharmaceutical composition including a therapeutically effective amount of an anti-ROR1 antibody as provided herein including embodiments thereof, and a pharmaceutically acceptable excipient.

In another aspect is provided a method of treating cancer in a subject in need thereof, the method including administering to a subject a therapeutically effective amount of an anti-ROR1 antibody as provided herein including embodiments thereof.

In another aspect is provided a method of identifying an anti-ROR1 antibody, the method including: (i) contacting an antibody with a first ROR1 polypeptide including a threonine at a position corresponding to position 346 of SEQ ID NO:30; (ii) detecting the antibody binding to the first ROR1 polypeptide; (iii) contacting the antibody with a second ROR 1 polypeptide not including a threonine at a position corresponding to position 346 of SEQ ID NO:30; and (iv) detecting the antibody not binding to the second ROR1 polypeptide, thereby identifying an anti-ROR1 antibody.

### BRIEF DESCRIPTION OF THE DRAWINGS

FIG. 1 shows comparison of the extracellular domains of human ROR1 (hROR1; SEQ ID NO:30) with mouse ROR1 (mROR1; SEQ ID NO:31). The name of the protein represented by the amino acid sequence is on the left margin. Amino acids are indicated by the single-letter amino acid code. The numbers provided on the right margin or above the sequences are the numbers for the position of the amino acid residue below. A dot in the sequence of mROR1 indicates sequence homology with hROR1 at that position. A letter indicates the amino acid of mROR1 that differs from that present in hROR1 at that position. The various subdomains of the ROR1 extracellular domain are indicated above the amino acid sequence, which is underlined for each subdomain. The subdomains corresponding to immunoglobulin-like domain (Ig-like domain; SEQ ID NO:32), the Cysteine-Rich Domain (CRD), and the Kringle subdomain (SEQ ID NO: 34) are indicated in bold font.

FIG. 2 shows comparison of the extracellular domain of hROR1 (SEQ ID NO:30) with chimeric human-mROR1 (#5, #14, #6, and #13) used to map the domain of hROR1 bound by each of the mAbs in this disclosure. The name of the protein represented by the amino acid sequence is on the left margin. Amino acids are indicated by the single-letter amino acid code. The numbers provided on the right margin or above the sequences are the numbers for the position of the amino acid residue below. A dot in the sequence of mROR1 indicates sequence homology with hROR1 at that position. A letter indicates the amino acid of the chimeric human-mouse ROR1 that differs from that present in hROR1 at that position. The various subdomains of the ROR1 extracellular domain are indicated above the amino acid sequence, which is underlined for each subdomain. The subdomains corresponding to immunoglobulin-like domain (Ig-like domain; SEQ ID NO:32), the Cysteine-Rich Domain (CRD), and the Kringle subdomain (SEQ ID NO: 34) are indicated in bold font.

FIG. 3 shows comparison of the extracellular domain of hROR1 (SEQ ID NO:30) with mutant forms of hROR1 used to map the epitope of hROR1 bound by each of the mAbs in this disclosure. The name of the protein represented by the amino acid sequence is on the left margin. Amino acids are indicated by the single-letter amino acid code. The numbers provided on the right margin or above the sequences are the numbers for the position of the amino acid residue below. A dot in the sequence of mROR1 indicates sequence homology with hROR1 at that position. A letter indicates the amino acid of the mutant ROR1 that differs from that present in hROR1 at that position. The various subdomains of the ROR1 extracellular domain are indicated above the amino acid sequence, which is underlined for each subdomain. The subdomain corresponding to immunoglobulin-like domain (Ig-like domain; SEQ ID NO:32), the Cysteine-Rich Domain (CRD), and the Kringle subdomain (SEQ ID NO:34) are indicated in bold font.

FIG. 4 shows representative binding of mAbs in this disclosure to hROR1, mROR1, or chimeric human-mROR1. Schematics of the chimeric constructs of the extracellular portion of ROR1 used to map the domain of hROR1 bound by each of the mAb in this disclosure. The dark regions of each construct indicate hROR1, light portions denote regions of mROR1, and hatched portions indicate deleted regions. Arrows mark the boundaries between the mouse and hROR1 regions for constructs #5, #6, #13 and #14, which depict the first 160 amino acids of the extracellular region that includes the Ig-like domain. The amino acids that differ between human and mROR1 are indicated by the single letter abbreviation at that position. (B) Each of these recombinant proteins were separated in non-denaturing polyacrylamide gel, transferred onto nylon, probed with the anti-hROR1 mAb indicated on the left, and detected with an anti-mouse IgG antibody conjugated with horse radish peroxidase. The UC-101, UC-102, UC-103, UC-104, and UC-105 mAb bind to ROR1 recombinant proteins that contain the human Ig-like domain. The UC-106 and UC-107 mAb bind to ROR1 recombinant proteins within that include the human Kringle domain. None of the antibody bind to mROR1.

FIG. 5 shows epitope mapping of the antibodies that bind within the Ig-Like domain of hROR1 (SEQ ID NO:33). None of the antibody react with mROR1 protein. The mouse or hROR1 protein have different amino acid residues at amino acid positions 138, 142, or 160; the hROR1 protein has amino acid residues E, S, or Y, at these positions, whereas the mROR1 protein has amino acid residues K, T, or S at amino acid positions 138, 142, or 160, respectively. We generated recombinant hROR1 proteins having either the mouse or human amino acid residue at these positions only, or in tandem. These recombinant proteins were separated in non-denaturing polyacrylamide gel and then transferred onto nylon, which was probed with each anti-hROR1 mAb, as indicated on the left. UC-105, UC-961, and UC-104 mAb detect recombinant proteins 2, 4, 5, and 8, but not 1, 3, 6 or 7, which are described in the legend below. Note that substitution of the human amino acid residue E at position 138 of the hROR1 protein with the mouse amino acid residue T at position 138 abrogates binding of the UC-105, UC-961, and UC-104 mAb.

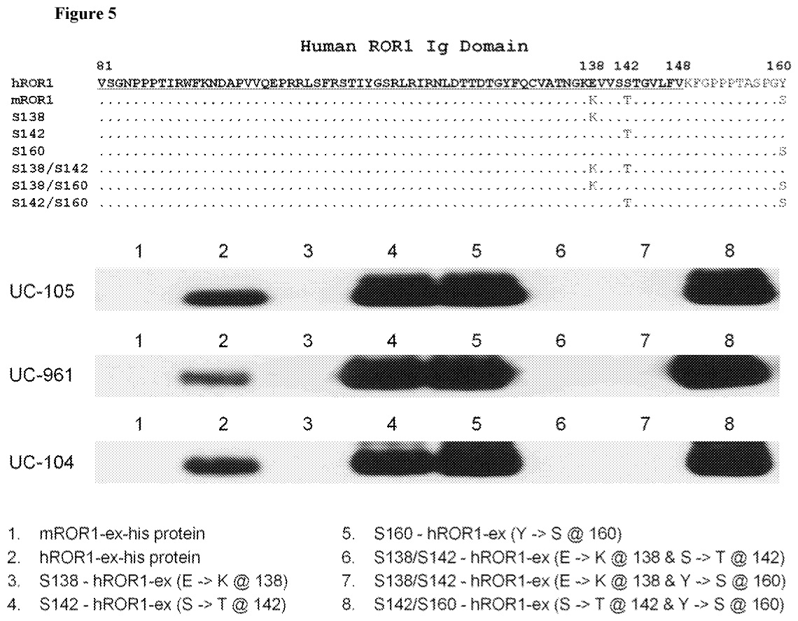

FIG. 6 shows epitope mapping of the antibodies that bind within the Kringle domain of hROR1 (SEQ ID NO:34). Neither of the antibody reacts with mROR1 protein. The mouse or hROR1 protein have a different amino acid residue at position 346 located within the kringle domain; the hROR1 protein has amino acid residue T at this position, whereas the mROR1 protein has amino acid residue S, respectively. We generated recombinant hROR1 proteins having either the mouse or human amino acid residue at this position only. These recombinant proteins were separated in non-denaturing polyacrylamide gel and then transferred onto nylon, which was probed with the UC-106 anti-hROR1 mAb, as indicated on the left. UC-106 detects the hROR1 extracellular recombinant protein, but not the hROR1 extracellular recombinant protein with only the S residue of mROR1 substituted at position 346.

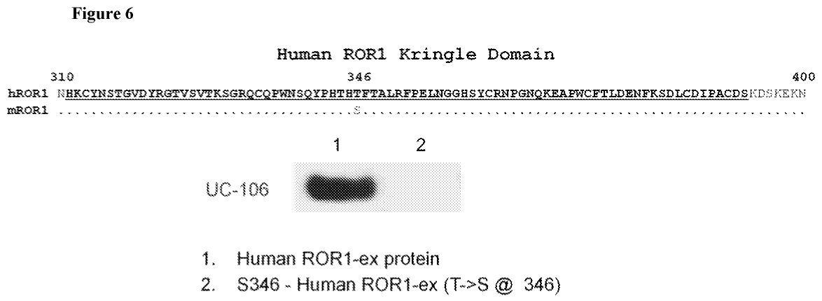

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. A nonspecific sequence as provided herein may be a sequence including scrambled nucleotides bound to each other, wherein the sequence does not have a biological function (is unfunctional).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., ROR-1) in other proteins with different numbering systems. For example, by performing a simple sequence alignment with a protein (e.g., ROR-1) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Nat. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). In human, two types of light chain are known: kappa chain (VK or Vκ), encoded by the immunoglobulin kappa locus on chromosome 2, and the lambda chain (Vλ), encoded by the immunoglobulin lambda locus on chromosome 22. Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

The term “ROR1 protein” or “ROR1” as used herein includes any of the recombinant or naturally-occurring forms of Receptor tyrosine kinase-like orphan receptor 1, also known as Tyrosine-protein kinase transmembrane receptor ROR1, Neurotrophic tyrosine kinase receptor-related 1, or variants or homologs thereof that maintain ROR1 activity (e.g. within at least 50%, 80%, 90%, 95%, 96%, 97%, 98%, 99% or 100% activity compared to ROR1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ROR1 protein. In embodiments, the ROR1 protein is substantially identical to the protein identified by SEQ ID NO:29. In embodiments, the ROR1 protein is substantially identical to the protein identified by the UniProt reference number Q01973 or a variant or homolog having substantial identity thereto. In embodiments, the ROR1 protein is substantially identical to the protein identified by SEQ ID NO:30. In embodiments, the ROR1 protein is substantially identical to the protein identified by SEQ ID NO:31.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. ROR1 protein). Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. ROR1 protein) relative to the activity or function of the protein in the absence of the inhibitor. In embodiments inhibition means negatively affecting (e.g. decreasing) the concentration or levels of ROR1 relative to the concentration or level of the protein in the absence of the inhibitor. In embodiments inhibition refers to reduction of a disease or symptoms of disease. In embodiments, inhibition refers to a reduction in the activity of ROR1. Thus, inhibition includes, at least in part, partially or totally blocking stimulation, decreasing, preventing, or delaying activation, or inactivating, desensitizing, or down-regulating signal transduction or enzymatic activity or the amount of ROR1. In embodiments, inhibition refers to a reduction of activity of ROR1 resulting from a direct interaction (e.g. an inhibitor binds to ROR1). In embodiments, inhibition refers to a reduction of activity of ROR1 from an indirect interaction (e.g. an inhibitor binds to a protein that activates ROR1, thereby preventing target protein activation).

Thus, the terms “inhibitor,” “repressor” or “antagonist” or “downregulator” interchangeably refer to a substance capable of detectably decreasing the expression or activity of a given gene or protein (e.g. ROR1 protein). The antagonist can decrease ROR1 expression or activity 10%, 20%, 30%, 40%, 50%, 60%, 70%, 80%, 90% or more in comparison to a control in the absence of the antagonist. In certain instances, ROR1 expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer, prostate cancer, pancreatic cancer, brain cancer, liver cancer, gastric cancer or a sarcoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g. a protein associated disease, a cancer associated with ROR1 activity, ROR1 associated cancer, ROR1 associated disease (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease. For example, a cancer associated with ROR1 activity or function or a ROR1 associated disease (e.g., cancer, inflammatory disease, autoimmune disease, or infectious disease), may be treated with a ROR1 modulator or ROR1 inhibitor, in the instance where increased ROR1 activity or function (e.g. signaling pathway activity) causes the disease (e.g., cancer, inflammatory disease, autoimmune disease, or infectious disease). For example, an inflammatory disease associated with ROR1 activity or function or an ROR1 associated inflammatory disease, may be treated with an ROR1 modulator or ROR1 inhibitor, in the instance where increased ROR1 activity or function (e.g. signaling pathway activity) causes the disease.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therpaeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, anticancer agents herein may include epigenetic inhibitors and multi-kinase inhibit “Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Anti-Ror1 Antibodies

Provided herein are, inter alia, antibodies (e.g., humanized antibodies, monoclonal antibodies), antibody fragments (e.g., scFvs) and antibody compositions (e.g., chimeric antigen receptors, bispecific antibodies), which bind human tyrosine kinase-like orphan receptor 1 (ROR1) with high efficiency and specificity. The antibodies and antibody compositions provided herein include novel light and heavy chain domains and have been identified to bind extracellular domains of human ROR1. For example, the antibodies provided herein including embodiments thereof, may bind the Kringle or the Ig-like domain of ROR1 with high affinity and specificity. Further, Applicants have characterized the amino acid residues in the ROR1 extracellular domains, which are important for binding of antibodies as described herein including embodiments thereof. Antibodies specifically binding the epitope described herein including embodiments thereof, are useful for binding human ROR1 with high effectivity and affinity and inhibiting ROR1 signaling in cells expressing ROR1. The antibodies provided herein including embodiments thereof, may be used for diagnostic and therapeutic purposes in cancer and other ROR1 related diseases. The variable light chain and the variable heavy chain domains provided herein may, inter alia, form part of an anti-ROR1 chimeric antigen receptor or an anti-ROR1 bispecific antibody. Furthermore, due to their internalization properties, some of the anti-ROR1 antibodies provided herein may be attached to therapeutic moieties and used as antibody-drug conjugates (ADC), or they may be attached to a detectable moiety and used for diagnostic purposes. The antibodies provided herein including embodiments thereof, have an ability to inhibit migration of ROR1 expressing metastatic cells and therefore are capable of mitigating the risk of metastasis in patients with ROR1-expressing cancer cells

In an aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:36, a CDR H2 as set forth in SEQ ID NO:38, and a CDR H3 as set forth in SEQ ID NO:40; and wherein the light chain variable domain includes: a CDR L1 as set forth in SEQ ID NO:43, a CDR L2 as set forth in SEQ ID NO:45 and a CDR L3 as set forth in SEQ ID NO:47.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:64, a CDR H2 as set forth in SEQ ID NO:66, and a CDR H3 as set forth in SEQ ID NO:68; and wherein the light chain variable domain includes: a CDR L1 as set forth in SEQ ID NO:71, a CDR L2 as set forth in SEQ ID NO:73 and a CDR L3 as set forth in SEQ ID NO:75.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:78, a CDR H2 as set forth in SEQ ID NO:80, and a CDR H3 as set forth in SEQ ID NO:82; and wherein the light chain variable domain includes: a CDR L1 as set forth in SEQ ID NO:85, a CDR L2 as set forth in SEQ ID NO:87 and a CDR L3 as set forth in SEQ ID NO:89.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:92, a CDR H2 as set forth in SEQ ID NO:94, and a CDR H3 as set forth in SEQ ID NO:96; and wherein the light chain variable domain includes: a CDR L1 as set forth in SEQ ID NO:99, a CDR L2 as set forth in SEQ ID NO:101 and a CDR L3 as set forth in SEQ ID NO:103.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO: 106, a CDR H2 as set forth in SEQ ID NO: 108, and a CDR H3 as set forth in SEQ ID NO: 110; and wherein the light chain variable domain includes: a CDR L1 as set forth in SEQ ID NO: 113, a CDR L2 as set forth in SEQ ID NO: 115 and a CDR L3 as set forth in SEQ ID NO: 117.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO:50, a CDR H2 as set forth in SEQ ID NO:52, and a CDR H3 as set forth in SEQ ID NO:54; and wherein the light chain variable domain includes: a CDR L1 as set forth in SEQ ID NO:57, a CDR L2 as set forth in SEQ ID NO:59 and a CDR L3 as set forth in SEQ ID NO:61.

In another aspect is provided an anti-tyrosine kinase-like orphan receptor 1 (ROR1) antibody including a heavy chain variable domain and a light chain variable domain, wherein the heavy chain variable domain includes a CDR H1 as set forth in SEQ ID NO: 120, a CDR H2 as set forth in SEQ ID NO: 122, and a CDR H3 as set forth in SEQ ID NO: 124; and wherein the light chain variable domain includes: a CDR L1 as set forth in SEQ ID NO:127, a CDR L2 as set forth in SEQ ID NO:129 and a CDR L3 as set forth in SEQ ID NO:131.

As described above, a “heavy chain variable (VH) domain” as provided herein refers to the variable region of the heavy chain of an antibody, an antibody variant or fragment thereof. Likewise, the “light chain variable (VL) domain” as provided herein refers to the variable region of the light chain of an antibody, an antibody variant or fragment thereof. Variable kappa light chain (VK) and VL as referred to herein are used interchangeably and refer to the variable light chain domain of an antibody, an antibody variant or fragment thereof. The heavy chain variable domain and light chain variable domain together form the paratope, which binds an antigen (epitope). The paratope or antigen-binding site is formed at the N-terminus of an antibody, an antibody variant or fragment thereof. In embodiments, the heavy chain variable (VH) domain includes CDR H1, CDR H2, CDR H3 and FR H1, FR H2, FR H3, FR H4 (framework regions) of an antibody heavy chain. In embodiments, the heavy chain variable (VH) domain and a heavy chain constant (CH1) domain form part of an antibody heavy chain. In embodiments, the heacy chain variable (VH) domain and one or more heacy chain constant (CH1, CH2, or CH3) domains form part of an antibody heavy chain. In embodiments, the light chain variable (VL) domain and a light chain constant (CL) domain form part of an antibody light chain. Thus, in embodiments, the heavy chain variable (VH) domain forms part of an antibody. In embodiments, the light chain variable (VL) domain forms part of an antibody. In embodiments, the heavy chain variable domain (VH) forms part of a therapeutic antibody. In embodiments, the light chain variable domain (VL) forms part of a therapeutic antibody. In embodiments, the heavy chain variable domain (VH) forms part of a human antibody. In embodiments, the light chain variable domain (VL) forms part of a human antibody. In embodiments, the heavy chain variable domain (VH) forms part of a humanized antibody. In embodiments, the light chain variable domain (VL) forms part of a humanized antibody. In embodiments, the heavy chain variable domain (VH) forms part of a chimeric antibody. In embodiments, the light chain variable domain (VL) forms part of a chimeric antibody. In embodiments, the heavy chain variable domain (VH) forms part of an antibody fragment. In embodiments, the light chain variable domain (VL) forms part of an antibody fragment. In embodiments, the heavy chain variable domain (VH) forms part of an antibody variant. In embodiments, the light chain variable domain (VL) forms part of an antibody variant. In embodiments, the heavy chain variable domain (VH) forms part of a Fab. In embodiments, the light chain variable domain (VL) forms part of a Fab. In embodiments, the heavy chain variable domain (VH) forms part of a scFv. In embodiments, the light chain variable domain (VL) forms part of a scFv.

In embodiments, the antibody is a humanized antibody, a chimeric antibody, a Fab′ fragment or a scFv. In embodiments, the antibody is a humanized antibody. In embodiments, the antibody is a chimeric antibody. In embodiments, the antibody is a Fab′ fragment. In embodiments, the antibody is a scFv.

In embodiments, the heavy chain variable domain includes the sequence of SEQ ID NO:2, SEQ ID NO:6, SEQ ID NO:10, SEQ ID NO:14, SEQ ID NO:18, SEQ ID NO:22 or SEQ ID NO:26. In embodiments, the heavy chain variable domain includes the sequence of SEQ ID NO:2. In embodiments, the heavy chain variable domain includes the sequence of SEQ ID NO:6. In embodiments, the heavy chain variable domain includes the sequence of SEQ ID NO:10. In embodiments, the heavy chain variable domain includes the sequence of SEQ ID NO:14. In embodiments, the heavy chain variable domain includes the sequence of SEQ ID NO:18. In embodiments, the heavy chain variable domain includes the sequence of SEQ ID NO:22. In embodiments, the heavy chain variable domain includes the sequence of SEQ ID NO:26.

In embodiments, the light chain variable domain includes the sequence of SEQ ID NO:4, SEQ ID NO:8, SEQ ID NO:12, SEQ ID NO:16, SEQ ID NO:20, SEQ ID NO:24 or SEQ ID NO:28. In embodiments, the light chain variable domain includes the sequence of SEQ ID NO:4. In embodiments, the light chain variable domain includes the sequence of SEQ ID NO:8. In embodiments, the light chain variable domain includes the sequence of SEQ ID NO:12. In embodiments, the light chain variable domain includes the sequence of SEQ ID NO:16. In embodiments, the light chain variable domain includes the sequence of SEQ ID NO:20. In embodiments, the light chain variable domain includes the sequence of SEQ ID NO:24. In embodiments, the light chain variable domain includes the sequence of SEQ ID NO:28.

In embodiments, the heavy chain variable domain includes the amino acid sequence of SEQ ID NO:2. In embodiments, the heavy chain variable domain is the amino acid sequence of SEQ ID NO:2. In embodiments, the heavy chain variable domain includes a FR H1 as set forth in SEQ ID NO:35, a FR H2 as set forth in SEQ ID NO:37, a FR H3 as set forth in SEQ ID NO:39 and a FR H4 as set forth in SEQ ID NO:41.

In embodiments, the heavy chain variable domain includes the amino acid sequence of SEQ ID NO: 6. In embodiments, the heavy chain variable domain is the amino acid sequence of SEQ ID NO:6. In embodiments, the heavy chain variable domain includes a FR H1 as set forth in SEQ ID NO:63, a FR H2 as set forth in SEQ ID NO: 65, a FR H3 as set forth in SEQ ID NO:67 and a FR H4 as set forth in SEQ ID NO: 69.

In embodiments, the heavy chain variable domain includes the amino acid sequence of SEQ ID NO: 10. In embodiments, the heavy chain variable domain is the amino acid sequence of SEQ ID NO: 10. In embodiments, the heavy chain variable domain includes a FR H1 as set forth in SEQ ID NO:77, a FR H2 as set forth in SEQ ID NO:79, a FR H3 as set forth in SEQ ID NO:81 and a FR H4 as set forth in SEQ ID NO: 83.

In embodiments, the heavy chain variable domain includes the amino acid sequence of SEQ ID NO: 14. In embodiments, the heavy chain variable domain is the amino acid sequence of SEQ ID NO: 14. In embodiments, the heavy chain variable domain includes a FR H1 as set forth in SEQ ID NO:91, a FR H2 as set forth in SEQ ID NO:93, a FR H3 as set forth in SEQ ID NO:95 and a FR H4 as set forth in SEQ ID NO: 97.

In embodiments, the heavy chain variable domain includes the amino acid sequence of SEQ ID NO:18. In embodiments, the heavy chain variable domain is the amino acid sequence of SEQ ID NO: 18. In embodiments, the heavy chain variable domain includes a FR H1 as set forth in SEQ ID NO: 105, a FR H2 as set forth in SEQ ID NO: 107, a FR H3 as set forth in SEQ ID NO:109 and a FR H4 as set forth in SEQ ID NO:111.

In embodiments, the heavy chain variable domain includes the amino acid sequence of SEQ ID NO: 22. In embodiments, the heavy chain variable domain is the amino acid sequence of SEQ ID NO:22. In embodiments, the heavy chain variable domain includes a FR H1 as set forth in SEQ ID NO:49, a FR H2 as set forth in SEQ ID NO:51, a FR H3 as set forth in SEQ ID NO:53 and a FR H4 as set forth in SEQ ID NO:55.

In embodiments, the heavy chain variable domain includes the amino acid sequence of SEQ ID NO: 26. In embodiments, the heavy chain variable domain is the amino acid sequence of SEQ ID NO:26. In embodiments, the heavy chain variable domain includes a FR H1 as set forth in SEQ ID NO:119, a FR H2 as set forth in SEQ ID NO:121, a FR H3 as set forth in SEQ ID NO:123 and a FR H4 as set forth in SEQ ID NO: 125.

In embodiments, the light chain variable domain includes the amino acid sequence of SEQ ID NO:4 In embodiments, the light chain variable domain is the amino acid sequence of SEQ ID NO:4 In embodiments, the light chain variable domain includes a FR L1 as set forth in SEQ ID NO:42, a FR L2 as set forth in SEQ ID NO:44, a FR L3 as set forth in SEQ ID NO:46 and a FR L4 as set forth in SEQ ID NO:48.

In embodiments, the light chain variable domain includes the amino acid sequence of SEQ ID NO: 8. In embodiments, the light chain variable domain is the amino acid sequence of SEQ ID NO: 8. In embodiments, the light chain variable domain includes a FR L1 as set forth in SEQ ID NO:70, a FR L2 as set forth in SEQ ID NO:72, a FR L3 as set forth in SEQ ID NO:74 and a FR L4 as set forth in SEQ ID NO:76.

In embodiments, the light chain variable domain includes the amino acid sequence of SEQ ID NO: 12. In embodiments, the light chain variable domain is the amino acid sequence of SEQ ID NO: 12. In embodiments, the light chain variable domain includes a FR L1 as set forth in SEQ ID NO:84, a FR L2 as set forth in SEQ ID NO:86, a FR L3 as set forth in SEQ ID NO:88 and a FR L4 as set forth in SEQ ID NO:90.

In embodiments, the light chain variable domain includes the amino acid sequence of SEQ ID NO:16. In embodiments, the light chain variable domain is the amino acid sequence of SEQ ID NO: 16. In embodiments, the light chain variable domain includes a FR L1 as set forth in SEQ ID NO:98, a FR L2 as set forth in SEQ ID NO:100, a FR L3 as set forth in SEQ ID NO:102 and a FR L4 as set forth in SEQ ID NO: 104.

In embodiments, the light chain variable domain includes the amino acid sequence of SEQ ID NO:20. In embodiments, the light chain variable domain is the amino acid sequence of SEQ ID NO:20. In embodiments, the light chain variable domain includes a FR L1 as set forth in SEQ ID NO:112, a FR L2 as set forth in SEQ ID NO: 114, a FR L3 as set forth in SEQ ID NO:116 and a FR L4 as set forth in SEQ ID NO: 118.

In embodiments, the light chain variable domain includes the amino acid sequence of SEQ ID NO:24. In embodiments, the light chain variable domain is the amino acid sequence of SEQ ID NO:24. In embodiments, the light chain variable domain includes a FR L1 as set forth in SEQ ID NO:56, a FR L2 as set forth in SEQ ID NO:58, a FR L3 as set forth in SEQ ID NO:60 and a FR L4 as set forth in SEQ ID NO:62.

In embodiments, the light chain variable domain includes the amino acid sequence of SEQ ID NO:28. In embodiments, the light chain variable domain is the amino acid sequence of SEQ ID NO:28. In embodiments, the light chain variable domain includes a FR L1 as set forth in SEQ ID NO:126, a FR L2 as set forth in SEQ ID NO: 128, a FR L3 as set forth in SEQ ID NO:130 and a FR L4 as set forth in SEQ ID NO: 132.

In embodiments, the heavy chain variable domain includes the amino acid sequence of SEQ ID NO:2 and the light chain variable domain includes the amino acid sequence of SEQ ID NO:4 In embodiments, the heavy chain variable domain is the amino acid sequence of SEQ ID NO:2 and the light chain variable domain is the amino acid sequence of SEQ ID NO:4.

In embodiments, the heavy chain variable domain includes the amino acid sequence of SEQ ID NO:6 and the light chain variable domain includes the amino acid sequence of SEQ ID NO:8 In embodiments, the heavy chain variable domain is the amino acid sequence of SEQ ID NO:6 and the light chain variable domain is the amino acid sequence of SEQ ID NO:8.

In embodiments, the heavy chain variable domain includes the amino acid sequence of SEQ ID NO:10 and the light chain variable domain includes the amino acid sequence of SEQ ID NO: 12. In embodiments, the heavy chain variable domain is the amino acid sequence of SEQ ID NO: 10 and the light chain variable domain is the amino acid sequence of SEQ ID NO: 12.

In embodiments, the heavy chain variable domain includes the amino acid sequence of SEQ ID NO:14 and the light chain variable domain includes the amino acid sequence of SEQ ID NO:16. In embodiments, the heavy chain variable domain is the amino acid sequence of SEQ ID NO:14 and the light chain variable domain is the amino acid sequence of SEQ ID NO:16.

In embodiments, the heavy chain variable domain includes the amino acid sequence of SEQ ID NO:18 and the light chain variable domain includes the amino acid sequence of SEQ ID NO:20. In embodiments, the heavy chain variable domain is the amino acid sequence of SEQ ID NO:18 and the light chain variable domain is the amino acid sequence of SEQ ID NO:20.

In embodiments, the heavy chain variable domain includes the amino acid sequence of SEQ ID NO:22 and the light chain variable domain includes the amino acid sequence of SEQ ID NO:24. In embodiments, the heavy chain variable domain is the amino acid sequence of SEQ ID NO:22 and the light chain variable domain is the amino acid sequence of SEQ ID NO:24.

In embodiments, the heavy chain variable domain includes the amino acid sequence of SEQ ID NO:26 and the light chain variable domain includes the amino acid sequence of SEQ ID NO:28. In embodiments, the heavy chain variable domain is the amino acid sequence of SEQ ID NO:26 and the light chain variable domain is the amino acid sequence of SEQ ID NO:28.

In embodiments, the antibody is capable of binding a ROR1 protein. In embodiments, the antibody binds a ROR1 protein. In embodiments, the ROR1 protein is a human ROR1 protein. In embodiments, the ROR1 protein includes the amino acid sequence of SEQ ID NO:29. In embodiments, the ROR1 protein is the amino acid sequence of SEQ ID NO:29. In embodiments, the ROR1 protein includes the amino acid sequence of SEQ ID NO:30. In embodiments, the ROR1 protein is the amino acid sequence of SEQ ID NO:30. In embodiments, the ROR1 protein includes the amino acid sequence of SEQ ID NO:31. In embodiments, the ROR1 protein is the amino acid sequence of SEQ ID NO:31. In embodiments, the ROR1 protein includes the amino acid sequence of SEQ ID NO:32. In embodiments, the ROR1 protein is the amino acid sequence of SEQ ID NO:32. In embodiments, the ROR1 protein includes the amino acid sequence of SEQ ID NO:34. In embodiments, the ROR1 protein is the amino acid sequence of SEQ ID NO:34.

In embodiments, the antibody is capable of binding an extracellular domain of the ROR1 protein. In embodiments, the antibody binds an extracellular domain of the ROR1 protein. In embodiments, the extracellular domain includes the amino acid sequence of SEQ ID NO:30. In embodiments, the extracellular domain is the amino acid sequence of SEQ ID NO:30. In embodiments, the extracellular domain includes the amino acid sequence of SEQ ID NO:32. In embodiments, the extracellular domain is the amino acid sequence of SEQ ID NO:32. In embodiments, the extracellular domain includes the amino acid sequence of SEQ ID NO:34. In embodiments, the extracellular domain is the amino acid sequence of SEQ ID NO:34. In embodiments, the extracellular domain is an Ig-like domain. Thus, in embodiments, the anti-ROR1 antibody binds the Ig-like domain of human ROR1. In embodiments the Ig-like domain includes the amino acid sequence of SEQ ID NO:32. In embodiments the Ig-like domain is the amino acid sequence of SEQ ID NO:32. In embodiments, the Ig-like domain includes the amino acid sequence of SEQ ID NO:33. In embodiments the Ig-like domain is the amino acid sequence of SEQ ID NO:33.

In embodiments, the extracellular domain is a Kringle domain. Thus, in embodiments, the anti-ROR1 antibody binds the Kringle domain of human ROR1. In embodiments, the Kringle domain includes the amino acid sequence of SEQ ID NO:34. In embodiments, the Kringle domain is the amino acid sequence of SEQ ID NO:34.

In embodiments, the anti-ROR1 antibody binds to a ROR1 polypeptide having the sequence of SEQ ID NO:29. In embodiments, the anti-ROR1 antibody binds to a ROR1 polypeptide having the sequence of SEQ ID NO:30. In embodiments, the anti-ROR1 antibody binds to a ROR1 polypeptide including the sequence of SEQ ID NO:29. In embodiments, the anti-ROR1 antibody binds to a ROR1 polypeptide including the sequence of SEQ ID NO:30. In embodiments, the anti-ROR1 antibody binds to a ROR1 polypeptide having the sequence of SEQ ID NO:32. In embodiments, the anti-ROR1 antibody binds to a ROR1 polypeptide having the sequence of SEQ ID NO:34. In embodiments, the anti-ROR1 antibody binds to a ROR1 polypeptide including the sequence of SEQ ID NO:32. In embodiments, the anti-ROR1 antibody binds to a ROR1 polypeptide including the sequence of SEQ ID NO:34. In embodiments, the anti-ROR1 antibody does not bind to mouse ROR1. In embodiments, the anti-ROR1 antibody does not bind to a ROR1 polypeptide having the sequence of SEQ ID NO:31.

In embodiments, the anti-ROR1 antibody binds a ROR1 polypeptide including a glutamic acid at a position corresponding to position 138 of SEQ ID NO:30. In embodiments, the anti-ROR1 antibody does not bind a ROR1 polypeptide including a lysine at a position corresponding to position 138 of SEQ ID NO:30.

In embodiments, the anti-ROR1 antibody binds a ROR1 polypeptide including a threonine at a position corresponding to position 346 of SEQ ID NO:30. In embodiments, the anti-ROR1 antibody does not bind a ROR1 polypeptide including a serine at a position corresponding to position 346 of SEQ ID NO:30.

In another aspect is provided an anti-ROR1 antibody, wherein the anti-ROR1 antibody binds the same epitope as an antibody including: a heavy chain variable domain 5 including a CDR H1 as set forth in SEQ ID NO:36, a CDR H2 as set forth in SEQ ID NO:38 and a CDR H3 as set forth in SEQ ID NO:40; and a light chain variable domain including a CDR L1 as set forth in SEQ ID NO:43, a CDR L2 as set forth in SEQ ID NO:45, and a CDR L3 as set forth in SEQ ID NO:47. In embodiments, the heavy chain variable domain includes the sequence of SEQ ID NO: 2, and the light chain variable domain includes the the amino acid sequence of SEQ ID NO:4 In embodiments, the heavy chain variable domain is the amino acid sequence of SEQ ID NO:2, and the light chain variable domain is the sequence of SEQ ID NO:4.

In another aspect is provided an anti-ROR1 antibody, wherein the anti-ROR1 antibody binds the same epitope as an antibody including: a heavy chain variable domain including a CDR H1 as set forth in SEQ ID NO:64, a CDR H2 as set forth in SEQ ID NO:66 and a CDR H3 as set forth in SEQ ID NO:68; and a light chain variable domain including a CDR L1 as set forth in SEQ ID NO:71, a CDR L2 as set forth in SEQ ID NO:73, and a CDR L3 as set forth in SEQ ID NO:75. In embodiments, the heavy chain variable domain includes the amino acid sequence of SEQ ID NO: 6, and the light chain variable domain includes the amino acid sequence of SEQ ID NO:8. In embodiments, the heavy chain variable domain is the amino acid sequence of SEQ ID NO:6, and the light chain variable domain is the amino acid sequence of SEQ ID NO: 8.

In another aspect is provided an anti-ROR1 antibody, wherein the anti-ROR1 antibody binds the same epitope as an antibody including: a heavy chain variable domain including a CDR H1 as set forth in SEQ ID NO:78, a CDR H2 as set forth in SEQ ID NO:80 and a CDR H3 as set forth in SEQ ID NO:82; and a light chain variable domain including a CDR L1 as set forth in SEQ ID NO:85, a CDR L2 as set forth in SEQ ID NO:87, and a CDR L3 as set forth in SEQ ID NO:89. In embodiments, the heavy chain variable domain includes the amino acid sequence of SEQ ID NO: 10, and the light chain variable domain includes the amino acid sequence of SEQ ID NO:12. In embodiments, the heavy chain variable domain is the amino acid sequence of SEQ ID NO: 10, and the light chain variable domain is the amino acid sequence of SEQ ID NO: 12.

In another aspect is provided an anti-ROR1 antibody, wherein the anti-ROR1 antibody binds the same epitope as an antibody including: a heavy chain variable domain including a CDR H1 as set forth in SEQ ID NO:92, a CDR H2 as set forth in SEQ ID NO:94 and a CDR H3 as set forth in SEQ ID NO:96; and a light chain variable domain including a CDR L1 as set forth in SEQ ID NO:99, a CDR L2 as set forth in SEQ ID NO:101, and a CDR L3 as set forth in SEQ ID NO:103. In embodiments, the heavy chain variable domain includes the amino acid sequence of SEQ ID NO:14, and the light chain variable domain includes the amino acid sequence of SEQ ID NO:16. In embodiments, the heavy chain variable domain is the amino acid sequence of SEQ ID NO: 14, and the light chain variable domain is the amino acid sequence of SEQ ID NO: 16.

In another aspect is provided an anti-ROR1 antibody, wherein the anti-ROR1 antibody binds the same epitope as an antibody including: a heavy chain variable domain including a CDR H1 as set forth in SEQ ID NO:106, a CDR H2 as set forth in SEQ ID NO:108 and a CDR H3 as set forth in SEQ ID NO:110; and a light chain variable domain including a CDR L1 as set forth in SEQ ID NO: 113, a CDR L2 as set forth in SEQ ID NO:115, and a CDR L3 as set forth in SEQ ID NO: 117. In embodiments, the heavy chain variable domain includes the amino acid sequence of SEQ ID NO: 18, and the light chain variable domain includes the amino acid sequence of SEQ ID NO:20. In embodiments, the heavy chain variable domain is the amino acid sequence of SEQ ID NO: 18, and the light chain variable domain is the amino acid sequence of SEQ ID NO:20.

In another aspect is provided an anti-ROR1 antibody, wherein the anti-ROR1 antibody binds the same epitope as an antibody including: a heavy chain variable domain including a CDR H1 as set forth in SEQ ID NO:50, a CDR H2 as set forth in SEQ ID NO:52 and a CDR H3 as set forth in SEQ ID NO:54; and a light chain variable domain including a CDR L1 as set forth in SEQ ID NO:57, a CDR L2 as set forth in SEQ ID NO:59, and a CDR L3 as set forth in SEQ ID NO:61. In embodiments, the heavy chain variable domain includes the amino acid sequence of SEQ ID NO: 22, and the light chain variable domain includes the amino acid sequence of SEQ ID NO:24. In embodiments, the heavy chain variable domain is the amino acid sequence of SEQ ID NO:22, and the light chain variable domain is the amino acid sequence of SEQ ID NO:24.

In another aspect is provided an anti-ROR1 antibody, wherein the anti-ROR1 antibody binds the same epitope as an antibody including: a heavy chain variable domain including a CDR H1 as set forth in SEQ ID NO:120, a CDR H2 as set forth in SEQ ID NO:122 and a CDR H3 as set forth in SEQ ID NO:124; and a light chain variable domain including a CDR L1 as set forth in SEQ ID NO: 127, a CDR L2 as set forth in SEQ ID NO:129, and a CDR L3 as set forth in SEQ ID NO:131. In embodiments, the heavy chain variable domain includes the amino acid sequence of SEQ ID NO:26, and the light chain variable domain includes the amino acid sequence of SEQ ID NO:28. In embodiments, the heavy chain variable domain is the amino acid sequence of SEQ ID NO: 26, and the light chain variable domain is the amino acid sequence of SEQ ID NO:28.

The antibodies provided herein including embodiments thereof are capable of specifically binding to human ROR1 epitope. In embodiments, the epitope is a human ROR1 protein. In embodiments, the epitope is not a mouse ROR1 protein. In embodiments, the epitope includes an Ig-like domain. In embodiments, the Ig-like domain includes the amino acid sequence of SEQ ID NO:32. In embodiments, the Ig-like domain is the amino acid sequence of SEQ ID NO:32. In embodiments, the Ig-like domain includes the amino acid sequence of SEQ ID NO:33. In embodiments, the Ig-like domain is the amino acid sequence of SEQ ID NO:33. In embodiments, the epitope includes a glutamic acid at a position corresponding to position 138 of SEQ ID NO:30. In embodiments, the epitope does not include a lysine at a position corresponding to position 138 of SEQ ID NO:30. In embodiments, the epitope includes a Kringle domain. In embodiments, the Kringle domain includes the amino acid sequene of SEQ ID NO:34. In embodiments, the Kringle domain is the amino acid sequence of SEQ ID NO:34. In embodiments, the epitope includes a threonine at a position corresponding to position 346 of SEQ ID NO:30. In embodiments, the epitope does not include a serine at a position corresponding to position 346 of SEQ ID NO:30.

In embodiments, the antibody is bound to a ROR1 protein. In embodiments, the ROR1 protein forms part of a cell. In embodiments, the ROR protein is expressed on the surface of a cell. In embodiments, the cell is a cancer cell. In embodiments, the cancer cell is a B cell leukemia cell, a mantle cell lymphoma (MCL) cell, a Burkett's Lymphoma cell, a lymphoma cell, a chronic lymphocytic leukemia (CLL) cell, an Acute Myeloid Leukemia (AML) cell, a B-Cell Acute Lymphoblastic Leukemia (B-ALL) cell, a T-cell acute lymphoblastic leukemia (T-ALL) cell, a renal cancer cell, a colon cancer cell, a breast cancer cell, an ovarian cancer cell, a lung cancer cell, a skin cancer cell, a pancreatic cancer cell, a testicular cancer cell, a bladder cancer cell, a uterine cancer cell, a prostate cancer cell, or an adrenal cancer cell. In embodiments, the cancer cell is a B cell leukemia cell. In embodiments, the cancer cell is a mantle cell lymphoma (MCL) cell. In embodiments, the cancer is a Burkett's Lymphoma cell. In embodiments, the cancer cell is a lymphoma cell. In embodiments, the cancer cell is a chronic lymphocytic leukemia (CLL) cell. In embodiments, the cancer cell is an Acute Myeloid Leukemia (AML) cell. In embodiments, the cancer cell is a B-Cell Acute Lymphoblastic Leukemia (B-ALL) cell. In embodiments, the cancer cell is a T-cell acute lymphoblastic leukemia (T-ALL) cell. In embodiments, the cancer cell is a renal cancer cell. In embodiments, the cancer cell is a colon cancer cell. In embodiments, the cancer cell is a breast cancer cell. In embodiments, the cancer cell is an ovarian cancer cell. In embodiments, the cancer cell is a lung cancer cell. In embodiments, the cancer cell is a skin cancer cell. In embodiments, the cancer cell is a pancreatic cancer cell. In embodiments, the cancer cell is a testicular cancer cell. In embodiments, the cancer cell is a bladder cancer cell. In embodiments, the cancer cell is a uterine cancer cell. In embodiments, the cancer cell is a prostate cancer cell. In embodiments, the cancer cell is an adrenal cancer cell.

In embodiments, the antibody is attached to a therapeutic agent. In embodiments, the antibody is attached to a diagnostic agent.

The ability of an antibody to bind a specific epitope (e.g., a ROR1 protein, a Kringle domain or Ig-like domain of ROR1) can be described by the equilibrium dissociation constant (K_{D}). The equilibrium dissociation constant (K_{D}) as defined herein is the ratio of the dissociation rate (K-off) and the association rate (K-on) of an antibody to a ROR1 protein. It is described by the following formula: K_{D}=K-off/K-on.

In embodiments, the antibody binds the ROR1 protein with an equilibrium dissociation constant (K_{D}) from 0.01 nM to 10 nM. In embodiments, the antibody binds the ROR1 protein with a K_{D} from 0.1 nM to 10 nM. In embodiments, the antibody binds the ROR1 protein with a K_{D} from 0.2 nM to 10 nM. In embodiments, the antibody binds the ROR1 protein with a K_{D} from 0.3 nM to 10 nM. In embodiments, the antibody binds the ROR1 protein with a K_{D} from 0.4 nM to 10 nM. In embodiments, the antibody binds the ROR1 protein with a K_{D} from 0.5 nM to 10 nM. In embodiments, the antibody binds the ROR1 protein with a K_{D} from 0.6 nM to 10 nM. In embodiments, the antibody binds the ROR1 protein with a K_{D} from 0.7 nM to 10 nM. In embodiments, the antibody binds the ROR1 protein with a K_{D} from 0.8 nM to 10 nM. In embodiments, the antibody binds the ROR1 protein with a K_{D} from 0.9 nM to 1 nM. In embodiments, the antibody binds the ROR1 protein with a K_{D} from 1 nM to 10 nM. In embodiments, the antibody binds the ROR1 protein with an equilibrium dissociation constant (K_{D}) from 1.1 nM to 10 nM. In embodiments, the antibody binds the ROR1 protein with a K_{D} from 1.2 nM to 10 nM. In embodiments, the antibody binds the ROR1 protein with a K_{D} from 1.3 nM to 10 nM. In embodiments, the antibody binds the ROR1 protein with a K_{D} from 1.4 nM to 10 nM. In embodiments, the antibody binds the ROR1 protein with a K_{D} from 1.5 nM to 10 nM. In embodiments, the antibody binds the ROR1 protein with a K_{D} from 1.6 nM to 10 nM. In embodiments, the antibody binds the ROR1 protein with a K_{D} from 1.7 nM to 10 nM. In embodiments, the antibody binds the ROR1 protein with a K_{D} from 1.8 nM to 10 nM. In embodiments, the antibody binds the ROR1 protein with a K_{D} from 1.9 nM to 10 nM. In embodiments, the antibody binds the ROR1 protein with a K_{D} from 2 nM to 10 nM. In embodiments, the antibody binds the ROR1 protein with an equilibrium dissociation constant (K_{D}) from 2.1 nM to 10 nM. In embodiments, the antibody binds the ROR1 protein with a K_{D} from 2.2 nM to 10 nM. In embodiments, the antibody binds the ROR1 protein with a K_{D} from 2.3 nM to 10 nM. In embodiments, the antibody binds the ROR1 protein with a K_{D} from 2.4 nM to 10 nM. In embodiments, the antibody binds the ROR1 protein with a K_{D} from 2.5 nM to 10 nM. In embodiments, the antibody binds the ROR1 protein with a K_{D} from 2.6 nM to 10 nM. In embodiments, the antibody binds the ROR1 protein with a K_{D} from 2.7 nM to 10 nM. In embodiments, the antibody binds the ROR1 protein with a K_{D} from 2.8 nM to 10 nM. In embodiments, the antibody binds the ROR1 protein with a K_{D} from 2.9 nM to 10 nM. In embodiments, the antibody binds the ROR1 protein with a K_{D} from 3 nM to 10 nM. In embodiments, the antibody binds the ROR1 protein with an equilibrium dissociation constant (K_{D}) from 4.1 nM to 10 nM. In embodiments, the antibody binds the ROR1 protein with a K_{D} from 4.2 nM to 10 nM. In embodiments, the antibody binds the ROR1 protein with a K_{D} from 4.3 nM to 10 nM. In embodiments, the antibody binds the ROR1 protein with a K_{D} from 4.4 nM to 10 nM. In embodiments, the antibody binds the ROR1 protein with a K_{D} from 4.5 nM to 10 nM. In embodiments, the antibody binds the ROR1 protein with a K_{D} from 4.6 nM to 10 nM. In embodiments, the antibody binds the ROR1 protein with a K_{D} from 4.7 nM to 10 nM. In embodiments, the antibody binds the ROR1 protein with a K_{D} from 4.8 nM to 10 nM. In embodiments, the antibody binds the ROR1 protein with a K_{D} from 4.9 nM to 10 nM. In embodiments, the antibody binds the ROR1 protein with a K_{D} from 5 nM to 10 nM. In embodiments, the antibody binds the ROR1 protein with an equilibrium dissociation constant (K_{D}) from 5.1 nM to 10 nM. In embodiments, the antibody binds the ROR1 protein with a K_{D} from 5.2 nM to 10 nM. In embodiments, the antibody binds the ROR1 protein with a K_{D} from 5.3 nM to 10 nM. In embodiments, the antibody binds the ROR1 protein with a K_{D} from 5.4 nM to 10 nM. In embodiments, the antibody binds the ROR1 protein with a K_{D} from 5.5 nM to 10 nM. In embodiments, the antibody binds the ROR1 protein with a K_{D} from 5.6 nM to 10 nM. In embodiments, the antibody binds the ROR1 protein with a K_{D} from 5.7 nM to 10 nM. In embodiments, the antibody binds the ROR1 protein with a K_{D} from 5.8 nM to 10 nM. In embodiments, the antibody binds the ROR1 protein with a K_{D} from 5.9 nM to 10 nM. In embodiments, the antibody binds the ROR1 protein with a K_{D} from 6 nM to 10 nM. In embodiments, the antibody binds the ROR1 protein with an equilibrium dissociation constant (K_{D}) from 6.1 nM to 10 nM. In embodiments, the antibody binds the ROR1 protein with a K_{D} from 6.2 nM to 10 nM. In embodiments, the antibody binds the ROR1 protein with a K_{D} from 6.3 nM to 10 nM. In embodiments, the antibody binds the ROR1 protein with a K_{D} from 6.4 nM to 10 nM. In embodiments, the antibody binds the ROR1 protein with a K_{D} from 6.5 nM to 10 nM. In embodiments, the antibody binds the ROR1 protein with a K_{D} from 6.6 nM to 10 nM. In embodiments, the antibody binds the ROR1 protein with a K_{D} from 6.7 nM to 10 nM. In embodiments, the antibody binds the ROR1 protein with a K_{D} from 6.8 nM to 10 nM. In embodiments, the antibody binds the ROR1 protein with a K_{D} from 6.9 nM to 10 nM. In embodiments, the antibody binds the ROR1 protein with a K_{D} from 7 nM to 10 nM. In embodiments, the antibody binds the ROR1 protein with an equilibrium dissociation constant (K_{D}) from 7.1 nM to 10 nM. In embodiments, the antibody binds the ROR1 protein with a K_{D} from 7.2 nM to 10 nM. In embodiments, the antibody binds the ROR1 protein with a K_{D} from 7.3 nM to 10 nM. In embodiments, the antibody binds the ROR1 protein with a K_{D} from 7.4 nM to 10 nM. In embodiments, the antibody binds the ROR1 protein with a K_{D} from 7.5 nM to 10 nM. In embodiments, the antibody binds the ROR1 protein with a K_{D} from 7.6 nM to 10 nM. In embodiments, the antibody binds the ROR1 protein with a K_{D} from 7.7 nM to 10 nM. In embodiments, the antibody binds the ROR1 protein with a K_{D} from 7.8 nM to 10 nM. In embodiments, the antibody binds the ROR1 protein with a K_{D} from 7.9 nM to 10 nM. In embodiments, the antibody binds the ROR1 protein with a K_{D} from 8 nM to 10 nM. In embodiments, the antibody binds the ROR1 protein with an equilibrium dissociation constant (K_{D}) from 8.1 nM to 10 nM. In embodiments, the antibody binds the ROR1 protein with a K_{D} from 8.2 nM to 10 nM. In embodiments, the antibody binds the ROR1 protein with a K_{D} from 8.3 nM to 10 nM. In embodiments, the antibody binds the ROR1 protein with a K_{D} from 8.4 nM to 10 nM. In embodiments, the antibody binds the ROR1 protein with a K_{D} from 8.5 nM to 10 nM. In embodiments, the antibody binds the ROR1 protein with a K_{D} from 8.6 nM to 10 nM. In embodiments, the antibody binds the ROR1 protein with a K_{D} from 8.7 nM to 10 nM. In embodiments, the antibody binds the ROR1 protein with a K_{D} from 8.8 nM to 10 nM. In embodiments, the antibody binds the ROR1 protein with a K_{D} from 8.9 nM to 10 nM. In embodiments, the antibody binds the ROR1 protein with a K_{D} from 9 nM to 10 nM. In embodiments, the antibody binds the ROR1 protein with an equilibrium dissociation constant (K_{D}) from 9.1 nM to 10 nM. In embodiments, the antibody binds the ROR1 protein with a K_{D} from 9.2 nM to 10 nM. In embodiments, the antibody binds the ROR1 protein with a K_{D} from 9.3 nM to 10 nM. In embodiments, the antibody binds the ROR1 protein with a K_{D} from 9.4 nM to 10 nM. In embodiments, the antibody binds the ROR1 protein with a K_{D} from 9.5 nM to 10 nM. In embodiments, the antibody binds the ROR1 protein with a K_{D} from 9.6 nM to 10 nM. In embodiments, the antibody binds the ROR1 protein with a K_{D} from 9.7 nM to 10 nM. In embodiments, the antibody binds the ROR1 protein with a K_{D} from 9.8 nM to 10 nM. In embodiments, the antibody binds the ROR1 protein with a K_{D} from 9.9 nM to 10 nM.

In embodiments, the antibody binds the ROR1 protein with a K_{D} from 0.01 nM to 9.9 nM. In embodiments, the antibody binds the ROR1 protein with a K_{D} from 0.01 nM to 9.8 nM. In embodiments, the antibody binds the ROR1 protein with a K_{D} from 0.01 nM to 9.7 nM. In embodiments, the antibody binds the ROR1 protein with a K_{D} from 0.01 nM to 9.6 nM. In embodiments, the antibody binds the ROR1 protein with a K_{D} from 0.01 nM to 9.5 nM. In embodiments, the antibody binds the ROR1 protein with a K_{D} from 0.01 nM to 9.4 nM. In embodiments, the antibody binds the ROR1 protein with a K_{D} from 0.01 nM to 9.3 nM. In embodiments, the antibody binds the ROR1 protein with a K_{D} from 0.01 nM to 9.2 nM. In embodiments, the antibody binds the ROR1 protein with a K_{D} from 0.01 nM to 9.1 nM. In embodiments, the antibody binds the ROR1 protein with a K_{D} from 0.01 nM to 9 nM. In embodiments, the antibody binds the ROR1 protein with a K_{D} from 0.01 nM to 8.9 nM. In embodiments, the antibody binds the ROR1 protein with a K_{D} from 0.01 nM to 8.8 nM. In embodiments, the antibody binds the ROR1 protein with a K_{D} from 0.01 nM to 8.7 nM. In embodiments, the antibody binds the ROR1 protein with a K_{D} from 0.01 nM to 8.6 nM. In embodiments, the antibody binds the ROR1 protein with a K_{D} from 0.01 nM to 8.5 nM. In embodiments, the antibody binds the ROR1 protein with a K_{D} from 0.01 nM to 8.4 nM. In embodiments, the antibody binds the ROR1 protein with a K_{D} from 0.01 nM to 8.3 nM. In embodiments, the antibody binds the ROR1 protein with a K_{D} from 0.01 nM to 8.2 nM. In embodiments, the antibody binds the ROR1 protein with a K_{D} from 0.01 nM to 8.1 nM. In embodiments, the antibody binds the ROR1 protein with a K_{D} from 0.01 nM to 8 nM. In embodiments, the antibody binds the ROR1 protein with a K_{D} from 0.01 nM to 7.9 nM. In embodiments, the antibody binds the ROR1 protein with a K_{D} from 0.01 nM to 7.8 nM. In embodiments, the antibody binds the ROR1 protein with a K_{D} from 0.01 nM to 7.7 nM. In embodiments, the antibody binds the ROR1 protein with a K_{D} from 0.01 nM to 7.6 nM. In embodiments, the antibody binds the ROR1 protein with a K_{D} from 0.01 nM to 7.5 nM. In embodiments, the antibody binds the ROR1 protein with a K_{D} from 0.01 nM to 7.4 nM. In embodiments, the antibody binds the ROR1 protein with a K_{D} from 0.01 nM to 7.3 nM. In embodiments, the antibody binds the ROR1 protein with a K_{D} from 0.01 nM to 7.2 nM. In embodiments, the antibody binds the ROR1 protein with a K_{D} from 0.01 nM to 7.1 nM. In embodiments, the antibody binds the ROR1 protein with a K_{D} from 0.01 nM to 7 nM. In embodiments, the antibody binds the ROR1 protein with a K_{D} from 0.01 nM to 6.9 nM. In embodiments, the antibody binds the ROR1 protein with a K_{D} from 0.01 nM to 6.8 nM. In embodiments, the antibody binds the ROR1 protein with a K_{D} from 0.01 nM to 6.7 nM. In embodiments, the antibody binds the ROR1 protein with a K_{D} from 0.01 nM to 6.6 nM. In embodiments, the antibody binds the ROR1 protein with a K_{D} from 0.01 nM to 6.5 nM. In embodiments, the antibody binds the ROR1 protein with a K_{D} from 0.01 nM to 6.4 nM. In embodiments, the antibody binds the ROR1 protein with a K_{D} from 0.01 nM to 6.3 nM. In embodiments, the antibody binds the ROR1 protein with a K_{D} from 0.01 nM to 6.2 nM. In embodiments, the antibody binds the ROR1 protein with a K_{D} from 0.01 nM to 6.1 nM. In embodiments, the antibody binds the ROR1 protein with a K_{D} from 0.01 nM to 6 nM. In embodiments, the antibody binds the ROR1 protein with a K_{D} from 0.01 nM to 5.9 nM. In embodiments, the antibody binds the ROR1 protein with a K_{D} from 0.01 nM to 5.8 nM. In embodiments, the antibody binds the ROR1 protein with a K_{D} from 0.01 nM to 5.7 nM. In embodiments, the antibody binds the ROR1 protein with a K_{D} from 0.01 nM to 5.6 nM. In embodiments, the antibody binds the ROR1 protein with a K_{D} from 0.01 nM to 5.5 nM. In embodiments, the antibody binds the ROR1 protein with a K_{D} from 0.01 nM to 5.4 nM. In embodiments, the antibody binds the ROR1 protein with a K_{D} from 0.01 nM to 5.3 nM. In embodiments, the antibody binds the ROR1 protein with a K_{D} from 0.01 nM to 5.2 nM. In embodiments, the antibody binds the ROR1 protein with a K_{D} from 0.01 nM to 5.1 nM. In embodiments, the antibody binds the ROR1 protein with a K_{D} from 0.01 nM to 5 nM. In embodiments, the antibody binds the ROR1 protein with a K_{D} from 0.01 nM to 4.9 nM. In embodiments, the antibody binds the ROR1 protein with a K_{D} from 0.01 nM to 4.8 nM. In embodiments, the antibody binds the ROR1 protein with a K_{D} from 0.01 nM to 4.7 nM. In embodiments, the antibody binds the ROR1 protein with a K_{D} from 0.01 nM to 4.6 nM. In embodiments, the antibody binds the ROR1 protein with a K_{D} from 0.01 nM to 4.5 nM. In embodiments, the antibody binds the ROR1 protein with a K_{D} from 0.01 nM to 4.4 nM. In embodiments, the antibody binds the ROR1 protein with a K_{D} from 0.01 nM to 4.3 nM. In embodiments, the antibody binds the ROR1 protein with a K_{D} from 0.01 nM to 4.2 nM. In embodiments, the antibody binds the ROR1 protein with a K_{D} from 0.01 nM to 4.1 nM. In embodiments, the antibody binds the ROR1 protein with a K_{D} from 0.01 nM to 4 nM. In embodiments, the antibody binds the ROR1 protein with a K_{D} from 0.01 nM to 3.9 nM. In embodiments, the antibody binds the ROR1 protein with a K_{D} from 0.01 nM to 3.8 nM. In embodiments, the antibody binds the ROR1 protein with a K_{D} from 0.01 nM to 3.7 nM. In embodiments, the antibody binds the ROR1 protein with a K_{D} from 0.01 nM to 3.6 nM. In embodiments, the antibody binds the ROR1 protein with a K_{D} from 0.01 nM to 3.5 nM. In embodiments, the antibody binds the ROR1 protein with a K_{D} from 0.01 nM to 3.4 nM. In embodiments, the antibody binds the ROR1 protein with a K_{D} from 0.01 nM to 3.3 nM. In embodiments, the antibody binds the ROR1 protein with a K_{D} from 0.01 nM to 3.2 nM. In embodiments, the antibody binds the ROR1 protein with a K_{D} from 0.01 nM to 3.1 nM. In embodiments, the antibody binds the ROR1 protein with a K_{D} from 0.01 nM to 3 nM. In embodiments, the antibody binds the ROR1 protein with a K_{D} from 0.01 nM to 2.9 nM. In embodiments, the antibody binds the ROR1 protein with a K_{D} from 0.01 nM to 2.8 nM. In embodiments, the antibody binds the ROR1 protein with a K_{D} from 0.01 nM to 2.7 nM. In embodiments, the antibody binds the ROR1 protein with a K_{D} from 0.01 nM to 2.6 nM. In embodiments, the antibody binds the ROR1 protein with a K_{D} from 0.01 nM to 2.5 nM. In embodiments, the antibody binds the ROR1 protein with a K_{D} from 0.01 nM to 2.4 nM. In embodiments, the antibody binds the ROR1 protein with a K_{D} from 0.01 nM to 2.3 nM. In embodiments, the antibody binds the ROR1 protein with a K_{D} from 0.01 nM to 2.2 nM. In embodiments, the antibody binds the ROR1 protein with a K_{D} from 0.01 nM to 2.1 nM. In embodiments, the antibody binds the ROR1 protein with a K_{D} from 0.01 nM to 2 nM. In embodiments, the antibody binds the ROR1 protein with a K_{D} from 0.01 nM to 1.9 nM. In embodiments, the antibody binds the ROR1 protein with a K_{D} from 0.01 nM to 1.8 nM. In embodiments, the antibody binds the ROR1 protein with a K_{D} from 0.01 nM to 1.7 nM. In embodiments, the antibody binds the ROR1 protein with a K_{D} from 0.01 nM to 1.6 nM. In embodiments, the antibody binds the ROR1 protein with a K_{D} from 0.01 nM to 1.5 nM. In embodiments, the antibody binds the ROR1 protein with a K_{D} from 0.01 nM to 1.4 nM. In embodiments, the antibody binds the ROR1 protein with a K_{D} from 0.01 nM to 1.3 nM. In embodiments, the antibody binds the ROR1 protein with a K_{D} from 0.01 nM to 1.2 nM. In embodiments, the antibody binds the ROR1 protein with a K_{D} from 0.01 nM to 1.1 nM. In embodiments, the antibody binds the ROR1 protein with a K_{D} from 0.01 nM to 0.9 nM. In embodiments, the antibody binds the ROR1 protein with a K_{D} from 0.01 nM to 0.8 nM. In embodiments, the antibody binds the ROR1 protein with a K_{D} from 0.01 nM to 0.7 nM. In embodiments, the antibody binds the ROR1 protein with a K_{D} from 0.01 nM to 0.6 nM. In embodiments, the antibody binds the ROR1 protein with a K_{D} from 0.01 nM to 0.5 nM. In embodiments, the antibody binds the ROR1 protein with a K_{D} from 0.01 nM to 0.4 nM. In embodiments, the antibody binds the ROR1 protein with a K_{D} from 0.01 nM to 0.3 nM. In embodiments, the antibody binds the ROR1 protein with a K_{D} from 0.01 nM to 0.2 nM. In embodiments, the antibody binds the ROR1 protein with a K_{D} from 0.01 nM to 0.1 nM.

In embodiments, the antibody binds the ROR1 protein with a a K_{D} of 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 nM.

In embodiments, the antibody binds the ROR1 protein with a K_{D} of about 0.06 nM. In embodiments, the antibody is antibody 6E6 and binds a ROR1 protein with a K_{D} of 0.01 nM to 10 nM. In embodiments, the antibody 6E6 binds a ROR1 protein with a K_{D} of 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 nM. In embodiments, the antibody is antibody 6E6 and binds a ROR1 protein with a K_{D} of 0.06 nM.

### Chimeric Antigen Receptor Proteins

As described above, the heavy chain variable (VH) domain and the light chain variable (VL) domain provided herein including embodiments thereof, may each independently form part of an antibody, a fragment of an antibody, or a a chimeric antigen receptor or bispecific antibody. Provided herein are, inter alia, chimeric antigen receptors and bispecific antibodies, which include the light chain variable (VL) domain and/or the heavy chain variable (VH) domain as provided herein and are therefore capable of binding human ROR1 effectively and efficiently. The antibody region of the chimeric antigen receptor may include any of the light chain and heavy chain variable domains provided herein including embodiments thereof. The light chain variable (VL) domain and/or the heavy chain variable (VH) domain as provided herein may form part of a chimeric antigen receptor.

An “antibody region” as provided herein refers to a monovalent or multivalent protein moiety that forms part of the recombinant protein (e.g., CAR, bispecific antibody) provided herein including embodiments thereof. A person of ordinary skill in the art will therefore immediately recognize that the antibody region is a protein moiety capable of binding an antigen (epitope). Thus, the antibody region provided herein may include a domain of an antibody (e.g., a light chain variable (VL) domain, a heavy chain variable (VH) domain) or a fragment of an antibody (e.g., Fab). In embodiments, the antibody region is a protein conjugate. A “protein conjugate” as provided herein refers to a construct consisting of more than one polypeptide, wherein the polypeptides are bound together covalently or non-covalently. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker. In embodiments, the antibody region is an scFv. The antibody region may include a light chain variable (VL) domain and/or a heavy chain variable (VH) domain. In embodiments, the antibody region includes a light chain variable (VL) domain. In embodiments, the antibody region includes a heavy chain variable (VH) domain.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4 or CD3-zeta. In embodiments, the transmembrane domain is a CD4 transmembrane domain.

In embodiments, the transmembrane domain is a CD28 transmembrane domain. The term “CD28 transmembrane domain” as provided herein includes any of the recombinant or naturally-occurring forms of the transmembrane domain of CD28, or variants or homologs thereof that maintain CD28 transmembrane domain activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, CD28 is the protein as identified by the NCBI sequence reference GI:340545506, homolog or functional fragment thereof.

In embodiments, the transmembrane domain is a CD8 transmembrane domain. The term “CD8 transmembrane domain” as provided herein includes any of the recombinant or naturally-occurring forms of the transmembrane domain of CD8, or variants or homologs thereof that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, CD8 is the protein as identified by the NCBI sequence reference GI:225007534, homolog or functional fragment thereof.

In embodiments, the transmembrane domain is a CD4 transmembrane domain. The term “CD4 transmembrane domain” as provided herein includes any of the recombinant or naturally-occurring forms of the transmembrane domain of CD4, or variants or homologs thereof that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, CD4 is the protein as identified by the NCBI sequence reference GI:303522473, homolog or functional fragment thereof.

In embodiments, the transmembrane domain is a CD3-zeta (also known as CD247) transmembrane domain. The term “CD3-zeta transmembrane domain” as provided herein includes any of the recombinant or naturally-occurring forms of the transmembrane domain of CD3-zeta, or variants or homologs thereof that maintain CD3-zeta transmembrane domain activity (e.g. within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In embodiments, CD3-zeta is the protein as identified by the NCBI sequence reference GI:166362721, homolog or functional fragment thereof.

In embodiments, the chimeric antigen receptor further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4, PD-1, CD28, CD69). In embodiments, the intracellular T-cell signaling domain is a CD3 ζ intracellular T-cell signaling domain.

In embodiments, the chimeric antigen receptor further includes an intracellular co-stimulatory T-cell signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a 4-1BB intracellular co-stimulatory signaling domain. In embodiments, the intracellular co-stimulatory signaling domain is an ICOS intracellular co-stimulatory signaling domain. In embodiments, the intracellular co-stimulatory signaling domain is an OX-40 intracellular co-stimulatory signaling domain.

In embodiments, the antibody region includes an Fc domain. In embodiments, the antibody region includes a spacer region. In embodiments, the spacer region is between the transmembrane domain and the antibody region. A “spacer region” as provided herein is a polypeptide connecting the antibody region with the transmembrane domain. In embodiments, the spacer region connects the heavy chain constant region with the transmembrane domain. In embodiments, the spacer region includes an Fc region. In embodiments, the spacer region is an Fc region. Examples of spacer regions contemplated for the compositions provided herein include without limitation, immunoglobulin molecules or fragments thereof (e.g., IgG1, IgG2, IgG3, IgG4) and immunoglobulin molecules or fragments thereof (e.g., IgG1, IgG2, IgG3, IgG4) including mutations affecting Fc receptor binding. In embodiments, the spacer region is a hinge region.

The term “CTLA-4” as referred to herein includes any of the recombinant or naturally-occurring forms of the cytotoxic T-lymphocyte-associated protein 4 protein, also known as CD152 (cluster of differentiation 152), or variants or homologs thereof that maintain CTLA-4 activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, the CTLA-4 protein is substantially identical to the protein identified by the UniProt reference number P16410 or a variant or homolog having substantial identity thereto.

The term “CD28” as referred to herein includes any of the recombinant or naturally-occurring forms of the Cluster of Differentiation 28 protein, or variants or homologs thereof that maintain CD28 activity (e.g. within at least 50%, 80%, 90%, 95%, 96%, 97%, 98%, 99% or 100% activity compared to CD28). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD69” as referred to herein includes any of the recombinant or naturally-occurring forms of the Cluster of Differentiation 69 protein, or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “4-1BB” as referred to herein includes any of the recombinant or naturally-occurring forms of the 4-1BB protein, also known as tumor necrosis factor receptor superfamily member 9 (TNFRSF9), Cluster of Differentiation 137 (CD137) and induced by lymphocyte activation (ILA), or variants or homologs thereof that maintain 4-1BB activity (e.g. within at least 50%, 80%, 90%, 95%, 96%, 97%, 98%, 99% or 100% activity compared to 4-1BB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the 4-1BB protein is substantially identical to the protein identified by the UniProt reference number Q07011 or a variant or homolog having substantial identity thereto.

The chimeric antigen receptors provided herein may include any of the anti-ROR1 antibodies or fragments thereof described herein.

### Bispecific Antibodies

The light chain variable (VL) domain and the heavy chain variable (VH) domain as provided herein may form part of a bispecific antibody. Thus, the second antibody region may include any of the light chain and/or heavy chain variable domains provided herein including embodiments thereof.

The term “effector cell ligand” as provided herein refers to a cell surface molecule expressed on an effector cell of the immune system (e.g., a cytotoxic T cell, a helper T cell, a B cell, a natural killer cell). Upon binding of the first antibody region to the effector cell ligand expressed on the effector cell, the effector cell is activated and able to exert its function (e.g., selective killing or eradication of malignant, infected or otherwise unhealthy cells). In embodiments, the effector cell ligand is a CD3 protein. In embodiments, the effector cell ligand is a CD16 protein. In embodiments, the effector cell ligand is a CD32 protein. In embodiments, the effector cell ligand is a NKp46 protein. The first antibody region as provided herein may be an antibody, an antibody variant, a fragment of an antibody or a fragment of an antibody variant.

A “CD3 protein” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex.

A “CD16 protein” as referred to herein includes any of the recombinant or naturally-occurring forms of the Cluster of Differentiation 16 (CD16) protein, also known as low affinity immunoglobulin gamma Fc region receptor III-A, or variants or homologs thereof that maintain CD16 activity (e.g. within at least 50%, 80%, 90%, 95%, 96%, 97%, 98%, 99% or 100% activity compared to CD16). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD16 protein. In embodiments, the CD16 protein is substantially identical to the protein identified by the UniProt reference number P08637 or a variant or homolog having substantial identity thereto.

A “CD32 protein” as referred to herein includes any of the recombinant or naturally-occurring forms of the Cluster of Differentiation 32 (CD32) protein, also known as low affinity immunoglobulin gamma Fc region receptor II-A, or variants or homologs thereof that maintain CD32 activity (e.g. within at least 50%, 80%, 90%, 95%, 96%, 97%, 98%, 99% or 100% activity compared to CD32). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD32 protein. In embodiments, the CD32 protein is substantially identical to the protein identified by the UniProt reference number P12318 or a variant or homolog having substantial identity thereto.

A “NKp46 protein” as referred to herein includes any of the recombinant or naturally-occurring forms of the NKp46 protein, also known as natural cytotoxicity triggering receptor 1, or variants or homologs thereof that maintain NKp46 activity (e.g. within at least 50%, 80%, 90%, 95%, 96%, 97%, 98%, 99% or 100% activity compared to NKp46). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring NKp46 protein. In embodiments, the NKp46 protein is substantially identical to the protein identified by the UniProt reference number 076036 or a variant or homolog having substantial identity thereto.

The bispecific antibody provided herein may include any of the ROR1 antibodies or fragments thereof described herein.

In embodiments, the first antibody region is a first Fab′ fragment or the second antibody region is a second Fab′ fragment. In embodiments, the first antibody region is a single chain variable fragment (scFv) or the second antibody region is a second single chain variable fragment (scFv).

In embodiments, the first antibody region is a first Fab′ fragment or the second antibody region is a second Fab′ fragment. In embodiments, the first antibody region is a single chain variable fragment (scFv) or the second antibody region is a second single chain variable fragment (scFv).

In embodiments, the scFv of the second antibody region binds the ROR1 protein with a K_{D} from 0.01 nM to 150 nM. In embodiments, the scFv of the second antibody region binds the ROR1 protein with a K_{D} from 0.1 nM to 150 nM. In embodiments, the scFv of the second antibody region binds the ROR1 protein with a K_{D} from 1 nM to 150 nM. In embodiments, the scFv of the second antibody region binds the ROR1 protein with a K_{D} from 10 nM to 150 nM. In embodiments, the scFv of the second antibody region binds the ROR1 protein with a K_{D} from 20 nM to 150 nM. In embodiments, the scFv of the second antibody region binds the ROR1 protein with a K_{D} from 30 nM to 150 nM. In embodiments, the scFv of the second antibody region binds the ROR1 protein with a K_{D} from 40 nM to 150 nM. In embodiments, the scFv of the second antibody region binds the ROR1 protein with a K_{D} from 50 nM to 150 nM. In embodiments, the scFv of the second antibody region binds the ROR1 protein with a K_{D} from 60 nM to 150 nM. In embodiments, the scFv of the second antibody region binds the ROR1 protein with a K_{D} from 70 nM to 150 nM. In embodiments, the scFv of the second antibody region binds the ROR1 protein with a K_{D} from 80 nM to 150 nM. In embodiments, the scFv of the second antibody region binds the ROR1 protein with a K_{D} from 90 nM to 150 nM. In embodiments, the scFv of the second antibody region binds the ROR1 protein with a K_{D} from 100 nM to 150 nM. In embodiments, the scFv of the second antibody region binds the ROR1 protein with a K_{D} from 110 nM to 150 nM. In embodiments, the scFv of the second antibody region binds the ROR1 protein with a K_{D} from 120 nM to 150 nM. In embodiments, the scFv of the second antibody region binds the ROR1 protein with a K_{D} from 130 nM to 150 nM. In embodiments, the scFv of the second antibody region binds the ROR1 protein with a K_{D} from 140 nM to 150 nM.

In embodiments, the scFv of the second antibody region binds the ROR1 protein with a K_{D} from 0.01 nM to 140 nM. In embodiments, the scFv of the second antibody region binds the ROR1 protein with a K_{D} from 0.01 nM to 130 nM. In embodiments, the scFv of the second antibody region binds the ROR1 protein with a K_{D} from 0.01 nM to 120 nM. In embodiments, the scFv of the second antibody region binds the ROR1 protein with a K_{D} from 0.01 nM to 110 nM. In embodiments, the scFv of the second antibody region binds the ROR1 protein with a K_{D} from 0.01 nM to 100 nM. In embodiments, the scFv of the second antibody region binds the ROR1 protein with a K_{D} from 0.01 nM to 90 nM. In embodiments, the scFv of the second antibody region binds the ROR1 protein with a K_{D} from 0.01 nM to 80 nM. In embodiments, the scFv of the second antibody region binds the ROR1 protein with a K_{D} from 0.01 nM to 70 nM. In embodiments, the scFv of the second antibody region binds the ROR1 protein with a K_{D} from 0.01 nM to 60 nM. In embodiments, the scFv of the second antibody region binds the ROR1 protein with a K_{D} from 0.01 nM to 50 nM. In embodiments, the scFv of the second antibody region binds the ROR1 protein with a K_{D} from 0.01 nM to 40 nM. In embodiments, the scFv of the second antibody region binds the ROR1 protein with a K_{D} from 0.01 nM to 30 nM. In embodiments, the scFv of the second antibody region binds the ROR1 protein with a K_{D} from 0.01 nM to 20 nM. In embodiments, the scFv of the second antibody region binds the ROR1 protein with a K_{D} from 0.01 nM to 10 nM. In embodiments, the scFv of the second antibody region binds the ROR1 protein with a K_{D} from 0.01 nM to 1 nM.

In embodiments, the scFv of the second antibody region binds the ROR1 protein with a K_{D} of about 0.07 nM. In embodiments, the scFv of the second antibody region binds the ROR1 protein with a K_{D} of 0.07 nM. In embodiments, the scFv of the second antibody region is the 6E6 scFv and binds the ROR1 protein with a K_{D} of 0.07 nM.

The second antibody region may include a light chain variable (VL) domain or a heavy chain variable (VH) domain. In embodiments, the second antibody region includes a light chain variable (VL) domain. In embodiments, the second antibody region includes a heavy chain variable (VH) domain.

In embodiments, the second antibody region is bound to a ROR1 protein. In embodiments, the ROR1 protein is expressed on a cell. In embodiments, the cell is a cancer cell. In embodiments, the cancer cell is cancer is a breast cancer cell, ovarian cancer cell, pancreatic cancer cell, cervical cancer cell, gastric cancer cell, renal cancer cell, head and neck cancer cell, bone cancer cell, skin cancer cell or prostate cancer cell. In embodiments, the cancer cell is a breast cancer cell. In embodiments, the cancer cell is an ovarian cancer cell. In embodiments, the cancer cell is a pancreatic cancer cell. In embodiments, the cancer cell is cancer is a cervical cancer cell. In embodiments, the cancer cell is cancer is a gastric cancer cell. In embodiments, the cancer cell is cancer is a renal cancer cell. In embodiments, the cancer cell is cancer is a head and neck cancer cell. In embodiments, the cancer cell is a cancer is bone cancer cell. In embodiments, the cancer cell is cancer is a skin cancer cell. In embodiments, the cancer cell is cancer is a prostate cancer cell.

### Cellular Compositions

The compositions provided herein include cellular compositions including an antibody as provided herein including embodiments thereof. Thus, in an aspect is provided a cell comprising an anti-ROR1 antibody provided herein including embodiments therof.

### Nucleic Acid Compositions

The compositions provided herein include nucleic acid molecules encoding the anti-ROR1 antibodies, CARs and bispecific antibodies or portions thereof provided herein including embodiments thereof. The antibodies, CARs and bispecific antibodies encoded by the isolated nucleic acid are described in detail throughout this application (including the description above and in the examples section). Thus, in an aspect, an isolated nucleic acid encoding an antibody as provided herein including embodiments thereof is provided.

### Pharmaceutical Compositions

The compositions provided herein include pharmaceutical compositions including the anti-ROR1 antibodies, CARs and bispecific antibodies provided herein including embodiments thereof. Thus, in an aspect is provided a pharmaceutical composition including a therapeutically effective amount of an antibody as provided herein including embodiments thereof and a pharmaceutically acceptable excipient.

In another aspect is provided a pharmaceutical composition including a therapeutically effective amount of a CAR as provided herein including embodiments thereof and a pharmaceutically acceptable excipient.

In another aspect is provided a pharmaceutical composition including a therapeutically effective amount of a bispecific antibody as provided herein including embodiments thereof and a pharmaceutically acceptable excipient.

### Methods

The compositions (e.g., the anti-ROR1 antibodies, CARs and bispecific antibodies) provided herein, including embodiments thereof, are contemplated as providing effective treatments for diseases such as cancer (e.g., breast cancer).

Thus, in an aspect is provided a method of treating cancer in a subject in need thereof, the method including administering to a subject a therapeutically effective amount of an antibody as provided herein including embodiments thereof. In embodiments, the cancer is metastatic cancer. In embodiments, the cancer is B cell leukemia, mantle cell lymphoma (MCL), Burkett's Lymphoma, lymphoma, chronic lymphocytic leukemia (CLL), Acute Myeloid Leukemia (AML), B-Cell Acute Lymphoblastic Leukemia (B-ALL), T-cell acute lymphoblastic leukemia (T-ALL), renal cancer, colon cancer, breast cancer, ovarian cancer, lung cancer, skin cancer, pancreatic cancer, testicular cancer, bladder cancer, uterine cancer, prostate cancer, or adrenal cancer. In embodiments, the cancer is B cell leukemia. In embodiments, the cancer is mantle cell lymphoma (MCL). In embodiments, the cancer is Burkett's Lymphoma. In embodiments, the cancer is lymphoma. In embodiments, the cancer is chronic lymphocytic leukemia (CLL). In embodiments, the cancer is Acute Myeloid Leukemia (AML). In embodiments, the cancer is B-Cell Acute Lymphoblastic Leukemia (B-ALL). In embodiments, the cancer is T-cell acute lymphoblastic leukemia (T-ALL). In embodiments, the cancer is renal cancer. In embodiments, the cancer is colon cancer. In embodiments, the cancer is breast cancer. In embodiments, the cancer is ovarian cancer. In embodiments, the cancer is lung cancer. In embodiments, the cancer is skin cancer. In embodiments, the cancer is pancreatic cancer. In embodiments, the cancer is testicular cancer. In embodiments, the cancer is bladder cancer. In embodiments, the cancer is uterine cancer. In embodiments, the cancer is prostate cancer. In embodiments, the cancer is adrenal cancer.

In embodiments, the cancer is a solid tumor malignancy. In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

In another aspect is provided a method of inhibiting metastasis of a ROR1 expressing cancer in a subject in need thereof, the method including administering to a subject a therapeutically effective amount of an antibody provided herein including embodiments thereof.

In an aspect is provided a method of detecting a ROR1 expressing cell, the method including (i) contacting a ROR1-expressing cell with an antibody provided herein including embodiments thereof; (ii) and detecting binding of the antibody to a ROR1 protein expressed by the cell. In embodiments, the antibody is attached to a detectable moiety.

In an aspect is a method of delivering a therapeutic agent to a ROR1 expressing cell, the method including contacting a ROR1 expressing cell with an antibody provided herein including embodiments thereof, wherein the antibody is attached to a therapeutic agent. In embodiments, the therapeutic agent is an anti-cancer agent. Exemplary anti-cancer agents include without limitation any anti-cancer agent conventionally used and known in the art, for example, calicheamicin, duocarmycin, pyrrolobenzodiazepine, (PBD), SN-38, DXd and anti-tubulin. Methods for generating antibody drug conjugates are well known in the art and described for example, by Hafeez, U. et al. Antibody-Drug Conjugates for Cancer Therapy; Molecules 2020, 25, 4764; doi:10.3390/molecules25204764.; Ponziani, S. et al. Antibody-Drug Conjugates; The New Frontier of Chemotherpy. Int. J. Mol. Sci. 2020, 21, 5510; doi:10.3390/ijms21155510. and; Joubert, N. et al. Antibody-Drug Conjugates: The Last Decade. Pharmaceuticals 2020, 13, 245; doi:10.3390/phi3090245.; which are incorporated by reference herein in their entirety and for all purposes.

For the methods provided herein, in embodiments, the contacting occurs in vitro. In embodiments, the ROR1-expressing cell is in a subject. In embodiments, the subject is a healthy subject. In embodiments, the subject is a subject having cancer. In embodiments, the cancer is a solid tumor malignancy. In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

For the methods provided herein, in embodiments, the ROR1 expressing cell is a cancer cell. In embodiments, the cancer cell is a breast cancer cell, ovarian cancer cell, pancreatic cancer cell, cervical cancer cell, gastric cancer cell, renal cancer cell, head and neck cancer cell, bone cancer cell, skin cancer cell or prostate cancer cell. In embodiments, the cancer cell is a brest cancer cell. In embodiments, the cancer cell is an ovarian cancer cell. In embodiments, the cancer cell is a pancreatic cancer cell. In embodiments, the cancer cell is a cervical cancer cell. In embodiments, the cancer cell is a gastric cancer cell. In embodiments, the cancer cell is a renal cancer cell. In embodiments, the cancer cell is a head and neck cancer cell. In embodiments, the cancer cell is a bone cancer cell. In embodiments, the cancer cell is a skin cancer cell. In embodiments, the cancer cell is a prostate cancer cell.

For the methods provided herein, in embodiments, the antibody is administered at an amount from about 0.01 nM to about 10 nM. In embodiments, the antibody is administered at an amount from about 0.05 nM to about 10 nM. In embodiments, the antibody is administered at an amount from about 0.1 nM to about 10 nM. In embodiments, the antibody is administered at an amount from about 0.5 nM to about 10 nM. In embodiments, the antibody is administered at an amount from about 1 nM to about 10 nM. In embodiments, the antibody is administered at an amount from about 2 nM to about 10 nM. In embodiments, the antibody is administered at an amount from about 4 nM to about 10 nM. In embodiments, the antibody is administered at an amount from about 6 nM to about 10 nM. In embodiments, the antibody is administered at an amount from about 4 nM to about 10 nM. In embodiments, the antibody is administered at an amount from about 8 nM to about 10 nM. In embodiments, the antibody is administered at an amount of about 0.01 nM, 0.05 nM, 0.1 nM, 0.5 nM, 1 nM, 2 nM, 2 nM, 4 nM, 5 nM, 6 nM, 7 nM, 8 nM, 9 nM or 10 nM.

In embodiments, the antibody is administered at an amount from 0.01 nM to 10 nM. In embodiments, the antibody is administered at an amount from 0.05 nM to 10 nM. In embodiments, the antibody is administered at an amount from 0.1 nM to 10 nM. In embodiments, the antibody is administered at an amount from 0.5 nM to 10 nM. In embodiments, the antibody is administered at an amount from 1 nM to 10 nM. In embodiments, the antibody is administered at an amount from 2 nM to 10 nM. In embodiments, the antibody is administered at an amount from 4 nM to 10 nM. In embodiments, the antibody is administered at an amount from 6 nM to 10 nM. In embodiments, the antibody is administered at an amount from 4 nM to 10 nM. In embodiments, the antibody is administered at an amount from 8 nM to 10 nM. In embodiments, the antibody is administered at an amount of 0.01 nM, 0.05 nM, 0.1 nM, 0.5 nM, 1 nM, 2 nM, 2 nM, 4 nM, 5 nM, 6 nM, 7 nM, 8 nM, 9 nM or 10 nM.

In embodiments, the antibody is administered at an amount from about 0.01 nM to about 8 nM. In embodiments, the antibody is administered at an amount from about 0.05 nM to about 8 nM. In embodiments, the antibody is administered at an amount from about 0.1 nM to about 8 nM. In embodiments, the antibody is administered at an amount from about 0.5 nM to about 8 nM. In embodiments, the antibody is administered at an amount from about 1 nM to about 8 nM. In embodiments, the antibody is administered at an amount from about 2 nM to about 8 nM. In embodiments, the antibody is administered at an amount from about 4 nM to about 8 nM. In embodiments, the antibody is administered at an amount from about 6 nM to about 8 nM. In embodiments, the antibody is administered at an amount from about 4 nM to about 8 nM.

In embodiments, the antibody is administered at an amount from 0.01 nM to 8 nM. In embodiments, the antibody is administered at an amount from 0.05 nM to 8 nM. In embodiments, the antibody is administered at an amount from 0.1 nM to 8 nM. In embodiments, the antibody is administered at an amount from 0.5 nM to 8n M. In embodiments, the antibody is administered at an amount from 1 nM to 8 nM. In embodiments, the antibody is administered at an amount from 2 nM to 8 nM. In embodiments, the antibody is administered at an amount from 4 nM to 8 nM. In embodiments, the antibody is administered at an amount from 6 nM to 8 nM. In embodiments, the antibody is administered at an amount from 4 nM to 8 nM.

In embodiments, the antibody is administered at an amount from about 0.01 nM to about 6 nM. In embodiments, the antibody is administered at an amount from about 0.05 nM to about 6 nM. In embodiments, the antibody is administered at an amount from about 0.1 nM to about 6 nM. In embodiments, the antibody is administered at an amount from about 0.5 nM to about 8 nM. In embodiments, the antibody is administered at an amount from about 1 nM to about 6 nM. In embodiments, the antibody is administered at an amount from about 2 nM to about 6 nM. In embodiments, the antibody is administered at an amount from about 4 nM to about 6 nM.

In embodiments, the antibody is administered at an amount from 0.01 nM to 6 nM. In embodiments, the antibody is administered at an amount from 0.05 nM to 6 nM. In embodiments, the antibody is administered at an amount from 0.1 nM to 6 nM. In embodiments, the antibody is administered at an amount from 0.5 nM to 6 nM. In embodiments, the antibody is administered at an amount from 1 nM to 6 nM. In embodiments, the antibody is administered at an amount from 2 nM to 6 nM. In embodiments, the antibody is administered at an amount from 4 nM to 6 nM.

In embodiments, the antibody is administered at an amount from about 0.01 nM to about 4 nM. In embodiments, the antibody is administered at an amount from about 0.05 nM to about 4 nM. In embodiments, the antibody is administered at an amount from about 0.1 nM to about 4 nM. In embodiments, the antibody is administered at an amount from about 0.5 nM to about 4 nM. In embodiments, the antibody is administered at an amount from about 1 nM to about 4 nM. In embodiments, the antibody is administered at an amount from about 2 nM to about 4 nM.

In embodiments, the antibody is administered at an amount from 0.01 nM to 4 nM. In embodiments, the antibody is administered at an amount from 0.05 nM to 4 nM. In embodiments, the antibody is administered at an amount from 0.1 nM to 4 nM. In embodiments, the antibody is administered at an amount from 0.5 nM to 4 nM. In embodiments, the antibody is administered at an amount from 1 nM to 4 nM. In embodiments, the antibody is administered at an amount from 2 nM to 4 nM.

In embodiments, the antibody is administered at an amount from about 0.01 nM to about 2 nM. In embodiments, the antibody is administered at an amount from about 0.05 nM to about 2 nM. In embodiments, the antibody is administered at an amount from about 0.1 nM to about 2 nM. In embodiments, the antibody is administered at an amount from about 0.5 nM to about 2 nM. In embodiments, the antibody is administered at an amount from about 1 nM to about 2 nM.

In embodiments, the antibody is administered at an amount from 0.01 nM to 2 nM. In embodiments, the antibody is administered at an amount from 0.05 nM to 2 nM. In embodiments, the antibody is administered at an amount from 0.1 nM to 2 nM. In embodiments, the antibody is administered at an amount from 0.5 nM to 2 nM. In embodiments, the antibody is administered at an amount from 1 nM to 2 nM.

In embodiments, the antibody is administered at an amount from about 0.01 nM to about 1 nM. In embodiments, the antibody is administered at an amount from about 0.05 nM to about 1 nM. In embodiments, the antibody is administered at an amount from about 0.1 nM to about 1 nM. In embodiments, the antibody is administered at an amount from about 0.5 nM to about 1 nM.

In embodiments, the antibody is administered at an amount from 0.01 nM to 1 nM. In embodiments, the antibody is administered at an amount from 0.05 nM to 1 nM. In embodiments, the antibody is administered at an amount from 0.1 nM to 1 nM. In embodiments, the antibody is administered at an amount from 0.5 nM to 1 nM.

In embodiments, the antibody is administered at an amount of about 3.15 nM. In embodiments, the antibody is administered at an amount of 3.15 nM. In embodiments, the antibody is administered at an amount of about 1.05 nM. In embodiments, the antibody is administered at an amount of 1.05 nM.

It is understood that the the bispecific antibody or the chimeric antigen receptor provided herein including embodiments thereof may be administered at any of the concentrations described herein for the administration of the antibody (e.g., 0.01 nM-10 nM).

In embodiments, the antibody is administered at an amount from about 10 μg to about 500 μg. In embodiments, the antibody is administered at an amount from about 20 μg to about 500 μg. In embodiments, the antibody is administered at an amount from about 30 μg to about 500 μg. In embodiments, the antibody is administered at an amount from about 40 μg to about 500 μg. In embodiments, the antibody is administered at an amount from about 50 μg to about 500 μg. In embodiments, the antibody is administered at an amount from about 60 μg to about 500 μg. In embodiments, the antibody is administered at an amount from about 70 μg to about 500 μg. In embodiments, the antibody is administered at an amount from about 80 μg to about 500 μg. In embodiments, the antibody is administered at an amount from about 90 μg to about 500 μg. In embodiments, the antibody is administered at an amount from about 100 μg to about 500 μg.

In embodiments, the antibody is administered at an amount from about 110 μg to about 500 μg. In embodiments, the antibody is administered at an amount from about 120 μg to about 500 μg. In embodiments, the antibody is administered at an amount from about 130 μg to about 500 μg. In embodiments, the antibody is administered at an amount from about 140 μg to about 500 μg. In embodiments, the antibody is administered at an amount from about 150 μg to about 500 μg. In embodiments, the antibody is administered at an amount from about 160 μg to about 500 μg. In embodiments, the antibody is administered at an amount from about 170 μg to about 500 μg. In embodiments, the antibody is administered at an amount from about 180 μg to about 500 μg. In embodiments, the antibody is administered at an amount from about 190 μg to about 500 μg. In embodiments, the antibody is administered at an amount from about 200 μg to about 500 μg.

In embodiments, the antibody is administered at an amount from about 210 μg to about 500 μg. In embodiments, the antibody is administered at an amount from about 220 μg to about 500 μg. In embodiments, the antibody is administered at an amount from about 230 μg to about 500 μg. In embodiments, the antibody is administered at an amount from about 240 μg to about 500 μg. In embodiments, the antibody is administered at an amount from about 250 μg to about 500 μg. In embodiments, the antibody is administered at an amount from about 260 μg to about 500 μg. In embodiments, the antibody is administered at an amount from about 270 μg to about 500 μg. In embodiments, the antibody is administered at an amount from about 280 μg to about 500 μg. In embodiments, the antibody is administered at an amount from about 290 μg to about 500 μg. In embodiments, the antibody is administered at an amount from about 300 μg to about 500 μg.

In embodiments, the antibody is administered at an amount from about 310 μg to about 500 μg. In embodiments, the antibody is administered at an amount from about 320 μg to about 500 μg. In embodiments, the antibody is administered at an amount from about 330 μg to about 500 μg. In embodiments, the antibody is administered at an amount from about 340 μg to about 500 μg. In embodiments, the antibody is administered at an amount from about 350 μg to about 500 μg. In embodiments, the antibody is administered at an amount from about 360 μg to about 500 μg. In embodiments, the antibody is administered at an amount from about 370 μg to about 500 μg. In embodiments, the antibody is administered at an amount from about 380 μg to about 500 μg. In embodiments, the antibody is administered at an amount from about 390 μg to about 500 μg. In embodiments, the antibody is administered at an amount from about 400 μg to about 500 μg.

In embodiments, the antibody is administered at an amount from about 410 μg to about 500 μg. In embodiments, the antibody is administered at an amount from about 420 μg to about 500 μg. In embodiments, the antibody is administered at an amount from about 430 μg to about 500 μg. In embodiments, the antibody is administered at an amount from about 440 μg to about 500 μg. In embodiments, the antibody is administered at an amount from about 450 μg to about 500 μg. In embodiments, the antibody is administered at an amount from about 460 μg to about 500 μg. In embodiments, the antibody is administered at an amount from about 470 μg to about 500 μg. In embodiments, the antibody is administered at an amount from about 480 μg to about 500 μg. In embodiments, the antibody is administered at an amount from about 490 μg to about 500 μg.

In embodiments, the antibody is administered at an amount from about 10 μg to about 400 μg. In embodiments, the antibody is administered at an amount from about 20 μg to about 400 μg. In embodiments, the antibody is administered at an amount from about 30 μg to about 400 μg. In embodiments, the antibody is administered at an amount from about 40 μg to about 400 μg. In embodiments, the antibody is administered at an amount from about 50 μg to about 400 μg. In embodiments, the antibody is administered at an amount from about 60 μg to about 400 μg. In embodiments, the antibody is administered at an amount from about 70 μg to about 400 μg. In embodiments, the antibody is administered at an amount from about 80 μg to about 400 μg. In embodiments, the antibody is administered at an amount from about 90 μg to about 400 μg. In embodiments, the antibody is administered at an amount from about 100 μg to about 400 μg.

In embodiments, the antibody is administered at an amount from about 10 μg to about 300 μg. In embodiments, the antibody is administered at an amount from about 20 μg to about 300 μg. In embodiments, the antibody is administered at an amount from about 30 μg to about 300 μg. In embodiments, the antibody is administered at an amount from about 40 μg to about 300 μg. In embodiments, the antibody is administered at an amount from about 50 μg to about 300 μg. In embodiments, the antibody is administered at an amount from about 60 μg to about 300 μg. In embodiments, the antibody is administered at an amount from about 70 μg to about 300 μg. In embodiments, the antibody is administered at an amount from about 80 μg to about 300 μg. In embodiments, the antibody is administered at an amount from about 90 μg to about 300 μg. In embodiments, the antibody is administered at an amount from about 100 μg to about 300 μg.

In embodiments, the antibody is administered at an amount from about 10 μg to about 200 μg. In embodiments, the antibody is administered at an amount from about 20 μg to about 200 μg. In embodiments, the antibody is administered at an amount from about 30 μg to about 200 μg. In embodiments, the antibody is administered at an amount from about 40 μg to about 200 μg. In embodiments, the antibody is administered at an amount from about 50 μg to about 200 μg. In embodiments, the antibody is administered at an amount from about 60 μg to about 200 μg. In embodiments, the antibody is administered at an amount from about 70 μg to about 200 μg. In embodiments, the antibody is administered at an amount from about 80 μg to about 200 μg. In embodiments, the antibody is administered at an amount from about 90 μg to about 200 μg. In embodiments, the antibody is administered at an amount from about 100 μg to about 200 μg.

In embodiments, the antibody is administered at an amount from about 10 μg to about 100 μg. In embodiments, the antibody is administered at an amount from about 20 μg to about 100 μg. In embodiments, the antibody is administered at an amount from about 30 μg to about 100 μg. In embodiments, the antibody is administered at an amount from about 40 μg to about 100 μg. In embodiments, the antibody is administered at an amount from about 50 μg to about 100 μg. In embodiments, the antibody is administered at an amount from about 60 μg to about 100 μg. In embodiments, the antibody is administered at an amount from about 70 μg to about 100 μg. In embodiments, the antibody is administered at an amount from about 80 μg to about 100 μg. In embodiments, the antibody is administered at an amount from about 90 μg to about 100 μg.

In embodiments, the antibody is administered at an amount of about 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, or 500 μg.

In embodiments, the antibody is administered at an amount of 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, or 500 μg.

It is understood that the bispecific antibody or the chimeric antigen receptor provided herein including embodiments thereof may be administered at any of the concentrations described herein for the administration of the antibody (e.g., 10 μg-500 μg).

### Methods of Identifying an Antibody

The compositions provided herein, including embodiments thereof, are further contemplated for identifying an antibody. Thus, in an aspect is provided a method of identifying an anti-ROR1 antibody, the method including: (i) contacting an antibody with a first ROR1 polypeptide comprising a threonine at a position corresponding to position 346 of SEQ ID NO:30; (ii) detecting the antibody binding to the first ROR1 polypeptide; (iii) contacting the antibody with a second ROR 1 polypeptide not including a threonine at a position corresponding to position 346 of SEQ ID NO:30; and (iv) detecting the antibody not binding to the second ROR1 polypeptide, thereby identifying an anti-ROR1 antibody.

In embodiments, the second ROR1 polypeptide includes a serine at a position corresponding to position 346 of SEQ ID NO:30. In embodiments, the first ROR1 polypeptide is a first truncated ROR1 polypoptide. In embodiments, the first truncated ROR1 polypeptide includes amino acid residues 311-393 of the sequence of SEQ ID NO:30. In embodiments, the second ROR1 polypeptide is a second truncated ROR1 polypeptide. In embodiments, the second truncated ROR1 polypeptide includes amino acid residues 311-393 of the sequences of SEQ ID NO:31. In embodiments, the antibody is a humanized antibody. In embodiments, the antibody is an antibody fragment. In embodiments, the antibody is a chimeric antibody. In embodiments, the antibody is a single chain antibody.

### Methods of Inhibiting Cell Migration

The compositions provided herein, including embodiments thereof, are further contemplated for inhibiting cell migration. Thus, in an aspect is provided a method of inhibiting migration of a ROR1-expressing cell, the method including contacting a ROR1 expressing cell with an antibody provided herein including embodiments thereof. In embodiments, the antibody includes a heavy chain variable domain and a light chain variable domain, wherein the heavy chain variable domain includes the sequence of SEQ ID NO:2, SEQ ID NO:6, SEQ ID NO:10, SEQ ID NO:14, SEQ ID NO:18, SEQ ID NO:22, or SEQ ID NO:26; and wherein the light chain variable domain includes the sequence of SEQ ID NO:4, SEQ ID NO:8, SEQ ID NO:12, SEQ ID NO:16, SEQ ID NO:20, SEQ ID NO:24, or SEQ ID NO:28.

For the methods provided herein, in embodiments, the ROR1 expressing cell is a cancer cell. In embodiments, the cancer cell is a breast cancer cell, ovarian cancer cell, pancreatic cancer cell, cervical cancer cell, gastric cancer cell, renal cancer cell, head and neck cancer cell, bone cancer cell, skin cancer cell or prostate cancer cell. In embodiments, the cancer is breast cancer. In embodiments, the cancer cell is an ovarian cancer cell. In embodiments, the cancer cell is a pancreatic cancer cell. In embodiments, the cancer cell is a cervical cancer cell. In embodiments, the cancer cell is a gastric cancer cell. In embodiments, the cancer cell is a renal cancer cell. In embodiments, the cancer cell is a head and neck cancer cell. In embodiments, the cancer cell is a bone cancer cell. In embodiments, the cancer cell is a skin cancer cell. In embodiments, the cancer cell is a prostate cancer cell.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

ROR1, receptor tyrosine kinase like orphan receptor 1, is a cell-surface protein, which is expressed at high levels during embryogenesis, when it plays a role in organogenesis and development of the skeleton, lungs, and nervous system. We found that ROR1 is a receptor for the non-canonical Wnt factor, Wnt5a, which also is important for organogenesis. The expression of decreases during fetal development, becoming negligible on most tissues at term.

We found that ROR1 is expressed by the neoplastic cells of a large variety of different cancers. Moreover, we found that ROR1 plays a functional role that contributes to the migration (e.g. metastasis), growth, and survival of cancer cells. Because of its developmentally restricted expression, tumor cells can be distinguished from normal cells by the distinctive expression of ROR1, which can be targeted for the treatment of patients with cancer.

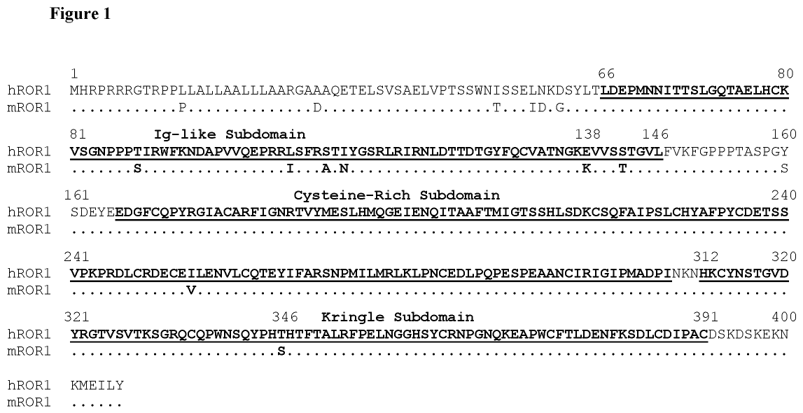

We have generated high-affinity, highly-specific monoclonal antibodies and antibody fragments (henceforth called “mAbs”) that can bind the extracellular domain of human ROR1 (hROR1), but that do not react with the extracellular domain of mouse ROR1 (mROR1), even though the latter shares with hROR1 96% amino acid sequence identity (FIG. 1). These antibodies are designated in this disclosure as UC-NNN and the heavy and light chain sequences as shown in the informal sequence listing below.

For mAb production, mice were inoculated with DNA, protein and adenovirus vectors encoding the extracellular portion (AA 1-406) of hROR1 protein, which is comprised of three major subdomains: the immunoglobulin-like (Ig-like) domain, the cysteine-rich domain (CRD), and the Kringle domain (FIG. 1). To enhance development of high-affinity and specific antibodies to hROR1, we co-injected our vaccines with cytokines and immune-stimulatory factors and allowed sufficient time to elapse so as to select for antibody-producing cells that made antibodies of high affinity.

The sera from the mice were tested for their ability to bind to the extracellular domain of hROR1, using an enzyme-linked immunosorbent assay (ELISA) with plates coated with hROR1 at high and low concentration. We isolated the spleens of animals that made high-titer anti-ROR1 antisera for the purpose of making mAb-producing hybridomas or recombinant cDNA libraries in expression vectors making antibody fragments that could be screened for binding activity to hROR1.

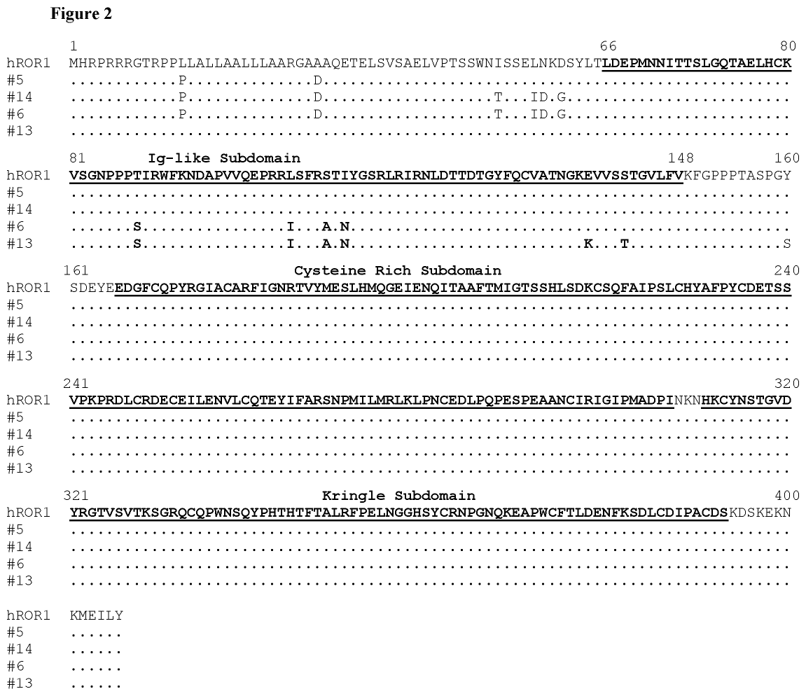

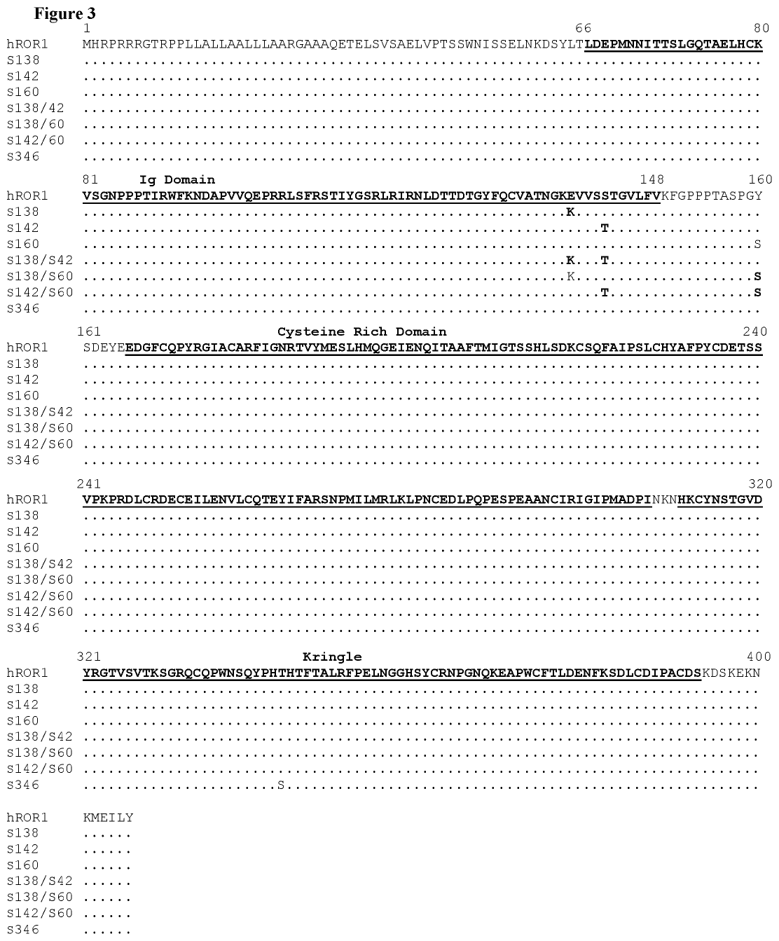

The generated mAbs were screened for their capacity to bind the extracellular domain of hROR1, but not mROR1, via ELISA. We also generated mutant forms of hROR1 that had subdomains deleted. In addition, we generated recombinant forms of hROR1 (#5, #6, #13, #14) that had subdomains of hROR1 or mROR1, allowing us to map the subdomain of hROR1 that was required for antibody binding activity (FIG. 2). Moreover, we used these hybrid ROR1 molecules to map the binding site of each of the anti-hROR1 mAb (FIG. 3).

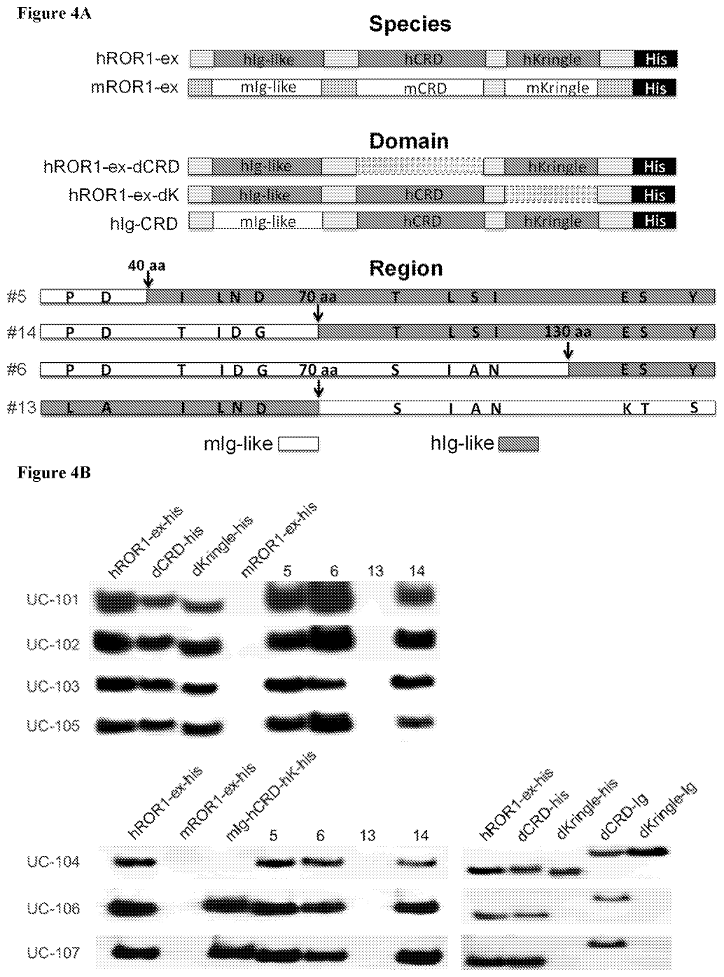

Furthermore, we made single amino acid substitutions in hROR1 at sites that differed from the sequence of mROR1, substituting amino acid residue found in hROR1 with the amino acid residue found in mROR1. This allowed us to map the epitope of hROR1 within the subdomain of hROR1 that was bound by each mAb (FIG. 4).

We found that one set of mAbs were highly specific for an epitope within the Ig-like domain of hROR1, defined by the glutamic acid (E) at position 138 of the hROR1 protein (FIG. 1). These mAbs are each designated as UC-101, UC-102, UC-103, UC-104, or UC-105. The mAb have the heavy chain and light chain sequences provided in SEQ ID NOs:1 through 20. Each of these mAb can bin to chimeric human-mouse ROR1 when the Ig-domain of the chimeric protein was identical to that of hROR1. Moreover, we found these mAb could no longer bind to hROR1 that had a single amino acid substitution at position 138, which exchanged the glutamic acid (E) at this position of hROR1 with the lysine (K) found at this position in the mROR1 protein (FIG. 1).

We found another set of mAbs were highly specific for an epitope within the Kringle domain of hROR1, defined by the threonine (T) at position 346 of the hROR1 protein (FIG. 1). These mAbs are each designated as UC-106 or UC-107. The mAbs have the heavy chain and light chain sequences provided in SEQ ID NOs:21 through 28. Each of these mAb can bind to chimeric human-mouse ROR1 when the Kringle domain of the chimeric protein is identical to that of hROR1. Moreover, we found these mAb could no longer bind to hROR1 that had a single amino acid substitution at position 346, which exchanged the threonine (T) at this position of hROR1 with the serine (S) found at this position in the mROR1 protein (FIG. 1).

Based on these preclinical findings, we assert that these mAbs have a high potential for use in the treatment of patients with cancer, either as a chimeric human-mouse mAb, humanized mAb, bi-specific antibody, antibody-drug conjugate, or binding domain for chimeric antigen receptors intended to direct cytotoxic T cells or NK cells to kill cancer cells that express ROR1.

### P Embodiments

P Embodiment 1. An anti-tyrosine kinase-like orphan receptor 1 (ROR1) antibody comprising a heavy chain variable domain and a light chain variable domain, wherein said heavy chain variable domain comprises the sequence of SEQ ID NO:2, SEQ ID NO: 6, SEQ ID NO: 10, SEQ ID NO:14, SEQ ID NO:18, SEQ ID NO:22, or SEQ ID NO: 26; and wherein said light chain variable domain comprises the sequence of SEQ ID NO:4, SEQ ID NO:8, SEQ ID NO:12, SEQ ID NO:16, SEQ ID NO:20, SEQ ID NO:24, or SEQ ID NO:28.

P Embodiment 2. The anti-tyrosine kinase-like receptor 1 (ROR1) antibody of P embodiment 1, wherein said heavy chain variable domain comprises the sequence of SEQ ID NO:2 and wherein said light chain variable domain comprises the sequence of SEQ ID NO:4.

P Embodiment 3. The anti-tyrosine kinase-like receptor 1 (ROR1) antibody of P embodiment 1, wherein said heavy chain variable domain comprises the sequence of SEQ ID NO:6 and wherein said light chain variable domain comprises the sequence of SEQ ID NO:8.

P Embodiment 4. The anti-tyrosine kinase-like receptor 1 (ROR1) antibody of P embodiment 1, wherein said heavy chain variable domain comprises the sequence of SEQ ID NO:10 and wherein said light chain variable domain comprises the sequence of SEQ ID NO:12.

P Embodiment 5. The anti-tyrosine kinase-like receptor 1 (ROR1) antibody of P embodiment 1, wherein said heavy chain variable domain comprises the sequence of SEQ ID NO:14 and wherein said light chain variable domain comprises the sequence of SEQ ID NO:16.

P Embodiment 6. The anti-tyrosine kinase-like receptor 1 (ROR1) antibody of P embodiment 1, wherein said heavy chain variable domain comprises the sequence of SEQ ID NO:18 and wherein said light chain variable domain comprises the sequence of SEQ ID NO:20.

P Embodiment 7. The anti-tyrosine kinase-like receptor 1 (ROR1) antibody of P embodiment 1, wherein said heavy chain variable domain comprises the sequence of SEQ ID NO:22 and wherein said light chain variable domain comprises the sequence of SEQ ID NO:24.

P Embodiment 8. The anti-tyrosine kinase-like receptor 1 (ROR1) antibody of P embodiment 1, wherein said heavy chain variable domain comprises the sequence of SEQ ID NO:26 and wherein said light chain variable domain comprises the sequence of SEQ ID NO:28.

P Embodiment 9. The anti-tyrosine kinase-like receptor 1 (ROR1) antibody of any one of P embodiments 1-8, wherein said antibody binds to the Kringle domain of human ROR1.

P Embodiment 10. The anti-tyrosine kinase-like receptor 1 (ROR1) antibody of any one of P embodiments 1-8, wherein said antibody binds to the immunoglobulin-like (Ig-like) domain of human ROR1.

P Embodiment 11. A method of preventing or treating cancer in a subject in need thereof, said method comprising administering to a subject a therapeutically effective of an antibody of any one of P embodiments 1-10.

P Embodiment 12. A pharmaceutical composition comprising an antibody of any one of P embodiments 1-11 and a pharmaceutically effective excipient.

### EMBODIMENTS

Embodiment 1. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:36, a CDR H2 as set forth in SEQ ID NO:38, and a CDR H3 as set forth in SEQ ID NO:40; and wherein said light chain variable domain comprises: a CDR L1 as set forth in SEQ ID NO:43, a CDR L2 as set forth in SEQ ID NO:45 and a CDR L3 as set forth in SEQ ID NO:47.

Embodiment 2. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:64, a CDR H2 as set forth in SEQ ID NO:66, and a CDR H3 as set forth in SEQ ID NO:68; and wherein said light chain variable domain comprises: a CDR L1 as set forth in SEQ ID NO:71, a CDR L2 as set forth in SEQ ID NO: 73 and a CDR L3 as set forth in SEQ ID NO:75.

Embodiment 3. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:78, a CDR H2 as set forth in SEQ ID NO:80, and a CDR H3 as set forth in SEQ ID NO:82; and wherein said light chain variable domain comprises: a CDR L1 as set forth in SEQ ID NO:85, a CDR L2 as set forth in SEQ ID NO: 87 and a CDR L3 as set forth in SEQ ID NO: 89.

Embodiment 4. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:92, a CDR H2 as set forth in SEQ ID NO:94, and a CDR H3 as set forth in SEQ ID NO:96; and wherein said light chain variable domain comprises: a CDR L1 as set forth in SEQ ID NO:99, a CDR L2 as set forth in SEQ ID NO:101 and a CDR L3 as set forth in SEQ ID NO: 103.

Embodiment 5. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:106, a CDR H2 as set forth in SEQ ID NO:108, and a CDR H3 as set forth in SEQ ID NO:110; and wherein said light chain variable domain comprises: a CDR L1 as set forth in SEQ ID NO: 113, a CDR L2 as set forth in SEQ ID NO:115 and a CDR L3 as set forth in SEQ ID NO: 117.

Embodiment 6. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:50, a CDR H2 as set forth in SEQ ID NO:52, and a CDR H3 as set forth in SEQ ID NO:54; and wherein said light chain variable domain comprises: a CDR L1 as set forth in SEQ ID NO:57, a CDR L2 as set forth in SEQ ID NO:59 and a CDR L3 as set forth in SEQ ID NO:61.

Embodiment 7. An anti-tyrosine kinase-like orphan receptor 1 (ROR1) antibody comprising a heavy chain variable domain and a light chain variable domain, wherein said heavy chain variable domain comprises a CDR H1 as set forth in SEQ ID NO:120, a CDR H2 as set forth in SEQ ID NO:122, and a CDR H3 as set forth in SEQ ID NO:124; and wherein said light chain variable domain comprises: a CDR L1 as set forth in SEQ ID NO:127, a CDR L2 as set forth in SEQ ID NO:129 and a CDR L3 as set forth in SEQ ID NO:131.

Embodiment 8. The anti-ROR1 antibody of any one of embodiments 1-7, wherein said antibody is a humanized antibody, a chimeric antibody, a Fab′ fragment or a scFv.

Embodiment 9. The anti-ROR1 antibody of any one of embodiments 1-8, wherein said antibody is a humanized antibody.

Embodiment 10. The anti-ROR1 antibody of any one of embodiments 1-9, wherein said heavy chain variable domain comprises the sequence of SEQ ID NO:2, SEQ ID NO: 6, SEQ ID NO:10, SEQ ID NO:14, SEQ ID NO:18, SEQ ID NO:22 or SEQ ID NO:26.

Embodiment 11. The anti-ROR1 antibody of any one of embodiments 1-10, wherein said light chain variable domain comprises the sequence of SEQ ID NO:4, SEQ ID NO:8, SEQ ID NO:12, SEQ ID NO:16, SEQ ID NO:20, SEQ ID NO:24 or SEQ ID NO:28.

Embodiment 12. The anti-ROR1 antibody of any one of embodiments 1-11, wherein said heavy chain variable domain comprises the sequence of SEQ ID NO:2 and said light chain variable domain comprises the sequence of SEQ ID NO:4.

Embodiment 13. The anti-ROR1 antibody of any one of embodiments 1-11, wherein said heavy chain variable domain comprises the sequence of SEQ ID NO:6 and said light chain variable domain comprises the sequence of SEQ ID NO:8.

Embodiment 14. The anti-ROR1 antibody of any one of embodiments 1-11, wherein said heavy chain variable domain comprises the sequence of SEQ ID NO:10 and said light chain variable domain comprises the sequence of SEQ ID NO:12.

Embodiment 15. The anti-ROR1 antibody of any one of embodiments 1-11, wherein said heavy chain variable domain comprises the sequence of SEQ ID NO:14 and said light chain variable domain comprises the sequence of SEQ ID NO:16.

Embodiment 16. The anti-ROR1 antibody of any one of embodiments 1-11, wherein said heavy chain variable domain comprises the sequence of SEQ ID NO:18 and said light chain variable domain comprises the sequence of SEQ ID NO:20.

Embodiment 17. The anti-ROR1 antibody of any one of embodiments 1-11, wherein said heavy chain variable domain comprises the sequence of SEQ ID NO:22 and said light chain variable domain comprises the sequence of SEQ ID NO:24.

Embodiment 18. The anti-ROR1 antibody of any one of embodiments 1-11, wherein said heavy chain variable domain comprises the sequence of SEQ ID NO:26 and said light chain variable domain comprises the sequence of SEQ ID NO:28.

Embodiment 19. The anti-ROR1 antibody of any one of embodiments 1-5, 10-11 or 12-16, wherein said anti-ROR1 antibody binds the Ig-like domain of human ROR1.

Embodiment 20. The anti-ROR1 antibody of any one of embodiments 6-7, 10-11 or 17-18, wherein said anti-ROR1 antibody binds the Kringle domain of human ROR1.

Embodiment 21. The anti-ROR1 antibody of any one of embodiments 4-5 or 15-16, wherein said anti-ROR1 antibody binds a ROR1 polypeptide comprising a glutamic acid at a position corresponding to position 138 of SEQ ID NO:30.

Embodiment 22. The anti-ROR1 antibody of embodiments 6 or 17, wherein said anti-ROR1 antibody binds a ROR1 polypeptide comprising a threonine at a position corresponding to position 346 of SEQ ID NO:30.

Embodiment 23. An anti-ROR1 antibody, wherein said anti-ROR1 antibody binds the same epitope as an antibody comprising: a heavy chain variable domain comprising a CDR H1 as set forth in SEQ ID NO:36, a CDR H2 as set forth in SEQ ID NO:38 and a CDR H3 as set forth in SEQ ID NO:40; and a light chain variable domain comprising a CDR L1 as set forth in SEQ ID NO:43, a CDR L2 as set forth in SEQ ID NO:45, and a CDR L3 as set forth in SEQ ID NO:47.

Embodiment 24. An anti-ROR1 antibody, wherein said anti-ROR1 antibody binds the same epitope as an antibody comprising: a heavy chain variable domain comprising a CDR H1 as set forth in SEQ ID NO:64, a CDR H2 as set forth in SEQ ID NO:66 and a CDR H3 as set forth in SEQ ID NO:68; and a light chain variable domain comprising a CDR L1 as set forth in SEQ ID NO:71, a CDR L2 as set forth in SEQ ID NO:73, and a CDR L3 as set forth in SEQ ID NO: 75.

Embodiment 25. An anti-ROR1 antibody, wherein said anti-ROR1 antibody binds the same epitope as an antibody comprising: a heavy chain variable domain comprising a CDR H1 as set forth in SEQ ID NO:78, a CDR H2 as set forth in SEQ ID NO:80 and a CDR H3 as set forth in SEQ ID NO:82; and a light chain variable domain comprising a CDR L1 as set forth in SEQ ID NO:85, a CDR L2 as set forth in SEQ ID NO:87, and a CDR L3 as set forth in SEQ ID NO: 89.

Embodiment 26. An anti-ROR1 antibody, wherein said anti-ROR1 antibody binds the same epitope as an antibody comprising: a heavy chain variable domain comprising a CDR H1 as set forth in SEQ ID NO:92, a CDR H2 as set forth in SEQ ID NO:94 and a CDR H3 as set forth in SEQ ID NO:96; and a light chain variable domain comprising a CDR L1 as set forth in SEQ ID NO:99, a CDR L2 as set forth in SEQ ID NO:101, and a CDR L3 as set forth in SEQ ID NO: 103.

Embodiment 27. An anti-ROR1 antibody, wherein said anti-ROR1 antibody binds the same epitope as an antibody comprising: a heavy chain variable domain comprising a CDR H1 as set forth in SEQ ID NO:106, a CDR H2 as set forth in SEQ ID NO:108 and a CDR H3 as set forth in SEQ ID NO:110; and a light chain variable domain comprising a CDR L1 as set forth in SEQ ID NO:113, a CDR L2 as set forth in SEQ ID NO:115, and a CDR L3 as set forth in SEQ ID NO: 117.

Embodiment 28. An anti-ROR1 antibody, wherein said anti-ROR1 antibody binds the same epitope as an antibody comprising: a heavy chain variable domain comprising a CDR H1 as set forth in SEQ ID NO:50, a CDR H2 as set forth in SEQ ID NO:52 and a CDR H3 as set forth in SEQ ID NO:54; and a light chain variable domain comprising a CDR L1 as set forth in SEQ ID NO:57, a CDR L2 as set forth in SEQ ID NO:59, and a CDR L3 as set forth in SEQ ID NO:61.

Embodiment 29. An anti-ROR1 antibody, wherein said anti-ROR1 antibody binds the same epitope as an antibody comprising: a heavy chain variable domain comprising a CDR H1 as set forth in SEQ ID NO:120, a CDR H2 as set forth in SEQ ID NO:122 and a CDR H3 as set forth in SEQ ID NO:124; and a light chain variable domain comprising a CDR L1 as set forth in SEQ ID NO: 127, a CDR L2 as set forth in SEQ ID NO:129, and a CDR L3 as set forth in SEQ ID NO:131.

Embodiment 30. The anti-ROR1 antibody of any one of embodiments 1-29, wherein said anti-ROR1 antibody is bound to a ROR1 protein.

Embodiment 31. The anti-ROR1 antibody of embodiment 30, wherein said ROR1 protein forms part of a cell.

Embodiment 32. The anti-ROR1 antibody of embodiment 30 or 31, wherein said ROR1 protein is expressed on the surface of a cell.

Embodiment 33. The anti-ROR1 antibody of embodiment 31 or 32, wherein said cell is a cancer cell.

Embodiment 34. The anti-ROR1 antibody of embodiment 33, wherein said cancer cell is a B cell leukemia cell, a mantle cell lymphoma (MCL) cell, a Burkett's Lymphoma cell, a lymphoma cell, a chronic lymphocytic leukemia (CLL) cell, an Acute Myeloid Leukemia (AML) cell, a B-Cell Acute Lymphoblastic Leukemia (B-ALL) cell, a T-cell acute lymphoblastic leukemia (T-ALL) cell, a renal cancer cell, a colon cancer cell, a breast cancer cell, an ovarian cancer cell, a lung cancer cell, a skin cancer cell, a pancreatic cancer cell, a testicular cancer cell, a bladder cancer cell, a uterine cancer cell, a prostate cancer cell, or an adrenal cancer cell.

Embodiment 35. The anti-ROR1 antibody of any one of embodiments 1-34, wherein said anti-ROR1 antibody is bound to a therapeutic moiety or a diagnostic moiety.

Embodiment 36. A cell comprising an anti-ROR1 antibody of any one of embodiments 1-35.

Embodiment 37. A nucleic acid encoding an anti-ROR1 antibody of any one of embodiments 1-35.

Embodiment 38. A pharmaceutical composition comprising a therapeutically effective amount of an anti-ROR1 antibody of any one of embodiments 1-35, and a pharmaceutically acceptable excipient.

Embodiment 39. A method of treating cancer in a subject in need thereof, said method comprising administering to a subject a therapeutically effective amount of an anti-ROR1 antibody of any one of embodiments 1-35.

Embodiment 40. The method of embodiment 39, wherein said cancer is B cell leukemia, mantle cell lymphoma (MCL), Burkett's Lymphoma, lymphoma, chronic lymphocytic leukemia (CLL), Acute Myeloid Leukemia (AML), B-Cell Acute Lymphoblastic Leukemia (B-ALL), T-cell acute lymphoblastic leukemia (T-ALL), renal cancer, colon cancer, breast cancer, ovarian cancer, lung cancer, skin cancer, pancreatic cancer, testicular cancer, bladder cancer, uterine cancer, prostate cancer, or adrenal cancer.

Embodiment 41. A method of identifying an anti-ROR1 antibody, the method comprising: (i) contacting an antibody with a first ROR1 polypeptide comprising a threonine at a position corresponding to position 346 of SEQ ID NO:30; (ii) detecting said antibody binding to said first ROR1 polypeptide; (iii) contacting said antibody with a second ROR 1 polypeptide not comprising a threonine at a position corresponding to position 346 of SEQ ID NO:30; and (iv) detecting said antibody not binding to said second ROR1 polypeptide, thereby identifying an anti-ROR1 antibody.

Embodiment 42. The method of embodiment 41, wherein said second ROR1 polypeptide comprises a serine at a position corresponding to position 346 of SEQ ID NO:30.

Embodiment 43. The method of embodiment 41 or 42, wherein said first ROR1 polypeptide is a first truncated ROR1 polypeptide.

Embodiment 44. The method of embodiment 43, wherein said first truncated ROR1 polypeptide comprises amino acid residues 311-393 of the sequence of SEQ ID NO:30.

Embodiment 45. The method of embodiment 43 or 44, wherein said second ROR1 polypeptide is a second truncated ROR1 polypeptide.

Embodiment 46. The method of embodiment 45, wherein said second truncated ROR1 polypeptide comprises amino acid residues 311-393 of the sequence of SEQ ID NO:31.

Embodiment 47. The method of any one of embodiments 41-46, wherein said antibody is a humanized antibody.

Embodiment 48. The method of any one of embodiments 41-46, wherein said antibody is an antibody fragment.

Embodiment 49. The method of any one of embodiments 41-46, wherein said antibody is a chimeric antibody.

Embodiment 50. The method of any one of embodiments 41-46, wherein said antibody is a single chain antibody.

INFORMAL SEQUENCE LISTING

SEQ ID NO: 1; DNA; UC-101 VH SEQ
GAAGTGCAGCTGTTGGAGACTGGGGGAGGCTTAGTGCAGCCTGGAGGGTCCCGG
AAACTCTCCTGTGTAGCCTCTGGATTCAGTTTCAGTAGCTATGGAATGCACTGGG
TTCGTCAGGCTCCAGAGAAGGGGCTGGAGTGGGTCGCATATATTGGTGGTACCA
GTAATACCATCTACTTTGCAGACACAGTGAAGGGCCGATTCACCATTTCCAGAGA
CAATGCCAAGAACACCCTGTTCCTGCAAATGGCCAGTCTAAGGTCTGAGGACAC
GGCCATGTATTACTGTGCAAGAACGAACTTACTCGGGATGGACTACTGGGGTCA
AGGAACCTCAGTCACCGTCTCCTCA
SEQ ID NO: 2; PRT; UC-101 VH SEQ
EVQLLETGGGLVQPGGSRKLSCVASGFSFSSYGMHWVRQAPEKGLEWVAYIGGTSN
TIYFADTVKGRFTISRDNAKNTLFLQMASLRSEDTAMYYCARTNLLGMDYWGQGTS
VTVSS
SEQ ID NO: 3; DNA; UC-101 VK SEQ
CAAATTGTTCTCACCCAGTCTCCAACAATCATGTCTGCATCTCCAGGGGAGAAGG
TCACCATGACCTGCAGTGCCAGCTCAAGTGTAAGTGACATGTACTGGTACCAGCA
GAAGCCAGGATCCTCCCCCAGACTCCTCATTTATGACACATCCAACCTGGCTTCT
GGAGTCCCTGTTCGCTTCAGTGGCAGTGGGTCTGGGACCTCTTACTCTCTCACAA
TCAGCCGAATGGAGGCTGAAGATGCTGCCACTTATTACTGCCAGCAGTGGATTA
GTTACACGTTCGGAGGGGGGACCAAGCTGGAAATAAAACGG
SEQ ID NO: 4; PRT; UC-101 VK SEQ
QIVLTQSPTIMSASPGEKVTMTCSASSSVSDMYWYQQKPGSSPRLLIYDTSNLASGVP
VRFSGSGSGTSYSLTISRMEAEDAATYYCQQWISYTFGGGTKLEIKR
SEQ ID NO: 5; DNA; UC-102 VH SEQ
GATGTGCACCTTCAGGAGTCTGGACCTGGCCTGGTGAAACCTTCTCAGTCTCTAT
CTGTCACCTGCACTGTCACTGGCTACTCCATCACCGGTAGTTATAGCTGGAACTG
GATCCGGCAGTTTCCAGGAAACAAACTGGAGTGGATGGGCTACATACACTCCAG
TGGTACCACTAACTACAACCCATCTCTCAAAAGTCGAATCTCTTTTACTCGAGAC
ACATCCAAGAACCAACTCTTCCTGCAGTTGAATTCTGTGACTACTGAGGACACAG
CCACATATTACTGTACAAGGGGGTTTGCTTACTGGGGCCAAGGGACTCTGGTCAC
TGTCTCTGCA
SEQ ID NO: 6; PRT; UC-102 VH SEQ
DVHLQESGPGLVKPSQSLSVTCTVTGYSITGSYSWNWIRQFPGNKLEWMGYIHSSGT
TNYNPSLKSRISFTRDTSKNQLFLQLNSVTTEDTATYYCTRGFAYWGQGTLVTVSA
SEQ ID NO: 7; DNA; UC-102 VK SEQ
AACATCGTTATGACCCAGTCTCCATCCTCTATGTCTGCATCTCTGGGAGACAGAA
TAACCATCACTTGCCAGGCAACTCAAGACATTGTTAAGAATTTAAACTGGTATCA
GCAGAAACCAGGGAAACCCCCTTCATTCCTGATCTATTATACAACTGAACTGGCA
GAAGGGGTCCCATCAAGGTTCAGTGGCAGTGGGTCTGGGTCAGACTATTCTCTGA
CAATCAGCAACCTGGAGTCTGAAGATTTTGCAGACTATTACTGTCTTCAGTTTTAT
GAGTTTCCTCCCACGTTCGGTGCTGGGACCAAGCTGGAGCTGAAACGG
SEQ ID NO: 8; PRT; UC-102 VK SEQ
NIVMTQSPSSMSASLGDRITITCQATQDIVKNLNWYQQKPGKPPSFLIYYTTELAEGV
PSRFSGSGSGSDYSLTISNLESEDFADYYCLQFYEFPPTFGAGTKLELKR
SEQ ID NO: 9; DNA; UC-103 VH SEQ
GAGATCCAGCTGCAGCAGTCTGGACCTGTCCTGGTGAAGCCTGGGGCTTCAGTG
AAGGTTTCTTGCAAGGCTTCTGGTTATGCATTCACTGCCTACAACATACACTGGG
TGAGACAGAGCCATGGAAAGCGCCTTGAGTGGATTGGATCTTTTGATCCTTACGA
TGGTGGTAGTAGTTACAACCAGAAGTTCAAGGACAAAGCCACATTGACTGTAGA
CAAATCTTCCACCACAGCCTACATGCATCTCAACAGCCTGACATCTGAGGACTCT
GCAGTCTATTACTGTGCAAGGGGGTGGTACTACTTTGACTACTGGGGCCACGGGA
CCACTCTCACAGTCTCCTCA
SEQ ID NO: 10; PRT; UC-103 VH SEQ
EIQLQQSGPVLVKPGASVKVSCKASGY AFTAYNIHWVRQSHGKRLEWIGSFDPYDG
GSSYNQKFKDKATLTVDKSSTTAYMHLNSLTSEDSAVYYCARGWYYFDYWGHGTT
LTVSS
SEQ ID NO: 11; DNA; UC-103 VK SEQ
GACGTCCAGATAACCCAGTCTCCATCTTATCTTGCTGCATCTCCTGGAGAAACCA
TTACTATTAATTGCAGGGCAAGTAAGAGCATTAGCAAATATTTAGCCTGGTATCA
AGAGAAACCTGGGAAAACTAATAAGCTCCTTATCTACTCTGGATCCACTTTGCAA
TCTGGAATTCCATCAAGATTCAGGGGCAGTGGATCTGGTACAGATTTCACTCTCA
CCATCAGTAGCCTGGAGCCTGAAGATTTTGCAATGTATTACTGTCAACAGCATGA
TGAATCCCCGTACACGTTCGGAGAGGGGACCAAGCTGGAAATAAAACGG
SEQ ID NO: 12; PRT; UC-103 VK SEQ
DVQITQSPSYLAASPGETITINCRASKSISKYLAWYQEKPGKTNKLLIYSGSTLQSGIPS
RFRGSGSGTDFTLTISSLEPEDFAMYYCQQHDESPYTFGEGTKLEIKR
SEQ ID NO: 13; DNA; UC-104 VH SEQ
GAAGTGCAGCTGTTGGAGACTGGGGGAGGCTTAGTGCAGCCTGGAGGGTCCCGG
AAACTCTCCTGTGCAGCCTCTGGATTCACTTTCAGTAACTATGGAATGCACTGGG
TTCGTCAGGCTCCAGGGAAGGGGCTGGAGTGGGTCGCACATATTAGTCGTTACA
GTGATACCATCTACTATGCAGACGCAGTGAAGGGCCGATTCACCATCTCCAGAG
ACAATGCCAAGAACACCCTGTTCCTGCAAATGACCACTCTAAGGTCTGAGGACA
CGGCCATATATTACTGTACAAGTGCTATGGACTACTGGGGTCAAGGAACCTCAGT
CACCGTCTCCTCA
SEQ ID NO: 14; PRT; UC-104 VH SEQ
EVQLLETGGGLVQPGGSRKLSCAASGFTFSNYGMHWVRQAPGKGLEWVAHISRYS
DTIYYADAVKGRFTISRDNAKNTLFLQMTTLRSEDTAIYYCTSAMDYWGQGTSVTV
SS
SEQ ID NO: 15; DNA; UC-104 VK SEQ
GATATCCAGATGACACAGACTACATCCTCCCTGTCTGCCTCTCTGGGAGACAGAG
TCACCATCAGTTGCAGGGCAAGTCAGGACATTAGCAATTATTTAAACTGGTTTCA
GCAGAAACCAGATGGAACTATTAAACTCCTGATCTACTACACATCAAGATTACA
CTCAGGAGTCCCATCAAGGTTCAGTGGCAGTGGGTCTGGAACAGATTATTCTCTC
ACCATTAGCAACCTGGAACAAGAAGATATTGCCACTTACTTTTGCCAGCAGGGTA
ATACGCTTCCATTCACGTTCGGCTCGGGGACAAAGTTGGAAATAAAACGG
SEQ ID NO: 16; PRT; UC-104 VK SEQ
DIQMTQTTSSLSASLGDRVTISCRASQDISNYLNWFQQKPDGTIKLLIYYTSRLHSGVP
SRFSGSGSGTDYSLTISNLEQEDIATYFCQQGNTLPFTFGSGTKLEIKR
SEQ ID NO: 17; DNA; UC-105 VH SEQ
GAGGTGAAGCTGGTGGAATCTGGGGGAGGCTTAGTGCAGCCTGGAGGGTCCCGG
AAACTCTCCTGTGCAGCCTCTGGATTCACTTTCAGTAGCTATGGAATGCACTGGA
TTCGTCAGACTCCAGACAAGGGGCTGGAATGGGTCGCATATATTAGTAGTAACA
GTGATCACATCTTCTATACAGACACAGTGAAGGGCCGATTCACCATCTCCAGAGA
CAATGCCAAGAACACCCTGTTCCTGCAAATGACCAGTCTAAGGTCTGAGGACAC
GGCCATGTATTACTGTGCAAGCCGTATGGACTACTGGGGTCAAGGAACCTCAGTC
ACCGTCTCCTCA
SEQ ID NO: 18; PRT; UC-105 VH SEQ
EVKLVESGGGLVQPGGSRKLSCAASGFTFSSYGMHWIRQTPDKGLEWVAYISSNSD
HIFYTDTVKGRFTISRDNAKNTLFLQMTSLRSEDTAMYYCASRMDYWGQGTSVTVS
S
SEQ ID NO: 19; DNA; UC-105 VK SEQ
GATATCCAGATGACACAGACTACATCCTCCCTGTCTGCCTCTCTGGGAGACAGAG
TCACCATCAGTTGCAGGGCAAGTCAGGACATTAGCAATCATTTAAACTGGTATCA
GCAGAAACCAGATGGAACTGTTAAACTCCTGATCTACTACACATCAAGATTACA
CTCAGGAGTCCCATCAAGGTTCAGTGGCAGTGGGTCTGGAACAGATTATTCTCTC
ACCATTAGCAACCTGGAGCAAGGCGATATTGCCACTTACTTTTGCCAACAGGGTA
GTACGCTTCCGTACACATTCGGAGGGGGGACCAAGCTGGAAATAAAACGG
SEQ ID NO: 20; PRT; UC-105 VK SEQ
DIQMTQTTSSLSASLGDRVTISCRASQDISNHLNWYQQKPDGTVKLLIYYTSRLHSGV
PSRFSGSGSGTDYSLTISNLEQGDIATYFCQQGSTLPYTFGGGTKLEIKR
SEQ ID NO: 21; DNA; UC-106 VH SEQ
CAGATCCAGTTGGTGCAATCTGGACCTGAGCTGAAGAAGCCTGGAGAGACAGTC
AAGATCTCCTGCAAGGCTTCTGGGTATACCTTCACATACTATCCAATCCACTGGG
TGAAGCAGGCTCCAGGAAAGGGTTTAAAGTGGATGGGCTGGATAAACACCTACT
CTGGAGTGCCAACATATGCAGATGACTTCAAGGGACGGTTTGCCTTCTCTTTGGA
AACCTCTGCCAACACTGCATATTTGCAGATCAACAACCTCAGAAATGAAGACAT
GGCTACATATTTCTGTGCAAGGGGGGATAGTAGCCTTTTTGACTACTGGGGCCAA
GGCACCACTCTCACAGTCTCCTCA
SEQ ID NO: 22; PRT; UC-106 VH SEQ
QIQLVQSGPELKKPGETVKISCKASGYTFTYYPIHWVKQAPGKGLKWMGWINTYSG
VPTYADDFKGRFAFSLETSANTAYLQINNLRNEDMATYFCARGDSSLFDYWGQGTT
LTVSS
SEQ ID NO: 23; DNA; UC-106 VK SEQ
GGACATCAAATGACCCAGTCTCCATCCTCCTTATCTGCCTCTCTGGGAGAAAGAG
TCAGTCTCACTTGTCGGGCAAGTCAGGAAATTAGTGGTTACTTAATCTGGCTTCA
GCAGAAACCGGATGGAACTATTAAACGCCTGATCTACGCCGCATCCACTTTAAAT
TCTGGTGTCCCAAAAAGGTTCAGTGGCAGTAGGTCTGGGTCAGATTACTCTCTCA
CCATCAGCAGCCTTGAGTCTGAAGATTTTGCTGACTATTACTGTCTACAATATAC
TAGTTATCCTCTCACGTTCGGTGCTGGGACCAAGCTGGAGCTGAAACGG
SEQ ID NO: 24; PRT; UC-106 VK SEQ
GHQMTQSPSSLSASLGERVSLTCRASQEISGYLIWLQQKPDGTIKRLIYAASTLNSGV
PKRFSGSRSGSDYSLTISSLESEDFADYYCLQYTSYPLTFGAGTKLELKR
SEQ ID NO: 25; DNA; UC-107 VH SEQ
CAGATCCAGTTGGTGCAATCTGGACCTGAGCTGAAGAAGCCTGGAGAGACAGTC
AAGATCTCCTGCAAGGCTTCTGGATATACCTTCACAAACTATCCAATACACTGGG
TGAAGCAGGCTCCAGGAAAGGGTTTAAAGTGGATGGGCTGGATAAACACCTACT
CTGGAGTGCCAACATATGCAGATGACTTCAAGGGACGGTTTGCCTTCTCTTTGGA
AACCTCTGCCAACACTGCATATTTGCAGCTCAACAACCTCAGAAATGAGGACAT
GGCTACATATTTCTGTGCAAGGGGGGATAGTAGCCTTTTTGACTACTGGGGCCAA
GGCACCACTCTCACAGTCTCCTCA
SEQ ID NO: 26; PRT; UC-107 VH SEQ
QIQLVQSGPELKKPGETVKISCKASGYTFTNYPIHWVKQAPGKGLKWMGWINTYSG
VPTYADDFKGRFAFSLETSANTAYLQLNNLRNEDMATYFCARGDSSLFDYWGQGTT
LTVSS
SEQ ID NO: 27; DNA; UC-107 VK SEQ
GACGTTCAGATGATCCAGTCTCCATCCTCCTTATCTGCCTCTCTGGGAGAAAGAG
TCAGTCTCACTTGTCGGGCAAGTCAGGAAATTAGTGGTTACTTAATCTGGCTTCA
GCAGAAACCAGATGGAACTATTACACGCCTGATCTACGCCGCATCCACTTTAGAT
TCTGGTGTCCCAAAAAGGTTCAGTGGCAGTAGGTCTGGGTCAGATTACTCTCTCA
CCATCAGCAGCCTTGAGTCTGAAGATTTTGCTGACTATTACTGTCTACAATATAC
TAGTTATCCTCTCACGTTCGGTGCTGGGACCAAGCTGGAGCTGAAACGG
SEQ ID NO: 28; PRT; UC-107 VK SEQ
DVQMIQSPSSLSASLGERVSLTCRASQEISGYLIWLQQKPDGTITRLIYAASTLDSGVP
KRFSGSRSGSDYSLTISSLESEDFADYYCLQYTSYPLTFGAGTKLELKR
SEQ ID NO: 29; Human ROR1
MHRPRRRGTRPPLLALLAALLLAARGAAAQETELSVSAELVPTSSWNISSELNKDSY
LTLDEPMNNITTSLGQTAELHCKVSGNPPPTIRWFKNDAPVVQEPRRLSFRSTIYGSR
LRIRNLDTTDTGYFQCVATNGKEVVSSTGVLFVKFGPPPTASPGYSDEYEEDGFCQP
YRGIACARFIGNRTVYMESLHMQGEIENQITAAFTMIGTSSHLSDKCSQFAIPSLCHY
AFPYCDETSSVPKPRDLCRDECEILENVLCQTEYIFARSNPMILMRLKLPNCEDLPQPE
SPEAANCIRIGIPMADPINKNHKCYNSTGVDYRGTVSVTKSGRQCQPWNSQYPHTHT
FTALRFPELNGGHSYCRNPGNQKEAPWCFTLDENFKSDLCDIPACDSKDSKEKNKM
EILYILVPSVAIPLAIALLFFFICVCRNNQKSSSAPVQRQPKHVRGQNVEMSMLNAYK
PKSKAKELPLSAVRFMEELGECAFGKIYKGHLYLPGMDHAQLVAIKTLKDYNNPQQ
WTEFQQEASLMAELHHPNIVCLLGAVTQEQPVCMLFEYINQGDLHEFLIMRSPHSDV
GCSSDEDGTVKSSLDHGDFLHIAIQIAAGMEYLSSHFFVHKDLAARNILIGEQLHVKIS
DLGLSREIYSADYYRVQSKSLLPIRWMPPEAIMYGKFSSDSDIWSFGVVLWEIFSFGL
QPYYGFSNQEVIEMVRKRQLLPCSEDCPPRMYSLMTECWNEIPSRRPRFKDIHVRLRS
WEGLSSHTSSTTPSGGNATTQTTSLSASPVSNLSNPRYPNYMFPSQGITPQGQIAGFIG
PPIPQNQRFIPINGYPIPPGYAAFPAAHYQPTGPPRVIQHCPPPKSRSPSSASGSTSTGHV
TSLPSSGSNQEANIPLLPHMSIPNHPGGMGITVFGNKSQKPYKIDSKQASLLGDANIH
GHTESMISAEL
SEQ ID NO: 30; Human ROR1 extracellular domain
MHRPRRRGTRPPLLALLAALLLAARGAAAQETELSVSAELVPTSSWNISSELNKDSY
LTLDEPMNNITTSLGQTAELHCKVSGNPPPTIRWFKNDAPVVQEPRRLSFRSTIYGSR
LRIRNLDTTDTGYFQCVATNGKEVVSSTGVLFVKFGPPPTASPGYSDEYEEDGFCQP
YRGIACARFIGNRTVYMESLHMQGEIENQITAAFTMIGTSSHLSDKCSQFAIPSLCHY
AFPYCDETSSVPKPRDLCRDECEILENVLCQTEYIFARSNPMILMRLKLPNCEDLPQPE
SPEAANCIRIGIPMADPINKNHKCYNSTGVDYRGTVSVTKSGRQCQPWNSQYPHTHT
FTALRFPELNGGHSYCRNPGNQKEAPWCFTLDENFKSDLCDIPACDSKDSKEKNKM
EILY
SEQ ID NO: 31; mouse ROR1
MHRPRRRGTRPPPLALLAALLLAARGADAQETELSVSAELVPTSSWNTSSEIDKGSY
LTLDEPMNNITTSLGQTAELHCKVSGNPPPSIRWFKNDAPVVQEPRRISFRATNYGSR
LRIRNLDTTDTGYFQCVATNGKKVVSTTGVLFVKFGPPPTASPGSSDEYEEDGFCQP
YRGIACARFIGNRTVYMESLHMQGEIENQITAAFTMIGTSSHLSDKCSQFAIPSLCHY
AFPYCDETSSVPKPRDLCRDECEVLENVLCQTEYIFARSNPMILMRLKLPNCEDLPQP
ESPEAANCIRIGIPMADPINKNHKCYNSTGVDYRGTVSVTKSGRQCQPWNSQYPHTH
SFTALRFPELNGGHSYCRNPGNQKEAPWCFTLDENFKSDLCDIPACDSKDSKEKNKM
EILYILVPSVAIPLAIAFLFFFICVCRNNQKSSSPPVQRQPKPVRGQNVEMSMLNAYKP
KSKAKELPLSAVRFMEELGECTFGKIYKGHLYLPGMDHAQLVAIKTLKDYNNPQQW
TEFQQEASLMAELHHPNIVCLLGAVTQEQPVCMLFEYMNQGDLHEFLIMRSPHSDV
GCSSDEDGTVKSSLDHGDFLHIAIQIAAGMEYLSSHFFVHKDLAARNILIGEQLHVKIS
DLGLSREIYSADYYRVQSKSSLPIRWMPPEAIMYGKFSSDSDIWSFGVVLWEIFSFGL
QPYYGFSNQEVIEMVRKRQLLPCSEDCPPRMYSLMTECWNEIPSRRPRFKDIHVRLRS
WEGLSSHTSSTTPSGGNATTQTTSLSASPVSNLSNPRFPNYMFPSQGITPQGQIAGFIG
PAIPQNQRFIPINGYPIPPGYAAFPAAHYQPAGPPRVIQHCPPPKSRSPSSASGSTSTGH
VASLPSSGSNQEANVPLLPHMSIPNHPGGMGITVFGNKSQKPYKIDSKQSSLLGDSHI
HGHTESMISAEV
SEQ ID NO: 32; PRT; Human ROR1 Ig domain epitope
LDEPMNNITTSLGQTAELHCKVSGNPPPTIRWFKNDAPVVQEPRRLSFRSTIYGSRLRI
RNLDTTDTGYFQCVATNGKEVVSSTGVLFVKFGPPPTASPGY
SEQ ID NO: 33; PRT; Human ROR1 Ig subdomain epitope
VSGNPPPTIRWFKNDAPVVQEPRRLSFRSTIYGSRLRIRNLDTTDTGYFQCVATNGKE
VVSSTGVLFVKFGPPPTASPGY
SEQ ID NO: 34; PRT; Human ROR1 Kringle domain epitope
HKCYNSTGVDYRGTVSVTKSGRQCQPWNSQYPHTHTFTALRFPELNGGHSYCRNPG
NQKEAPWCFTLDENFKSDLCDIPACDS
SEQ ID NO: 35; PRT; UC-101 FR H1
EVQLLETGGGLVQPGGSRKLSCVAS
SEQ ID NO: 36; PRT; UC-101 CDR H1
GFSFSSYG
SEQ ID NO: 37; PRT; UC-101 FR H2
MHWVRQAPEKGLEWVAY
SEQ ID NO: 38; PRT; UC-101 CDR H2
IGGTSNTI
SEQ ID NO: 39; PRT; UC-101 FR H3
YFADTVKGRFTISRDNAKNTLFLQMASLRSEDTAMYYC
SEQ ID NO: 40; PRT; UC-101 CDR H3
ARTNLLGMDY
SEQ ID NO: 41; PRT; UC-101 FR H4
WGQGTSVTVSS
SEQ ID NO: 42; PRT; UC-101 FR LI
QIVLTQSPTIMSASPGEKVTMTCSAS
SEQ ID NO: 43; PRT; UC-101 CDR L1
SSVSD
SEQ ID NO: 44; PRT; UC-101 FR L2
MYWYQQKPGSSPRLLIY
SEQ ID NO: 45; PRT; UC-101 CDR L2
DTS
SEQ ID NO: 46; PRT; UC-101 FR L3
NLASGVPVRFSGSGSGTSYSLTISRMEAEDAATYYC
SEQ ID NO: 47; PRT; UC-101 CDR L3
QQWISYT
SEQ ID NO: 48; PRT; UC-101 FR L4
FGGGTKLEIKR
SEQ ID NO: 49; PRT; UC-106 FR H1
QIQLVQSGPELKKPGETVKISCKAS
SEQ ID NO: 50; PRT; UC-106 CDR H1
GYTFTYYP
SEQ ID NO: 51; PRT; UC-106 FR H2
IHWVKQAPGKGLKWMGW
SEQ ID NO: 52; PRT; UC-106 CDR H2
INTYSGVP
SEQ ID NO: 53; PRT; UC-106 FR H3
TYADDFKGRFAFSLETSANTAYLQINNLRNEDMATYFC
SEQ ID NO: 54; PRT; UC-106 CDR H3
ARGDSSLFDY
SEQ ID NO: 55; PRT; UC-106 FR H4
WGQGTTLTVSS
SEQ ID NO: 56; PRT; UC-106 FR L1
GHQMTQSPSSLSASLGERVSLTCRAS
SEQ ID NO: 57; PRT; UC-106 CDR L1
QEISGY
SEQ ID NO: 58; PRT; UC-106 FR L2
LIWLQQKPDGTIKRLIY
SEQ ID NO: 59; PRT; UC-106 CDR L2
AAS
SEQ ID NO: 60; PRT; UC-106 FR L3
TLNSGVPKRFSGSRSGSDYSLTISSLESEDFADYYC
SEQ ID NO: 61; PRT; UC-106 CDR L3
LQYTSYPLT
SEQ ID NO: 62; PRT; UC-106 FR L4
FGAGTKLELKR
SEQ ID NO: 63; PRT; UC-102 FR H1
DVHLQESGPGLVKPSQSLSVTCTVT
SEQ ID NO: 64; PRT; UC-102 CDR H1
GYSITGSYS
SEQ ID NO: 65; PRT; UC-102 FR H2
WNWIRQFPGNKLEWMGY
SEQ ID NO: 66; PRT; UC-102 CDR H2
IHSSGTT
SEQ ID NO: 67; PRT; UC-102 FR H3
NYNPSLKSRISFTRDTSKNQLFLQLNSVTTEDTATYYC
SEQ ID NO: 68; PRT; UC-102 CDR H3
TRGFAY
SEQ ID NO: 69; PRT; UC-102 FR H4
WGQGTLVTVSA
SEQ ID NO: 70; PRT; UC-102 FR LI
NIVMTQSPSSMSASLGDRITITCQAT
SEQ ID NO: 71; PRT; UC-102 CDR L1
QDIVKN
SEQ ID NO: 72; PRT; UC-102 FR L2
LNWYQQKPGKPPSFLIY
SEQ ID NO: 73; PRT; UC-102 CDR L2
YTT
SEQ ID NO: 74; PRT; UC-102 FR L3
ELAEGVPSRFSGSGSGSDYSLTISNLESEDFADYYC
SEQ ID NO: 75; PRT; UC-102 CDR L3
LQFYEFPPT
SEQ ID NO: 76; PRT; UC-102 FR L4
FGAGTKLELKR
SEQ ID NO: 77; PRT; UC-103 FR H1
EIQLQQSGPVLVKPGASVKVSCKAS
SEQ ID NO: 78; PRT; UC-103 CDR H1
GYAFTAYN
SEQ ID NO: 79; PRT; UC-103 FR H2
IHWVRQSHGKRLEWIGS
SEQ ID NO: 80; PRT; UC-103 CDR H2
FDPYDGGS
SEQ ID NO: 81; PRT; UC-103 FR H3
SYNQKFKDKATLTVDKSSTTAYMHLNSLTSEDSAVYYC
SEQ ID NO: 82; PRT; UC-103 CDR H3
ARGWYYFDY
SEQ ID NO: 83; PRT; UC-103 FR H4
WGHGTTLTVSS
SEQ ID NO: 84; PRT; UC-103 FR L1
DVQITQSPSYLAASPGETITINCRAS
SEQ ID NO: 85; PRT; UC-103 CDR L1
KSISKY
SEQ ID NO: 86; PRT; UC-103 FR L2
LAWYQEKPGKTNKLLIY
SEQ ID NO: 87; PRT; UC-103 CDR L2
SGS
SEQ ID NO: 88; PRT; UC-103 FR L3
TLQSGIPSRFRGSGSGTDFTLTISSLEPEDFAMYYC
SEQ ID NO: 89; PRT; UC-103 CDR L3
QQHDESPYT
SEQ ID NO: 90; PRT; UC-103 FR L4
FGEGTKLEIKR
SEQ ID NO: 91; PRT; UC-104 FR H1
EVQLLETGGGLVQPGGSRKLSCAAS
SEQ ID NO: 92; PRT; UC-104 CDR H1
GFTFSNYG
SEQ ID NO: 93; PRT; UC-104 FR H2
MHWVRQAPGKGLEWVAH
SEQ ID NO: 94; PRT; UC-104 CDR H2
ISRYSDTI
SEQ ID NO: 95; PRT; UC-104 FR H3
YYADAVKGRFTISRDNAKNTLFLQMTTLRSEDTAIYYC
SEQ ID NO: 96; PRT; UC-104 CDR H3
TSAMDY
SEQ ID NO: 97; PRT; UC-104 FR H4
WGQGTSVTVSS
SEQ ID NO: 98; PRT; UC-104 FR L1
DIQMTQTTSSLSASLGDRVTISCRAS
SEQ ID NO: 99; PRT; UC-104 CDR L1
QDISNY
SEQ ID NO: 100; PRT; UC-104 FR L2
LNWFQQKPDGTIKLLIY
SEQ ID NO: 101; PRT; UC-104 CDR L2
YTS
SEQ ID NO: 102; PRT; UC-104 FR L3
RLHSGVPSRFSGSGSGTDYSLTISNLEQEDIATYFC
SEQ ID NO: 103; PRT; UC-104 CDR L3
QQGNTLPFT
SEQ ID NO: 104; PRT; UC-104 FR L4
FGSGTKLEIKR
SEQ ID NO: 105; PRT; UC-105 FR H1
EVKLVESGGGLVQPGGSRKLSCAAS
SEQ ID NO: 106; PRT; UC-105 CDR H1
GFTFSSYG
SEQ ID NO: 107; PRT; UC-105 FR H2
MHWIRQTPDKGLEWVAY
SEQ ID NO: 108; PRT; UC-105 CDR H2
ISSNSDHI
SEQ ID NO: 109; PRT; UC-105 FR H3
FYTDTVKGRFTISRDNAKNTLFLQMTSLRSEDTAMYYC
SEQ ID NO: 110; PRT; UC-105 CDR H3
ASRMDY
SEQ ID NO: 111; PRT; UC-105 V FR H4
WGQGTSVTVSS
SEQ ID NO: 112; PRT; UC-105 FR L1
DIQMTQTTSSLSASLGDRVTISCRAS
SEQ ID NO: 113; PRT; UC-105 CDR L1
QDISNH
SEQ ID NO: 114; PRT; UC-105 FR L2
LNWYQQKPDGTVKLLIY
SEQ ID NO: 115; PRT; UC-105 CDR L2
YTS
SEQ ID NO: 116; PRT; UC-105 FR L3
RLHSGVPSRFSGSGSGTDYSLTISNLEQGDIATYFC
SEQ ID NO: 117; PRT; UC-105 CDR L3
QQGSTLPYT
SEQ ID NO: 118; PRT; UC-105 FR L4
FGGGTKLEIKR
SEQ ID NO: 119; PRT; UC-107 FR H1
QIQLVQSGPELKKPGETVKISCKAS
SEQ ID NO: 120; PRT; UC-107 CDR H1
GYTFTNYP
SEQ ID NO: 121; PRT; UC-107 FR H2
IHWVKQAPGKGLKWMGW
SEQ ID NO: 122; PRT; UC-107 CDR H2
INTYSGVP
SEQ ID NO: 123; PRT; UC-107 FR H3
TYADDFKGRFAFSLETSANTAYLQLNNLRNEDMATYFC
SEQ ID NO: 124; PRT; UC-107 CDR H3
ARGDSSLFDY
SEQ ID NO: 125 PRT; UC-107 FR H4
WGQGTTLTVSS
SEQ ID NO: 126; PRT; UC-107 FR L1
DVQMIQSPSSLSASLGERVSLTCRAS
SEQ ID NO: 127; PRT; UC-107 CDR L1
QEISGY
SEQ ID NO: 128; PRT; UC-107 FR L2
LIWLQQKPDGTITRLIY
SEQ ID NO: 129; PRT; UC-107 CDR L2
AAS
SEQ ID NO: 130; PRT; UC-107 FR L3
TLDSGVPKRFSGSRSGSDYSLTISSLESEDFADYYC
SEQ ID NO: 131 DNA; UC-107 CDR L3
LQYTSYPLT
SEQ ID NO: 132 DNA; UC-107 FR L4
FGAGTKLELKR
SEQ ID NO: 133; DNA; UC-101 FR H1
GAAGTGCAGCTGTTGGAGACTGGGGGAGGCTTAGTGCAGCCTGGAGGGTCCCGG
AAACTCTCCTGTGTAGCCTCT
SEQ ID NO: 134; DNA; UC-101 CDR H1
GGATTCAGTTTCAGTAGCTATGGA
SEQ ID NO: 135; DNA; UC-101 FR H2
ATGCACTGGGTTCGTCAGGCTCCAGAGAAGGGGCTGGAGTGGGTCGCATAT
SEQ ID NO: 136; DNA; UC-101 CDR H2
ATTGGTGGTACCAGTAATACCATC
SEQ ID NO: 137; DNA; UC-101 FR H3
TACTTTGCAGACACAGTGAAGGGCCGATTCACCATTTCCAGAGACAATGCCAAG
AACACCCTGTTCCTGCAAATGGCCAGTCTAAGGTCTGAGGACACGGCCATGTATT
ACTGT
SEQ ID NO: 138; DNA; UC-101 CDR H3
GCAAGAACGAACTTACTCGGGATGGACTAC
SEQ ID NO: 139; DNA; UC-101 FR H4
TGGGGTCAAGGAACCTCAGTCACCGTCTCCTCA
SEQ ID NO: 140; DNA; UC-101 FR L1
CAAATTGTTCTCACCCAGTCTCCAACAATCATGTCTGCATCTCCAGGGGAGAAGG
TCACCATGACCTGCAGTGCCAGC
SEQ ID NO: 141; DNA; UC-101 CDR L1
TCAAGTGTAAGTGAC
SEQ ID NO: 142; DNA; UC-101 FR L2
ATGTACTGGTACCAGCAGAAGCCAGGATCCTCCCCCAGACTCCTCATTTAT
SEQ ID NO: 143; DNA; UC-101 CDR L2
GACACATCC
SEQ ID NO: 144; DNA; UC-101 FR L3
AACCTGGCTTCTGGAGTCCCTGTTCGCTTCAGTGGCAGTGGGTCTGGGACCTCTT
ACTCTCTCACAATCAGCCGAATGGAGGCTGAAGATGCTGCCACTTATTACTGC
SEQ ID NO: 145; DNA; UC-101 CDR L3
CAGCAGTGGATTAGTTACACG
SEQ ID NO: 146; DNA; UC-101 FR L4
TTCGGAGGGGGGACCAAGCTGGAAATAAAACGG
SEQ ID NO: 147; DNA; UC-102 FR H1
GATGTGCACCTTCAGGAGTCTGGACCTGGCCTGGTGAAACCTTCTCAGTCTCTAT
CTGTCACCTGCACTGTCACT
SEQ ID NO: 148; DNA; UC-102 CDR H1
GGCTACTCCATCACCGGTAGTTATAGC
SEQ ID NO: 149; DNA; UC-102 FR H2
TGGAACTGGATCCGGCAGTTTCCAGGAAACAAACTGGAGTGGATGGGCTAC
SEQ ID NO: 150; DNA; UC-102 CDR H2
ATACACTCCAGTGGTACCACT
SEQ ID NO: 151; DNA; UC-102 FR H3
AACTACAACCCATCTCTCAAAAGTCGAATCTCTTTTACTCGAGACACATCCAAGA
ACCAACTCTTCCTGCAGTTGAATTCTGTGACTACTGAGGACACAGCCACATATTA
CTGT
SEQ ID NO: 152; DNA; UC-102 CDR H3
ACAAGGGGGTTTGCTTAC
SEQ ID NO: 153; DNA; UC-102 FR H4
TGGGGCCAAGGGACTCTGGTCACTGTCTCTGCA
SEQ ID NO: 154; DNA; UC-102 FR L1
AACATCGTTATGACCCAGTCTCCATCCTCTATGTCTGCATCTCTGGGAGACAGAA
TAACCATCACTTGCCAGGCAACT
SEQ ID NO: 155; DNA; UC-102 CDR L1
CAAGACATTGTTAAGAAT
SEQ ID NO: 156; DNA; UC-102 FR L2
TTAAACTGGTATCAGCAGAAACCAGGGAAACCCCCTTCATTCCTGATCTAT
SEQ ID NO: 157; DNA; UC-102 CDR L2
TATACAACT
SEQ ID NO: 158; DNA; UC-102 FR L3
GAACTGGCAGAAGGGGTCCCATCAAGGTTCAGTGGCAGTGGGTCTGGGTCAGAC
TATTCTCTGACAATCAGCAACCTGGAGTCTGAAGATTTTGCAGACTATTACTGT
SEQ ID NO: 159; DNA; UC-102 CDR L3
CTTCAGTTTTATGAGTTTCCTCCCACG
SEQ ID NO: 160; DNA; UC-102 FR L4
TTCGGTGCTGGGACCAAGCTGGAGCTGAAACGG
SEQ ID NO: 161; DNA; UC-103 FR H1
GAGATCCAGCTGCAGCAGTCTGGACCTGTCCTGGTGAAGCCTGGGGCTTCAGTG
AAGGTTTCTTGCAAGGCTTCT
SEQ ID NO: 162; DNA; UC-103 CDR H1
GGTTATGCATTCACTGCCTACAAC
SEQ ID NO: 163; DNA; UC-103 FR H2
ATACACTGGGTGAGACAGAGCCATGGAAAGCGCCTTGAGTGGATTGGATCT
SEQ ID NO: 164; DNA; UC-103 CDR H2
TTTGATCCTTACGATGGTGGTAGT
SEQ ID NO: 165; DNA; UC-103 FR H3
AGTTACAACCAGAAGTTCAAGGACAAAGCCACATTGACTGTAGACAAATCTTCC
ACCACAGCCTACATGCATCTCAACAGCCTGACATCTGAGGACTCTGCAGTCTATT
ACTGT
SEQ ID NO: 166; DNA; UC-103 CDR H3
GCAAGGGGGTGGTACTACTTTGACTAC
SEQ ID NO: 167; DNA; UC-103 FR H4
TGGGGCCACGGGACCACTCTCACAGTCTCCTCA
SEQ ID NO: 168; DNA; UC-103 FR L1
GACGTCCAGATAACCCAGTCTCCATCTTATCTTGCTGCATCTCCTGGAGAAACCA
TTACTATTAATTGCAGGGCAAGT
SEQ ID NO: 169; DNA; UC-103 CDR L1
AAGAGCATTAGCAAATAT
SEQ ID NO: 170; DNA; UC-103 FR L2
TTAGCCTGGTATCAAGAGAAACCTGGGAAAACTAATAAGCTCCTTATCTAC
SEQ ID NO: 171; DNA; UC-103 CDR L2
TCTGGATCC
SEQ ID NO: 172; DNA; UC-103 FR L3
ACTTTGCAATCTGGAATTCCATCAAGATTCAGGGGCAGTGGATCTGGTACAGATT
TCACTCTCACCATCAGTAGCCTGGAGCCTGAAGATTTTGCAATGTATTACTGT
SEQ ID NO: 173; DNA; UC-103 CDR L3
CAACAGCATGATGAATCCCCGTACACG
SEQ ID NO: 174; DNA; UC-103 FR L4
TTCGGAGAGGGGACCAAGCTGGAAATAAAACGG
SEQ ID NO: 175; DNA; UC-104 FR H1
GAAGTGCAGCTGTTGGAGACTGGGGGAGGCTTAGTGCAGCCTGGAGGGTCCCGG
AAACTCTCCTGTGCAGCCTCT
SEQ ID NO: 176; DNA; UC-104 CDR H1
GGATTCACTTTCAGTAACTATGGA
SEQ ID NO: 177; DNA; UC-104 FR H2
ATGCACTGGGTTCGTCAGGCTCCAGGGAAGGGGCTGGAGTGGGTCGCACAT
SEQ ID NO: 178; DNA; UC-104 CDR H2
ATTAGTCGTTACAGTGATACCATC
SEQ ID NO: 179; DNA; UC-104 FR H3
TACTATGCAGACGCAGTGAAGGGCCGATTCACCATCTCCAGAGACAATGCCAAG
AACACCCTGTTCCTGCAAATGACCACTCTAAGGTCTGAGGACACGGCCATATATT
ACTGT
SEQ ID NO: 180; DNA; UC-104 CDR H3
ACAAGTGCTATGGACTAC
SEQ ID NO: 181; DNA; UC-104 FR H4
TGGGGTCAAGGAACCTCAGTCACCGTCTCCTCA
SEQ ID NO: 182; DNA; UC-104 FR L1
GATATCCAGATGACACAGACTACATCCTCCCTGTCTGCCTCTCTGGGAGACAGAG
TCACCATCAGTTGCAGGGCAAGT
SEQ ID NO: 183; DNA; UC-104 CDR L1
CAGGACATTAGCAATTAT
SEQ ID NO: 184; DNA; UC-104 FR L2
TTAAACTGGTTTCAGCAGAAACCAGATGGAACTATTAAACTCCTGATCTAC
SEQ ID NO: 185; DNA; UC-104 CDR L2
TACACATCA
SEQ ID NO: 186; DNA; UC-104 FR L3
AGATTACACTCAGGAGTCCCATCAAGGTTCAGTGGCAGTGGGTCTGGAACAGAT
TATTCTCTCACCATTAGCAACCTGGAACAAGAAGATATTGCCACTTACTTTTGC
SEQ ID NO: 187; DNA; UC-104 CDR L3
CAGCAGGGTAATACGCTTCCATTCACG
SEQ ID NO: 188; DNA; UC-104 FR L4
TTCGGCTCGGGGACAAAGTTGGAAATAAAACGG
SEQ ID NO: 189; DNA; UC-105 FR H1
GAGGTGAAGCTGGTGGAATCTGGGGGAGGCTTAGTGCAGCCTGGAGGGTCCCGG
AAACTCTCCTGTGCAGCCTCT
SEQ ID NO: 190; DNA; UC-105 CDR H1
GGATTCACTTTCAGTAGCTATGGA
SEQ ID NO: 191; DNA; UC-105 FR H2
ATGCACTGGATTCGTCAGACTCCAGACAAGGGGCTGGAATGGGTCGCATAT
SEQ ID NO: 192; DNA; UC-105 CDR H2
ATTAGTAGTAACAGTGATCACATC
SEQ ID NO: 193; DNA; UC-105 FR H3
TTCTATACAGACACAGTGAAGGGCCGATTCACCATCTCCAGAGACAATGCCAAG
AACACCCTGTTCCTGCAAATGACCAGTCTAAGGTCTGAGGACACGGCCATGTATT
ACTGT
SEQ ID NO: 194; DNA; UC-105 CDR H3
GCAAGCCGTATGGACTAC
SEQ ID NO: 195; DNA; UC-105 FR H4
TGGGGTCAAGGAACCTCAGTCACCGTCTCCTCA
SEQ ID NO: 196; DNA; UC-105 FR L1
GATATCCAGATGACACAGACTACATCCTCCCTGTCTGCCTCTCTGGGAGACAGAG
TCACCATCAGTTGCAGGGCAAGT
SEQ ID NO: 197; DNA; UC-105 CDR L1
CAGGACATTAGCAATCAT
SEQ ID NO: 198; DNA; UC-105 FR L2
TTAAACTGGTATCAGCAGAAACCAGATGGAACTGTTAAACTCCTGATCTAC
SEQ ID NO: 199; DNA; UC-105 CDR L2
TACACATCA
SEQ ID NO: 200; DNA; UC-105 FR L3
AGATTACACTCAGGAGTCCCATCAAGGTTCAGTGGCAGTGGGTCTGGAACAGAT
TATTCTCTCACCATTAGCAACCTGGAGCAAGGCGATATTGCCACTTACTTTTGC
SEQ ID NO: 201; DNA; UC-105 CDR L3
CAACAGGGTAGTACGCTTCCGTACACA
SEQ ID NO: 202; DNA; UC-105 FR L4
TTCGGAGGGGGGACCAAGCTGGAAATAAAACGG
SEQ ID NO: 203; DNA; UC-106 FR H1
CAGATCCAGTTGGTGCAATCTGGACCTGAGCTGAAGAAGCCTGGAGAGACAGTC
AAGATCTCCTGCAAGGCTTCT
SEQ ID NO: 204; DNA; UC-106 CDR H1
GGGTATACCTTCACATACTATCCA
SEQ ID NO: 205; DNA; UC-106 FR H2
ATCCACTGGGTGAAGCAGGCTCCAGGAAAGGGTTTAAAGTGGATGGGCTGG
SEQ ID NO: 206; DNA; UC-106 CDR H2
ATAAACACCTACTCTGGAGTGCCA
SEQ ID NO: 207; DNA; UC-106 FR H3
ACATATGCAGATGACTTCAAGGGACGGTTTGCCTTCTCTTTGGAAACCTCTGCCA
ACACTGCATATTTGCAGATCAACAACCTCAGAAATGAAGACATGGCTACATATTT
CTGT
SEQ ID NO: 208; DNA; UC-106 CDR H3
GCAAGGGGGGATAGTAGCCTTTTTGACTAC
SEQ ID NO: 209; DNA; UC-106 FR H4
TGGGGCCAAGGCACCACTCTCACAGTCTCCTCA
SEQ ID NO: 210; DNA; UC-106 FR L1
GGACATCAAATGACCCAGTCTCCATCCTCCTTATCTGCCTCTCTGGGAGAAAGAG
TCAGTCTCACTTGTCGGGCAAGT
SEQ ID NO: 211; DNA; UC-106 CDR L1
CAGGAAATTAGTGGTTAC
SEQ ID NO: 212; DNA; UC-106 FR L2
TTAATCTGGCTTCAGCAGAAACCGGATGGAACTATTAAACGCCTGATCTAC
SEQ ID NO: 213; DNA; UC-106 CDR L2
GCCGCATCC
SEQ ID NO: 214; DNA; UC-106 FR L3
ACTTTAAATTCTGGTGTCCCAAAAAGGTTCAGTGGCAGTAGGTCTGGGTCAGATT
ACTCTCTCACCATCAGCAGCCTTGAGTCTGAAGATTTTGCTGACTATTACTGT
SEQ ID NO: 215; DNA; UC-106 CDR L3
CTACAATATACTAGTTATCCTCTCACG
SEQ ID NO: 216; DNA; UC-106 FR L4
TTCGGTGCTGGGACCAAGCTGGAGCTGAAACGG
SEQ ID NO: 217; DNA; UC-107 FR H1
CAGATCCAGTTGGTGCAATCTGGACCTGAGCTGAAGAAGCCTGGAGAGACAGTC
AAGATCTCCTGCAAGGCTTCT
SEQ ID NO: 218; DNA; UC-107 CDR H1
GGATATACCTTCACAAACTATCCA
SEQ ID NO: 219; DNA; UC-107 FR H2
ATACACTGGGTGAAGCAGGCTCCAGGAAAGGGTTTAAAGTGGATGGGCTGG
SEQ ID NO: 220; DNA; UC-107 CDR H2
ATAAACACCTACTCTGGAGTGCCA
SEQ ID NO: 221; DNA; UC-107 FR H3
ACATATGCAGATGACTTCAAGGGACGGTTTGCCTTCTCTTTGGAAACCTCTGCCA
ACACTGCATATTTGCAGCTCAACAACCTCAGAAATGAGGACATGGCTACATATTT
CTGT
SEQ ID NO: 222; DNA; UC-107 CDR H3
GCAAGGGGGGATAGTAGCCTTTTTGACTAC
SEQ ID NO: 223; DNA; UC-107 FR H4
TGGGGCCAAGGCACCACTCTCACAGTCTCCTCA
SEQ ID NO: 224; DNA; UC-107 FR L1
GACGTTCAGATGATCCAGTCTCCATCCTCCTTATCTGCCTCTCTGGGAGAAAGAG
TCAGTCTCACTTGTCGGGCAAGT
SEQ ID NO: 225; DNA; UC-107 CDR L1
CAGGAAATTAGTGGTTAC
SEQ ID NO: 226; DNA; UC-107 FR L2
TTAATCTGGCTTCAGCAGAAACCAGATGGAACTATTACACGCCTGATCTAC
SEQ ID NO: 227; DNA; UC-107 CDR L2
GCCGCATCC
SEQ ID NO: 228; DNA; UC-107 FR L3
ACTTTAGATTCTGGTGTCCCAAAAAGGTTCAGTGGCAGTAGGTCTGGGTCAGATT
ACTCTCTCACCATCAGCAGCCTTGAGTCTGAAGATTTTGCTGACTATTACTGT
SEQ ID NO: 229; DNA; UC-107 CDR L3
CTACAATATACTAGTTATCCTCTCACG
SEQ ID NO: 230; DNA; UC-107 FR L4
TTCGGTGCTGGGACCAAGCTGGAGCTGAAACGG

What is claimed is:

1. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:36, a CDR H2 as set forth in SEQ ID NO:38, and a CDR H3 as set forth in SEQ ID NO:40; and
wherein said light chain variable domain comprises:
a CDR L1 as set forth in SEQ ID NO:43, a CDR L2 as set forth in SEQ ID NO:45 and a CDR L3 as set forth in SEQ ID NO:47.

2. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:64, a CDR H2 as set forth in SEQ ID NO:66, and a CDR H3 as set forth in SEQ ID NO:68; and
wherein said light chain variable domain comprises:
a CDR L1 as set forth in SEQ ID NO:71, a CDR L2 as set forth in SEQ ID NO:73 and a CDR L3 as set forth in SEQ ID NO:75.

3. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:78, a CDR H2 as set forth in SEQ ID NO:80, and a CDR H3 as set forth in SEQ ID NO:82; and
wherein said light chain variable domain comprises:
a CDR L1 as set forth in SEQ ID NO:85, a CDR L2 as set forth in SEQ ID NO:87 and a CDR L3 as set forth in SEQ ID NO: 89.

4. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:92, a CDR H2 as set forth in SEQ ID NO:94, and a CDR H3 as set forth in SEQ ID NO:96; and
wherein said light chain variable domain comprises:
a CDR L1 as set forth in SEQ ID NO:99, a CDR L2 as set forth in SEQ ID NO:101 and a CDR L3 as set forth in SEQ ID NO:103.

5. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:106, a CDR H2 as set forth in SEQ ID NO: 108, and a CDR H3 as set forth in SEQ ID NO:110; and
wherein said light chain variable domain comprises:
a CDR L1 as set forth in SEQ ID NO: 113, a CDR L2 as set forth in SEQ ID NO:115 and a CDR L3 as set forth in SEQ ID NO: 117.

6. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:50, a CDR H2 as set forth in SEQ ID NO:52, and a CDR H3 as set forth in SEQ ID NO:54; and
wherein said light chain variable domain comprises:
a CDR L1 as set forth in SEQ ID NO:57, a CDR L2 as set forth in SEQ ID NO:59 and a CDR L3 as set forth in SEQ ID NO:61.

7. An anti-tyrosine kinase-like orphan receptor 1 (ROR1) antibody comprising a heavy chain variable domain and a light chain variable domain,
wherein said heavy chain variable domain comprises a CDR H1 as set forth in SEQ ID NO:120, a CDR H2 as set forth in SEQ ID NO: 122, and a CDR H3 as set forth in SEQ ID NO:124; and
wherein said light chain variable domain comprises:
a CDR L1 as set forth in SEQ ID NO:127, a CDR L2 as set forth in SEQ ID NO:129 and a CDR L3 as set forth in SEQ ID NO:131.

8. The anti-ROR1 antibody of any one of claims 1-7, wherein said antibody is a humanized antibody, a chimeric antibody, a Fab′ fragment or a scFv.

9. The anti-ROR1 antibody of claim 8, wherein said antibody is a humanized antibody.

10. The anti-ROR1 antibody of any one of claims 1-7, wherein said heavy chain variable domain comprises the sequence of SEQ ID NO:2, SEQ ID NO:6, SEQ ID NO:10, SEQ ID NO:14, SEQ ID NO:18, SEQ ID NO:22 or SEQ ID NO:26.

11. The anti-ROR1 antibody of any one of claims 1-7, wherein said light chain variable domain comprises the sequence of SEQ ID NO:4, SEQ ID NO:8, SEQ ID NO:12, SEQ ID NO:16, SEQ ID NO:20, SEQ ID NO:24 or SEQ ID NO:28.

12. The anti-ROR1 antibody of claim 1, wherein said heavy chain variable domain comprises the sequence of SEQ ID NO:2 and said light chain variable domain comprises the sequence of SEQ ID NO:4.

13. The anti-ROR1 antibody of claim 2, wherein said heavy chain variable domain comprises the sequence of SEQ ID NO:6 and said light chain variable domain comprises the sequence of SEQ ID NO:8.

14. The anti-ROR1 antibody of claim 3, wherein said heavy chain variable domain comprises the sequence of SEQ ID NO:10 and said light chain variable domain comprises the sequence of SEQ ID NO: 12.

15. The anti-ROR1 antibody of claim 4, wherein said heavy chain variable domain comprises the sequence of SEQ ID NO:14 and said light chain variable domain comprises the sequence of SEQ ID NO: 16.

16. The anti-ROR1 antibody of claim 5, wherein said heavy chain variable domain comprises the sequence of SEQ ID NO:18 and said light chain variable domain comprises the sequence of SEQ ID NO:20.

17. The anti-ROR1 antibody of claim 6, wherein said heavy chain variable domain comprises the sequence of SEQ ID NO:22 and said light chain variable domain comprises the sequence of SEQ ID NO:24.

18. The anti-ROR1 antibody of claim 7, wherein said heavy chain variable domain comprises the sequence of SEQ ID NO:26 and said light chain variable domain comprises the sequence of SEQ ID NO:28.

19. The anti-ROR1 antibody of any one of claims 1-5, wherein said anti-ROR1 antibody binds the Ig-like domain of human ROR1.

20. The anti-ROR1 antibody of any one of claims 6-7, wherein said anti-ROR1 antibody binds the Kringle domain of human ROR1.

21. The anti-ROR1 antibody of any one of claims 4-5, wherein said anti-ROR1 antibody binds a ROR1 polypeptide comprising a glutamic acid at a position corresponding to position 138 of SEQ ID NO:30.

22. The anti-ROR1 antibody of claim 6 or 17, wherein said anti-ROR1 antibody binds a ROR1 polypeptide comprising a threonine at a position corresponding to position 346 of SEQ ID NO:30.

23. An anti-ROR1 antibody, wherein said anti-ROR1 antibody binds the same epitope as an antibody comprising: a heavy chain variable domain comprising a CDR H1 as set forth in SEQ ID NO:36, a CDR H2 as set forth in SEQ ID NO:38 and a CDR H3 as set forth in SEQ ID NO:40; and a light chain variable domain comprising a CDR L1 as set forth in SEQ ID NO:43, a CDR L2 as set forth in SEQ ID NO:45, and a CDR L3 as set forth in SEQ ID NO:47.

24. An anti-ROR1 antibody, wherein said anti-ROR1 antibody binds the same epitope as an antibody comprising: a heavy chain variable domain comprising a CDR H1 as set forth in SEQ ID NO:64, a CDR H2 as set forth in SEQ ID NO:66 and a CDR H3 as set forth in SEQ ID NO:68; and a light chain variable domain comprising a CDR L1 as set forth in SEQ ID NO:71, a CDR L2 as set forth in SEQ ID NO:73, and a CDR L3 as set forth in SEQ ID NO:75.

25. An anti-ROR1 antibody, wherein said anti-ROR1 antibody binds the same epitope as an antibody comprising: a heavy chain variable domain comprising a CDR H1 as set forth in SEQ ID NO:78, a CDR H2 as set forth in SEQ ID NO:80 and a CDR H3 as set forth in SEQ ID NO:82; and a light chain variable domain comprising a CDR L1 as set forth in SEQ ID NO: 85, a CDR L2 as set forth in SEQ ID NO:87, and a CDR L3 as set forth in SEQ ID NO:89.

26. An anti-ROR1 antibody, wherein said anti-ROR1 antibody binds the same epitope as an antibody comprising: a heavy chain variable domain comprising a CDR H1 as set forth in SEQ ID NO:92, a CDR H2 as set forth in SEQ ID NO:94 and a CDR H3 as set forth in SEQ ID NO:96; and a light chain variable domain comprising a CDR L1 as set forth in SEQ ID NO:99, a CDR L2 as set forth in SEQ ID NO:101, and a CDR L3 as set forth in SEQ ID NO: 103.

27. An anti-ROR1 antibody, wherein said anti-ROR1 antibody binds the same epitope as an antibody comprising: a heavy chain variable domain comprising a CDR H1 as set forth in SEQ ID NO: 106, a CDR H2 as set forth in SEQ ID NO:108 and a CDR H3 as set forth in SEQ ID NO:110; and a light chain variable domain comprising a CDR L1 as set forth in SEQ ID NO: 113, a CDR L2 as set forth in SEQ ID NO:115, and a CDR L3 as set forth in SEQ ID NO:117.

28. An anti-ROR1 antibody, wherein said anti-ROR1 antibody binds the same epitope as an antibody comprising: a heavy chain variable domain comprising a CDR H1 as set forth in SEQ ID NO:50, a CDR H2 as set forth in SEQ ID NO:52 and a CDR H3 as set forth in SEQ ID NO:54; and a light chain variable domain comprising a CDR L1 as set forth in SEQ ID NO:57, a CDR L2 as set forth in SEQ ID NO:59, and a CDR L3 as set forth in SEQ ID NO:61.

29. An anti-ROR1 antibody, wherein said anti-ROR1 antibody binds the same epitope as an antibody comprising: a heavy chain variable domain comprising a CDR H1 as set forth in SEQ ID NO: 120, a CDR H2 as set forth in SEQ ID NO:122 and a CDR H3 as set forth in SEQ ID NO:124; and a light chain variable domain comprising a CDR L1 as set forth in SEQ ID NO: 127, a CDR L2 as set forth in SEQ ID NO: 129, and a CDR L3 as set forth in SEQ ID NO:131.

30. The anti-ROR1 antibody of any one of claims 1-7 or 23-29, wherein said anti-ROR1 antibody is bound to a ROR1 protein.

31. The anti-ROR1 antibody of claim 30, wherein said ROR1 protein forms part of a cell.

32. The anti-ROR1 antibody of claim 30, wherein said ROR1 protein is expressed on the surface of a cell.

33. The anti-ROR1 antibody of claim 31, wherein said cell is a cancer cell.

34. The anti-ROR1 antibody of claim 33, wherein said cancer cell is a B cell leukemia cell, a mantle cell lymphoma (MCL) cell, a Burkett's Lymphoma cell, a lymphoma cell, a chronic lymphocytic leukemia (CLL) cell, an Acute Myeloid Leukemia (AML) cell, a B-Cell Acute Lymphoblastic Leukemia (B-ALL) cell, a T-cell acute lymphoblastic leukemia (T-ALL) cell, a renal cancer cell, a colon cancer cell, a breast cancer cell, an ovarian cancer cell, a lung cancer cell, a skin cancer cell, a pancreatic cancer cell, a testicular cancer cell, a bladder cancer cell, a uterine cancer cell, a prostate cancer cell, or an adrenal cancer cell.

35. The anti-ROR1 antibody of any one of claims 1-7 or 23-29, wherein said anti-ROR1 antibody is bound to a therapeutic moiety or a diagnostic moiety.

36. A cell comprising an anti-ROR1 antibody of any one of claims 1-7 or 23-29.

37. A nucleic acid encoding an anti-ROR1 antibody of any one of claims 1-7 or 23-29.

38. A pharmaceutical composition comprising a therapeutically effective amount of an anti-ROR1 antibody of any one of claims 1-7 or 23-29, and a pharmaceutically acceptable excipient.

39. A method of treating cancer in a subject in need thereof, said method comprising administering to a subject a therapeutically effective amount of an anti-ROR1 antibody of any one of claims 1-35 or 23-29.

40. The method of claim 39, wherein said cancer is B cell leukemia, mantle cell lymphoma (MCL), Burkett's Lymphoma, lymphoma, chronic lymphocytic leukemia (CLL), Acute Myeloid Leukemia (AML), B-Cell Acute Lymphoblastic Leukemia (B-ALL), T-cell acute lymphoblastic leukemia (T-ALL), renal cancer, colon cancer, breast cancer, ovarian cancer, lung cancer, skin cancer, pancreatic cancer, testicular cancer, bladder cancer, uterine cancer, prostate cancer, or adrenal cancer.

41. A method of identifying an anti-ROR1 antibody, the method comprising:
(i) contacting an antibody with a first ROR1 polypeptide comprising a threonine at a position corresponding to position 346 of SEQ ID NO:30;
(ii) detecting said antibody binding to said first ROR1 polypeptide;
(iii) contacting said antibody with a second ROR 1 polypeptide not comprising a threonine at a position corresponding to position 346 of SEQ ID NO:30; and
(iv) detecting said antibody not binding to said second ROR1 polypeptide, thereby identifying an anti-ROR1 antibody.

42. The method of claim 41, wherein said second ROR1 polypeptide comprises a serine at a position corresponding to position 346 of SEQ ID NO:30.

43. The method of claim 41, wherein said first ROR1 polypeptide is a first truncated ROR1 polypeptide.

44. The method of claim 43, wherein said first truncated ROR1 polypeptide comprises amino acid residues 311-393 of the sequence of SEQ ID NO:30.

45. The method of claim 43, wherein said second ROR1 polypeptide is a second truncated ROR1 polypeptide.

46. The method of claim 45, wherein said second truncated ROR1 polypeptide comprises amino acid residues 311-393 of the sequence of SEQ ID NO:31.

47. The method of claim 41, wherein said antibody is a humanized antibody.

48. The method of claim 41, wherein said antibody is an antibody fragment.

49. The method of claim 41, wherein said antibody is a chimeric antibody.

50. The method of claim 41, wherein said antibody is a single chain antibody.


SEQUENCE LISTING (230 sequences)

SEQ ID NO: 1 - DNA, 351 bp, Artificial Sequence
  Synthetic Polynucleotide
gaagtgcagctgttggagactgggggaggcttagtgcagcctggagggtcccggaaactctcctgtgtagcctctggattcagtttcagtagctatggaatgcactgggttcgtcaggctccagagaaggggctggagtgggtcgcatatattggtggtaccagtaataccatctactttgcagacacagtgaagggccgattcaccatttccagagacaatgccaagaacaccctgttcctgcaaatggccagtctaaggtctgaggacacggccatgtattactgtgcaagaacgaacttactcgggatggactactggggtcaaggaacctcagtcaccgtctcctca

SEQ ID NO: 2 - protein, 117 aa, Artificial Sequence
  Synthetic Polypeptide
EVQLLETGGGLVQPGGSRKLSCVASGFSFSSYGMHWVRQAPEKGLEWVAYIGGTSNTIYFADTVKGRFTISRDNAKNTLFLQMASLRSEDTAMYYCARTNLLGMDYWGQGTSVTVSS

SEQ ID NO: 3 - DNA, 315 bp, Artificial Sequence
  Synthetic Polynucleotide
caaattgttctcacccagtctccaacaatcatgtctgcatctccaggggagaaggtcaccatgacctgcagtgccagctcaagtgtaagtgacatgtactggtaccagcagaagccaggatcctcccccagactcctcatttatgacacatccaacctggcttctggagtccctgttcgcttcagtggcagtgggtctgggacctcttactctctcacaatcagccgaatggaggctgaagatgctgccacttattactgccagcagtggattagttacacgttcggaggggggaccaagctggaaataaaacgg

SEQ ID NO: 4 - protein, 105 aa, Artificial Sequence
  Synthetic Polypeptide
QIVLTQSPTIMSASPGEKVTMTCSASSSVSDMYWYQQKPGSSPRLLIYDTSNLASGVPVRFSGSGSGTSYSLTISRMEAEDAATYYCQQWISYTFGGGTKLEIKR

SEQ ID NO: 5 - DNA, 339 bp, Artificial Sequence
  Synthetic Polynucleotide
gatgtgcaccttcaggagtctggacctggcctggtgaaaccttctcagtctctatctgtcacctgcactgtcactggctactccatcaccggtagttatagctggaactggatccggcagtttccaggaaacaaactggagtggatgggctacatacactccagtggtaccactaactacaacccatctctcaaaagtcgaatctcttttactcgagacacatccaagaaccaactcttcctgcagttgaattctgtgactactgaggacacagccacatattactgtacaagggggtttgcttactggggccaagggactctggtcactgtctctgca

SEQ ID NO: 6 - protein, 113 aa, Artificial Sequence
  Synthetic Polypeptide
DVHLQESGPGLVKPSQSLSVTCTVTGYSITGSYSWNWIRQFPGNKLEWMGYIHSSGTTNYNPSLKSRISFTRDTSKNQLFLQLNSVTTEDTATYYCTRGFAYWGQGTLVTVSA

SEQ ID NO: 7 - DNA, 324 bp, Artificial Sequence
  Synthetic Polynucleotide
aacatcgttatgacccagtctccatcctctatgtctgcatctctgggagacagaataaccatcacttgccaggcaactcaagacattgttaagaatttaaactggtatcagcagaaaccagggaaacccccttcattcctgatctattatacaactgaactggcagaaggggtcccatcaaggttcagtggcagtgggtctgggtcagactattctctgacaatcagcaacctggagtctgaagattttgcagactattactgtcttcagttttatgagtttcctcccacgttcggtgctgggaccaagctggagctgaaacgg

SEQ ID NO: 8 - protein, 108 aa, Artificial Sequence
  Synthetic Polypeptide
NIVMTQSPSSMSASLGDRITITCQATQDIVKNLNWYQQKPGKPPSFLIYYTTELAEGVPSRFSGSGSGSDYSLTISNLESEDFADYYCLQFYEFPPTFGAGTKLELKR

SEQ ID NO: 9 - DNA, 348 bp, Artificial Sequence
  Synthetic Polynucleotide
gagatccagctgcagcagtctggacctgtcctggtgaagcctggggcttcagtgaaggtttcttgcaaggcttctggttatgcattcactgcctacaacatacactgggtgagacagagccatggaaagcgccttgagtggattggatcttttgatccttacgatggtggtagtagttacaaccagaagttcaaggacaaagccacattgactgtagacaaatcttccaccacagcctacatgcatctcaacagcctgacatctgaggactctgcagtctattactgtgcaagggggtggtactactttgactactggggccacgggaccactctcacagtctcctca

SEQ ID NO: 10 - protein, 116 aa, Artificial Sequence
  Synthetic Polypeptide
EIQLQQSGPVLVKPGASVKVSCKASGYAFTAYNIHWVRQSHGKRLEWIGSFDPYDGGSSYNQKFKDKATLTVDKSSTTAYMHLNSLTSEDSAVYYCARGWYYFDYWGHGTTLTVSS

SEQ ID NO: 11 - DNA, 324 bp, Artificial Sequence
  Synthetic Polynucleotide
gacgtccagataacccagtctccatcttatcttgctgcatctcctggagaaaccattactattaattgcagggcaagtaagagcattagcaaatatttagcctggtatcaagagaaacctgggaaaactaataagctccttatctactctggatccactttgcaatctggaattccatcaagattcaggggcagtggatctggtacagatttcactctcaccatcagtagcctggagcctgaagattttgcaatgtattactgtcaacagcatgatgaatccccgtacacgttcggagaggggaccaagctggaaataaaacgg

SEQ ID NO: 12 - protein, 108 aa, Artificial Sequence
  Synthetic Polypeptide
DVQITQSPSYLAASPGETITINCRASKSISKYLAWYQEKPGKTNKLLIYSGSTLQSGIPSRFRGSGSGTDFTLTISSLEPEDFAMYYCQQHDESPYTFGEGTKLEIKR

SEQ ID NO: 13 - DNA, 339 bp, Artificial Sequence
  Synthetic Polynucleotide
gaagtgcagctgttggagactgggggaggcttagtgcagcctggagggtcccggaaactctcctgtgcagcctctggattcactttcagtaactatggaatgcactgggttcgtcaggctccagggaaggggctggagtgggtcgcacatattagtcgttacagtgataccatctactatgcagacgcagtgaagggccgattcaccatctccagagacaatgccaagaacaccctgttcctgcaaatgaccactctaaggtctgaggacacggccatatattactgtacaagtgctatggactactggggtcaaggaacctcagtcaccgtctcctca

SEQ ID NO: 14 - protein, 113 aa, Artificial Sequence
  Synthetic Polypeptide
EVQLLETGGGLVQPGGSRKLSCAASGFTFSNYGMHWVRQAPGKGLEWVAHISRYSDTIYYADAVKGRFTISRDNAKNTLFLQMTTLRSEDTAIYYCTSAMDYWGQGTSVTVSS

SEQ ID NO: 15 - DNA, 324 bp, Artificial Sequence
  Synthetic Polynucleotide
gatatccagatgacacagactacatcctccctgtctgcctctctgggagacagagtcaccatcagttgcagggcaagtcaggacattagcaattatttaaactggtttcagcagaaaccagatggaactattaaactcctgatctactacacatcaagattacactcaggagtcccatcaaggttcagtggcagtgggtctggaacagattattctctcaccattagcaacctggaacaagaagatattgccacttacttttgccagcagggtaatacgcttccattcacgttcggctcggggacaaagttggaaataaaacgg

SEQ ID NO: 16 - protein, 108 aa, Artificial Sequence
  Synthetic Polypeptide
DIQMTQTTSSLSASLGDRVTISCRASQDISNYLNWFQQKPDGTIKLLIYYTSRLHSGVPSRFSGSGSGTDYSLTISNLEQEDIATYFCQQGNTLPFTFGSGTKLEIKR

SEQ ID NO: 17 - DNA, 339 bp, Artificial Sequence
  Synthetic Polynucleotide
gaggtgaagctggtggaatctgggggaggcttagtgcagcctggagggtcccggaaactctcctgtgcagcctctggattcactttcagtagctatggaatgcactggattcgtcagactccagacaaggggctggaatgggtcgcatatattagtagtaacagtgatcacatcttctatacagacacagtgaagggccgattcaccatctccagagacaatgccaagaacaccctgttcctgcaaatgaccagtctaaggtctgaggacacggccatgtattactgtgcaagccgtatggactactggggtcaaggaacctcagtcaccgtctcctca

SEQ ID NO: 18 - protein, 113 aa, Artificial Sequence
  Synthetic Polypeptide
EVKLVESGGGLVQPGGSRKLSCAASGFTFSSYGMHWIRQTPDKGLEWVAYISSNSDHIFYTDTVKGRFTISRDNAKNTLFLQMTSLRSEDTAMYYCASRMDYWGQGTSVTVSS

SEQ ID NO: 19 - DNA, 324 bp, Artificial Sequence
  Synthetic Polynucleotide
gatatccagatgacacagactacatcctccctgtctgcctctctgggagacagagtcaccatcagttgcagggcaagtcaggacattagcaatcatttaaactggtatcagcagaaaccagatggaactgttaaactcctgatctactacacatcaagattacactcaggagtcccatcaaggttcagtggcagtgggtctggaacagattattctctcaccattagcaacctggagcaaggcgatattgccacttacttttgccaacagggtagtacgcttccgtacacattcggaggggggaccaagctggaaataaaacgg

SEQ ID NO: 20 - protein, 108 aa, Artificial Sequence
  Synthetic Polypeptide
DIQMTQTTSSLSASLGDRVTISCRASQDISNHLNWYQQKPDGTVKLLIYYTSRLHSGVPSRFSGSGSGTDYSLTISNLEQGDIATYFCQQGSTLPYTFGGGTKLEIKR

SEQ ID NO: 21 - DNA, 351 bp, Artificial Sequence
  Synthetic Polynucleotide
cagatccagttggtgcaatctggacctgagctgaagaagcctggagagacagtcaagatctcctgcaaggcttctgggtataccttcacatactatccaatccactgggtgaagcaggctccaggaaagggtttaaagtggatgggctggataaacacctactctggagtgccaacatatgcagatgacttcaagggacggtttgccttctctttggaaacctctgccaacactgcatatttgcagatcaacaacctcagaaatgaagacatggctacatatttctgtgcaaggggggatagtagcctttttgactactggggccaaggcaccactctcacagtctcctca

SEQ ID NO: 22 - protein, 117 aa, Artificial Sequence
  Synthetic Polypeptide
QIQLVQSGPELKKPGETVKISCKASGYTFTYYPIHWVKQAPGKGLKWMGWINTYSGVPTYADDFKGRFAFSLETSANTAYLQINNLRNEDMATYFCARGDSSLFDYWGQGTTLTVSS

SEQ ID NO: 23 - DNA, 324 bp, Artificial Sequence
  Synthetic Polynucleotide
ggacatcaaatgacccagtctccatcctccttatctgcctctctgggagaaagagtcagtctcacttgtcgggcaagtcaggaaattagtggttacttaatctggcttcagcagaaaccggatggaactattaaacgcctgatctacgccgcatccactttaaattctggtgtcccaaaaaggttcagtggcagtaggtctgggtcagattactctctcaccatcagcagccttgagtctgaagattttgctgactattactgtctacaatatactagttatcctctcacgttcggtgctgggaccaagctggagctgaaacgg

SEQ ID NO: 24 - protein, 108 aa, Artificial Sequence
  Synthetic Polypeptide
GHQMTQSPSSLSASLGERVSLTCRASQEISGYLIWLQQKPDGTIKRLIYAASTLNSGVPKRFSGSRSGSDYSLTISSLESEDFADYYCLQYTSYPLTFGAGTKLELKR

SEQ ID NO: 25 - DNA, 351 bp, Artificial Sequence
  Synthetic Polynucleotide
cagatccagttggtgcaatctggacctgagctgaagaagcctggagagacagtcaagatctcctgcaaggcttctggatataccttcacaaactatccaatacactgggtgaagcaggctccaggaaagggtttaaagtggatgggctggataaacacctactctggagtgccaacatatgcagatgacttcaagggacggtttgccttctctttggaaacctctgccaacactgcatatttgcagctcaacaacctcagaaatgaggacatggctacatatttctgtgcaaggggggatagtagcctttttgactactggggccaaggcaccactctcacagtctcctca

SEQ ID NO: 26 - protein, 117 aa, Artificial Sequence
  Synthetic Polypeptide
QIQLVQSGPELKKPGETVKISCKASGYTFTNYPIHWVKQAPGKGLKWMGWINTYSGVPTYADDFKGRFAFSLETSANTAYLQLNNLRNEDMATYFCARGDSSLFDYWGQGTTLTVSS

SEQ ID NO: 27 - DNA, 324 bp, Artificial Sequence
  Synthetic Polynucleotide
gacgttcagatgatccagtctccatcctccttatctgcctctctgggagaaagagtcagtctcacttgtcgggcaagtcaggaaattagtggttacttaatctggcttcagcagaaaccagatggaactattacacgcctgatctacgccgcatccactttagattctggtgtcccaaaaaggttcagtggcagtaggtctgggtcagattactctctcaccatcagcagccttgagtctgaagattttgctgactattactgtctacaatatactagttatcctctcacgttcggtgctgggaccaagctggagctgaaacgg

SEQ ID NO: 28 - protein, 108 aa, Artificial Sequence
  Synthetic Polypeptide
DVQMIQSPSSLSASLGERVSLTCRASQEISGYLIWLQQKPDGTITRLIYAASTLDSGVPKRFSGSRSGSDYSLTISSLESEDFADYYCLQYTSYPLTFGAGTKLELKR

SEQ ID NO: 29 - protein, 937 aa, Artificial Sequence
  Synthetic Polypeptide
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 30 - protein, 406 aa, Artificial Sequence
  Synthetic Polypeptide
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 31 - protein, 937 aa, Artificial Sequence
  Synthetic Polypeptide
MHRPRRRGTRPPPLALLAALLLAARGADAQETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEILYILVPSVAIPLAIAFLFFFICVCRNNQKSSSPPVQRQPKPVRGQNVEMSMLNAYKPKSKAKELPLSAVRFMEELGECTFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSSLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRFPNYMFPSQGITPQGQIAGFIGPAIPQNQRFIPINGYPIPPGYAAFPAAHYQPAGPPRVIQHCPPPKSRSPSSASGSTSTGHVASLPSSGSNQEANVPLLPHMSIPNHPGGMGITVFGNKSQKPYKIDSKQSSLLGDSHIHGHTESMISAEV

SEQ ID NO: 32 - protein, 101 aa, Artificial Sequence
  Synthetic Polypeptide
LDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGY

SEQ ID NO: 33 - protein, 80 aa, Artificial Sequence
  Synthetic Polypeptide
VSGNPPPTIRWFKNDAPVVQEPRRLSFRSTIYGSRLRIRNLDTTDTGYFQCVATNGKEVVSSTGVLFVKFGPPPTASPGY

SEQ ID NO: 34 - protein, 83 aa, Artificial Sequence
  Synthetic Polypeptide
HKCYNSTGVDYRGTVSVTKSGRQCQPWNSQYPHTHTFTALRFPELNGGHSYCRNPGNQKEAPWCFTLDENFKSDLCDIPACDS

SEQ ID NO: 35 - protein, 25 aa, Artificial Sequence
  Synthetic Polypeptide
EVQLLETGGGLVQPGGSRKLSCVAS

SEQ ID NO: 36 - protein, 8 aa, Artificial Sequence
  Synthetic Polypeptide
GFSFSSYG

SEQ ID NO: 37 - protein, 17 aa, Artificial Sequence
  Synthetic Polypeptide
MHWVRQAPEKGLEWVAY

SEQ ID NO: 38 - protein, 8 aa, Artificial Sequence
  Synthetic Polypeptide
IGGTSNTI

SEQ ID NO: 39 - protein, 38 aa, Artificial Sequence
  Synthetic Polypeptide
YFADTVKGRFTISRDNAKNTLFLQMASLRSEDTAMYYC

SEQ ID NO: 40 - protein, 10 aa, Artificial Sequence
  Synthetic Polypeptide
ARTNLLGMDY

SEQ ID NO: 41 - protein, 11 aa, Artificial Sequence
  Synthetic Polypeptide
WGQGTSVTVSS

SEQ ID NO: 42 - protein, 26 aa, Artificial Sequence
  Synthetic Polypeptide
QIVLTQSPTIMSASPGEKVTMTCSAS

SEQ ID NO: 43 - protein, 5 aa, Artificial Sequence
  Synthetic Polypeptide
SSVSD

SEQ ID NO: 44 - protein, 17 aa, Artificial Sequence
  Synthetic Polypeptide
MYWYQQKPGSSPRLLIY

SEQ ID NO: 45 - protein, 3 aa, Artificial Sequence
  Synthetic Polypeptide
DTS

SEQ ID NO: 46 - protein, 36 aa, Artificial Sequence
  Synthetic Polypeptide
NLASGVPVRFSGSGSGTSYSLTISRMEAEDAATYYC

SEQ ID NO: 47 - protein, 7 aa, Artificial Sequence
  Synthetic Polypeptide
QQWISYT

SEQ ID NO: 48 - protein, 11 aa, Artificial Sequence
  Synthetic Polypeptide
FGGGTKLEIKR

SEQ ID NO: 49 - protein, 25 aa, Artificial Sequence
  Synthetic Polypeptide
QIQLVQSGPELKKPGETVKISCKAS

SEQ ID NO: 50 - protein, 8 aa, Artificial Sequence
  Synthetic Polypeptide
GYTFTYYP

SEQ ID NO: 51 - protein, 17 aa, Artificial Sequence
  Synthetic Polypeptide
IHWVKQAPGKGLKWMGW

SEQ ID NO: 52 - protein, 8 aa, Artificial Sequence
  Synthetic Polypeptide
INTYSGVP

SEQ ID NO: 53 - protein, 38 aa, Artificial Sequence
  Synthetic Polypeptide
TYADDFKGRFAFSLETSANTAYLQINNLRNEDMATYFC

SEQ ID NO: 54 - protein, 10 aa, Artificial Sequence
  Synthetic Polypeptide
ARGDSSLFDY

SEQ ID NO: 55 - protein, 11 aa, Artificial Sequence
  Synthetic Polypeptide
WGQGTTLTVSS

SEQ ID NO: 56 - protein, 26 aa, Artificial Sequence
  Synthetic Polypeptide
GHQMTQSPSSLSASLGERVSLTCRAS

SEQ ID NO: 57 - protein, 6 aa, Artificial Sequence
  Synthetic Polypeptide
QEISGY

SEQ ID NO: 58 - protein, 17 aa, Artificial Sequence
  Synthetic Polypeptide
LIWLQQKPDGTIKRLIY

SEQ ID NO: 59 - protein, 3 aa, Artificial Sequence
  Synthetic Polypeptide
AAS

SEQ ID NO: 60 - protein, 36 aa, Artificial Sequence
  Synthetic Polypeptide
TLNSGVPKRFSGSRSGSDYSLTISSLESEDFADYYC

SEQ ID NO: 61 - protein, 9 aa, Artificial Sequence
  Synthetic Polypeptide
LQYTSYPLT

SEQ ID NO: 62 - protein, 11 aa, Artificial Sequence
  Synthetic Polypeptide
FGAGTKLELKR

SEQ ID NO: 63 - protein, 25 aa, Artificial Sequence
  Synthetic Polypeptide
DVHLQESGPGLVKPSQSLSVTCTVT

SEQ ID NO: 64 - protein, 9 aa, Artificial Sequence
  Synthetic Polypeptide
GYSITGSYS

SEQ ID NO: 65 - protein, 17 aa, Artificial Sequence
  Synthetic Polypeptide
WNWIRQFPGNKLEWMGY

SEQ ID NO: 66 - protein, 7 aa, Artificial Sequence
  Synthetic Polypeptide
IHSSGTT

SEQ ID NO: 67 - protein, 38 aa, Artificial Sequence
  Synthetic Polypeptide
NYNPSLKSRISFTRDTSKNQLFLQLNSVTTEDTATYYC

SEQ ID NO: 68 - protein, 6 aa, Artificial Sequence
  Synthetic Polypeptide
TRGFAY

SEQ ID NO: 69 - protein, 11 aa, Artificial Sequence
  Synthetic Polypeptide
WGQGTLVTVSA

SEQ ID NO: 70 - protein, 26 aa, Artificial Sequence
  Synthetic Polypeptide
NIVMTQSPSSMSASLGDRITITCQAT

SEQ ID NO: 71 - protein, 6 aa, Artificial Sequence
  Synthetic Polypeptide
QDIVKN

SEQ ID NO: 72 - protein, 17 aa, Artificial Sequence
  Synthetic Polypeptide
LNWYQQKPGKPPSFLIY

SEQ ID NO: 73 - protein, 3 aa, Artificial Sequence
  Synthetic Polypeptide
YTT

SEQ ID NO: 74 - protein, 36 aa, Artificial Sequence
  Synthetic Polypeptide
ELAEGVPSRFSGSGSGSDYSLTISNLESEDFADYYC

SEQ ID NO: 75 - protein, 9 aa, Artificial Sequence
  Synthetic Polypeptide
LQFYEFPPT

SEQ ID NO: 76 - protein, 11 aa, Artificial Sequence
  Synthetic Polypeptide
FGAGTKLELKR

SEQ ID NO: 77 - protein, 25 aa, Artificial Sequence
  Synthetic Polypeptide
EIQLQQSGPVLVKPGASVKVSCKAS

SEQ ID NO: 78 - protein, 8 aa, Artificial Sequence
  Synthetic Polypeptide
GYAFTAYN

SEQ ID NO: 79 - protein, 17 aa, Artificial Sequence
  Synthetic Polypeptide
IHWVRQSHGKRLEWIGS

SEQ ID NO: 80 - protein, 8 aa, Artificial Sequence
  Synthetic Polypeptide
FDPYDGGS

SEQ ID NO: 81 - protein, 38 aa, Artificial Sequence
  Synthetic Polypeptide
SYNQKFKDKATLTVDKSSTTAYMHLNSLTSEDSAVYYC

SEQ ID NO: 82 - protein, 9 aa, Artificial Sequence
  Synthetic Polypeptide
ARGWYYFDY

SEQ ID NO: 83 - protein, 11 aa, Artificial Sequence
  Synthetic Polypeptide
WGHGTTLTVSS

SEQ ID NO: 84 - protein, 26 aa, Artificial Sequence
  Synthetic Polypeptide
DVQITQSPSYLAASPGETITINCRAS

SEQ ID NO: 85 - protein, 6 aa, Artificial Sequence
  Synthetic Polypeptide
KSISKY

SEQ ID NO: 86 - protein, 17 aa, Artificial Sequence
  Synthetic Polypeptide
LAWYQEKPGKTNKLLIY

SEQ ID NO: 87 - protein, 3 aa, Artificial Sequence
  Synthetic Polypeptide
SGS

SEQ ID NO: 88 - protein, 36 aa, Artificial Sequence
  Synthetic Polypeptide
TLQSGIPSRFRGSGSGTDFTLTISSLEPEDFAMYYC

SEQ ID NO: 89 - protein, 9 aa, Artificial Sequence
  Synthetic Polypeptide
QQHDESPYT

SEQ ID NO: 90 - protein, 11 aa, Artificial Sequence
  Synthetic Polypeptide
FGEGTKLEIKR

SEQ ID NO: 91 - protein, 25 aa, Artificial Sequence
  Synthetic Polypeptide
EVQLLETGGGLVQPGGSRKLSCAAS

SEQ ID NO: 92 - protein, 8 aa, Artificial Sequence
  Synthetic Polypeptide
GFTFSNYG

SEQ ID NO: 93 - protein, 17 aa, Artificial Sequence
  Synthetic Polypeptide
MHWVRQAPGKGLEWVAH

SEQ ID NO: 94 - protein, 8 aa, Artificial Sequence
  Synthetic Polypeptide
ISRYSDTI

SEQ ID NO: 95 - protein, 38 aa, Artificial Sequence
  Synthetic Polypeptide
YYADAVKGRFTISRDNAKNTLFLQMTTLRSEDTAIYYC

SEQ ID NO: 96 - protein, 6 aa, Artificial Sequence
  Synthetic Polypeptide
TSAMDY

SEQ ID NO: 97 - protein, 11 aa, Artificial Sequence
  Synthetic Polypeptide
WGQGTSVTVSS

SEQ ID NO: 98 - protein, 26 aa, Artificial Sequence
  Synthetic Polypeptide
DIQMTQTTSSLSASLGDRVTISCRAS

SEQ ID NO: 99 - protein, 6 aa, Artificial Sequence
  Synthetic Polypeptide
QDISNY

SEQ ID NO: 100 - protein, 17 aa, Artificial Sequence
  Synthetic Polypeptide
LNWFQQKPDGTIKLLIY

SEQ ID NO: 101 - protein, 3 aa, Artificial Sequence
  Synthetic Polypeptide
YTS

SEQ ID NO: 102 - protein, 36 aa, Artificial Sequence
  Synthetic Polypeptide
RLHSGVPSRFSGSGSGTDYSLTISNLEQEDIATYFC

SEQ ID NO: 103 - protein, 9 aa, Artificial Sequence
  Synthetic Polypeptide
QQGNTLPFT

SEQ ID NO: 104 - protein, 11 aa, Artificial Sequence
  Synthetic Polypeptide
FGSGTKLEIKR

SEQ ID NO: 105 - protein, 25 aa, Artificial Sequence
  Synthetic Polypeptide
EVKLVESGGGLVQPGGSRKLSCAAS

SEQ ID NO: 106 - protein, 8 aa, Artificial Sequence
  Synthetic Polypeptide
GFTFSSYG

SEQ ID NO: 107 - protein, 17 aa, Artificial Sequence
  Synthetic Polypeptide
MHWIRQTPDKGLEWVAY

SEQ ID NO: 108 - protein, 8 aa, Artificial Sequence
  Synthetic Polypeptide
ISSNSDHI

SEQ ID NO: 109 - protein, 38 aa, Artificial Sequence
  Synthetic Polypeptide
FYTDTVKGRFTISRDNAKNTLFLQMTSLRSEDTAMYYC

SEQ ID NO: 110 - protein, 6 aa, Artificial Sequence
  Synthetic Polypeptide
ASRMDY

SEQ ID NO: 111 - protein, 11 aa, Artificial Sequence
  Synthetic Polypeptide
WGQGTSVTVSS

SEQ ID NO: 112 - protein, 26 aa, Artificial Sequence
  Synthetic Polypeptide
DIQMTQTTSSLSASLGDRVTISCRAS

SEQ ID NO: 113 - protein, 6 aa, Artificial Sequence
  Synthetic Polypeptide
QDISNH

SEQ ID NO: 114 - protein, 17 aa, Artificial Sequence
  Synthetic Polypeptide
LNWYQQKPDGTVKLLIY

SEQ ID NO: 115 - protein, 3 aa, Artificial Sequence
  Synthetic Polypeptide
YTS

SEQ ID NO: 116 - protein, 36 aa, Artificial Sequence
  Synthetic Polypeptide
RLHSGVPSRFSGSGSGTDYSLTISNLEQGDIATYFC

SEQ ID NO: 117 - protein, 9 aa, Artificial Sequence
  Synthetic Polypeptide
QQGSTLPYT

SEQ ID NO: 118 - protein, 11 aa, Artificial Sequence
  Synthetic Polypeptide
FGGGTKLEIKR

SEQ ID NO: 119 - protein, 25 aa, Artificial Sequence
  Synthetic Polypeptide
QIQLVQSGPELKKPGETVKISCKAS

SEQ ID NO: 120 - protein, 8 aa, Artificial Sequence
  Synthetic Polypeptide
GYTFTNYP

SEQ ID NO: 121 - protein, 17 aa, Artificial Sequence
  Synthetic Polypeptide
IHWVKQAPGKGLKWMGW

SEQ ID NO: 122 - protein, 8 aa, Artificial Sequence
  Synthetic Polypeptide
INTYSGVP

SEQ ID NO: 123 - protein, 38 aa, Artificial Sequence
  Synthetic Polypeptide
TYADDFKGRFAFSLETSANTAYLQLNNLRNEDMATYFC

SEQ ID NO: 124 - protein, 10 aa, Artificial Sequence
  Synthetic Polypeptide
ARGDSSLFDY

SEQ ID NO: 125 - protein, 11 aa, Artificial Sequence
  Synthetic Polypeptide
WGQGTTLTVSS

SEQ ID NO: 126 - protein, 26 aa, Artificial Sequence
  Synthetic Polypeptide
DVQMIQSPSSLSASLGERVSLTCRAS

SEQ ID NO: 127 - protein, 6 aa, Artificial Sequence
  Synthetic Polypeptide
QEISGY

SEQ ID NO: 128 - protein, 17 aa, Artificial Sequence
  Synthetic Polypeptide
LIWLQQKPDGTITRLIY

SEQ ID NO: 129 - protein, 3 aa, Artificial Sequence
  Synthetic Polypeptide
AAS

SEQ ID NO: 130 - protein, 36 aa, Artificial Sequence
  Synthetic Polypeptide
TLDSGVPKRFSGSRSGSDYSLTISSLESEDFADYYC

SEQ ID NO: 131 - protein, 9 aa, Artificial Sequence
  Synthetic Polypeptide
LQYTSYPLT

SEQ ID NO: 132 - protein, 11 aa, Artificial Sequence
  Synthetic Polypeptide
FGAGTKLELKR

SEQ ID NO: 133 - DNA, 75 bp, Artificial Sequence
  Synthetic Polynucleotide
gaagtgcagctgttggagactgggggaggcttagtgcagcctggagggtcccggaaactctcctgtgtagcctct

SEQ ID NO: 134 - DNA, 24 bp, Artificial Sequence
  Synthetic Polynucleotide
ggattcagtttcagtagctatgga

SEQ ID NO: 135 - DNA, 51 bp, Artificial Sequence
  Synthetic Polynucleotide
atgcactgggttcgtcaggctccagagaaggggctggagtgggtcgcatat

SEQ ID NO: 136 - DNA, 24 bp, Artificial Sequence
  Synthetic Polynucleotide
attggtggtaccagtaataccatc

SEQ ID NO: 137 - DNA, 114 bp, Artificial Sequence
  Synthetic Polynucleotide
tactttgcagacacagtgaagggccgattcaccatttccagagacaatgccaagaacaccctgttcctgcaaatggccagtctaaggtctgaggacacggccatgtattactgt

SEQ ID NO: 138 - DNA, 30 bp, Artificial Sequence
  Synthetic Polynucleotide
gcaagaacgaacttactcgggatggactac

SEQ ID NO: 139 - DNA, 33 bp, Artificial Sequence
  Synthetic Polynucleotide
tggggtcaaggaacctcagtcaccgtctcctca

SEQ ID NO: 140 - DNA, 78 bp, Artificial Sequence
  Synthetic Polynucleotide
caaattgttctcacccagtctccaacaatcatgtctgcatctccaggggagaaggtcaccatgacctgcagtgccagc

SEQ ID NO: 141 - DNA, 15 bp, Artificial Sequence
  Synthetic Polynucleotide
tcaagtgtaagtgac

SEQ ID NO: 142 - DNA, 51 bp, Artificial Sequence
  Synthetic Polynucleotide
atgtactggtaccagcagaagccaggatcctcccccagactcctcatttat

SEQ ID NO: 143 - DNA, 9 bp, Artificial Sequence
  Synthetic Polynucleotide
gacacatcc

SEQ ID NO: 144 - DNA, 108 bp, Artificial Sequence
  Synthetic Polynucleotide
aacctggcttctggagtccctgttcgcttcagtggcagtgggtctgggacctcttactctctcacaatcagccgaatggaggctgaagatgctgccacttattactgc

SEQ ID NO: 145 - DNA, 21 bp, Artificial Sequence
  Synthetic Polynucleotide
cagcagtggattagttacacg

SEQ ID NO: 146 - DNA, 33 bp, Artificial Sequence
  Synthetic Polynucleotide
ttcggaggggggaccaagctggaaataaaacgg

SEQ ID NO: 147 - DNA, 75 bp, Artificial Sequence
  Synthetic Polynucleotide
gatgtgcaccttcaggagtctggacctggcctggtgaaaccttctcagtctctatctgtcacctgcactgtcact

SEQ ID NO: 148 - DNA, 27 bp, Artificial Sequence
  Synthetic Polynucleotide
ggctactccatcaccggtagttatagc

SEQ ID NO: 149 - DNA, 51 bp, Artificial Sequence
  Synthetic Polynucleotide
tggaactggatccggcagtttccaggaaacaaactggagtggatgggctac

SEQ ID NO: 150 - DNA, 21 bp, Artificial Sequence
  Synthetic Polynucleotide
atacactccagtggtaccact

SEQ ID NO: 151 - DNA, 114 bp, Artificial Sequence
  Synthetic Polynucleotide
aactacaacccatctctcaaaagtcgaatctcttttactcgagacacatccaagaaccaactcttcctgcagttgaattctgtgactactgaggacacagccacatattactgt

SEQ ID NO: 152 - DNA, 18 bp, Artificial Sequence
  Synthetic Polynucleotide
acaagggggtttgcttac

SEQ ID NO: 153 - DNA, 33 bp, Artificial Sequence
  Synthetic Polynucleotide
tggggccaagggactctggtcactgtctctgca

SEQ ID NO: 154 - DNA, 78 bp, Artificial Sequence
  Synthetic Polynucleotide
aacatcgttatgacccagtctccatcctctatgtctgcatctctgggagacagaataaccatcacttgccaggcaact

SEQ ID NO: 155 - DNA, 18 bp, Artificial Sequence
  Synthetic Polynucleotide
caagacattgttaagaat

SEQ ID NO: 156 - DNA, 51 bp, Artificial Sequence
  Synthetic Polynucleotide
ttaaactggtatcagcagaaaccagggaaacccccttcattcctgatctat

SEQ ID NO: 157 - DNA, 9 bp, Artificial Sequence
  Synthetic Polynucleotide
tatacaact

SEQ ID NO: 158 - DNA, 108 bp, Artificial Sequence
  Synthetic Polynucleotide
gaactggcagaaggggtcccatcaaggttcagtggcagtgggtctgggtcagactattctctgacaatcagcaacctggagtctgaagattttgcagactattactgt

SEQ ID NO: 159 - DNA, 27 bp, Artificial Sequence
  Synthetic Polynucleotide
cttcagttttatgagtttcctcccacg

SEQ ID NO: 160 - DNA, 33 bp, Artificial Sequence
  Synthetic Polynucleotide
ttcggtgctgggaccaagctggagctgaaacgg

SEQ ID NO: 161 - DNA, 75 bp, Artificial Sequence
  Synthetic Polynucleotide
gagatccagctgcagcagtctggacctgtcctggtgaagcctggggcttcagtgaaggtttcttgcaaggcttct

SEQ ID NO: 162 - DNA, 24 bp, Artificial Sequence
  Synthetic Polynucleotide
ggttatgcattcactgcctacaac

SEQ ID NO: 163 - DNA, 51 bp, Artificial Sequence
  Synthetic Polynucleotide
atacactgggtgagacagagccatggaaagcgccttgagtggattggatct

SEQ ID NO: 164 - DNA, 24 bp, Artificial Sequence
  Synthetic Polynucleotide
tttgatccttacgatggtggtagt

SEQ ID NO: 165 - DNA, 114 bp, Artificial Sequence
  Synthetic Polynucleotide
agttacaaccagaagttcaaggacaaagccacattgactgtagacaaatcttccaccacagcctacatgcatctcaacagcctgacatctgaggactctgcagtctattactgt

SEQ ID NO: 166 - DNA, 27 bp, Artificial Sequence
  Synthetic Polynucleotide
gcaagggggtggtactactttgactac

SEQ ID NO: 167 - DNA, 33 bp, Artificial Sequence
  Synthetic Polynucleotide
tggggccacgggaccactctcacagtctcctca

SEQ ID NO: 168 - DNA, 78 bp, Artificial Sequence
  Synthetic Polynucleotide
gacgtccagataacccagtctccatcttatcttgctgcatctcctggagaaaccattactattaattgcagggcaagt

SEQ ID NO: 169 - DNA, 18 bp, Artificial Sequence
  Synthetic Polynucleotide
aagagcattagcaaatat

SEQ ID NO: 170 - DNA, 51 bp, Artificial Sequence
  Synthetic Polynucleotide
ttagcctggtatcaagagaaacctgggaaaactaataagctccttatctac

SEQ ID NO: 171 - DNA, 9 bp, Artificial Sequence
  Synthetic Polynucleotide
tctggatcc

SEQ ID NO: 172 - DNA, 108 bp, Artificial Sequence
  Synthetic Polynucleotide
actttgcaatctggaattccatcaagattcaggggcagtggatctggtacagatttcactctcaccatcagtagcctggagcctgaagattttgcaatgtattactgt

SEQ ID NO: 173 - DNA, 27 bp, Artificial Sequence
  Synthetic Polynucleotide
caacagcatgatgaatccccgtacacg

SEQ ID NO: 174 - DNA, 33 bp, Artificial Sequence
  Synthetic Polynucleotide
ttcggagaggggaccaagctggaaataaaacgg

SEQ ID NO: 175 - DNA, 75 bp, Artificial Sequence
  Synthetic Polynucleotide
gaagtgcagctgttggagactgggggaggcttagtgcagcctggagggtcccggaaactctcctgtgcagcctct

SEQ ID NO: 176 - DNA, 24 bp, Artificial Sequence
  Synthetic Polynucleotide
ggattcactttcagtaactatgga

SEQ ID NO: 177 - DNA, 51 bp, Artificial Sequence
  Synthetic Polynucleotide
atgcactgggttcgtcaggctccagggaaggggctggagtgggtcgcacat

SEQ ID NO: 178 - DNA, 24 bp, Artificial Sequence
  Synthetic Polynucleotide
attagtcgttacagtgataccatc

SEQ ID NO: 179 - DNA, 114 bp, Artificial Sequence
  Synthetic Polynucleotide
tactatgcagacgcagtgaagggccgattcaccatctccagagacaatgccaagaacaccctgttcctgcaaatgaccactctaaggtctgaggacacggccatatattactgt

SEQ ID NO: 180 - DNA, 18 bp, Artificial Sequence
  Synthetic Polynucleotide
acaagtgctatggactac

SEQ ID NO: 181 - DNA, 33 bp, Artificial Sequence; identical to SEQ ID NO: 139
  Synthetic Polynucleotide

SEQ ID NO: 182 - DNA, 78 bp, Artificial Sequence
  Synthetic Polynucleotide
gatatccagatgacacagactacatcctccctgtctgcctctctgggagacagagtcaccatcagttgcagggcaagt

SEQ ID NO: 183 - DNA, 18 bp, Artificial Sequence
  Synthetic Polynucleotide
caggacattagcaattat

SEQ ID NO: 184 - DNA, 51 bp, Artificial Sequence
  Synthetic Polynucleotide
ttaaactggtttcagcagaaaccagatggaactattaaactcctgatctac

SEQ ID NO: 185 - DNA, 9 bp, Artificial Sequence
  Synthetic Polynucleotide
tacacatca

SEQ ID NO: 186 - DNA, 108 bp, Artificial Sequence
  Synthetic Polynucleotide
agattacactcaggagtcccatcaaggttcagtggcagtgggtctggaacagattattctctcaccattagcaacctggaacaagaagatattgccacttacttttgc

SEQ ID NO: 187 - DNA, 27 bp, Artificial Sequence
  Synthetic Polynucleotide
cagcagggtaatacgcttccattcacg

SEQ ID NO: 188 - DNA, 33 bp, Artificial Sequence
  Synthetic Polynucleotide
ttcggctcggggacaaagttggaaataaaacgg

SEQ ID NO: 189 - DNA, 75 bp, Artificial Sequence
  Synthetic Polynucleotide
gaggtgaagctggtggaatctgggggaggcttagtgcagcctggagggtcccggaaactctcctgtgcagcctct

SEQ ID NO: 190 - DNA, 24 bp, Artificial Sequence
  Synthetic Polynucleotide
ggattcactttcagtagctatgga

SEQ ID NO: 191 - DNA, 51 bp, Artificial Sequence
  Synthetic Polynucleotide
atgcactggattcgtcagactccagacaaggggctggaatgggtcgcatat

SEQ ID NO: 192 - DNA, 24 bp, Artificial Sequence
  Synthetic Polynucleotide
attagtagtaacagtgatcacatc

SEQ ID NO: 193 - DNA, 114 bp, Artificial Sequence
  Synthetic Polynucleotide
ttctatacagacacagtgaagggccgattcaccatctccagagacaatgccaagaacaccctgttcctgcaaatgaccagtctaaggtctgaggacacggccatgtattactgt

SEQ ID NO: 194 - DNA, 18 bp, Artificial Sequence
  Synthetic Polynucleotide
gcaagccgtatggactac

SEQ ID NO: 195 - DNA, 33 bp, Artificial Sequence; identical to SEQ ID NO: 139
  Synthetic Polynucleotide

SEQ ID NO: 196 - DNA, 78 bp, Artificial Sequence; identical to SEQ ID NO: 182
  Synthetic Polynucleotide

SEQ ID NO: 197 - DNA, 18 bp, Artificial Sequence
  Synthetic Polynucleotide
caggacattagcaatcat

SEQ ID NO: 198 - DNA, 51 bp, Artificial Sequence
  Synthetic Polynucleotide
ttaaactggtatcagcagaaaccagatggaactgttaaactcctgatctac

SEQ ID NO: 199 - DNA, 9 bp, Artificial Sequence
  Synthetic Polynucleotide
tacacatca

SEQ ID NO: 200 - DNA, 108 bp, Artificial Sequence
  Synthetic Polynucleotide
agattacactcaggagtcccatcaaggttcagtggcagtgggtctggaacagattattctctcaccattagcaacctggagcaaggcgatattgccacttacttttgc

SEQ ID NO: 201 - DNA, 27 bp, Artificial Sequence
  Synthetic Polynucleotide
caacagggtagtacgcttccgtacaca

SEQ ID NO: 202 - DNA, 33 bp, Artificial Sequence; identical to SEQ ID NO: 146
  Synthetic Polynucleotide

SEQ ID NO: 203 - DNA, 75 bp, Artificial Sequence
  Synthetic Polynucleotide
cagatccagttggtgcaatctggacctgagctgaagaagcctggagagacagtcaagatctcctgcaaggcttct

SEQ ID NO: 204 - DNA, 24 bp, Artificial Sequence
  Synthetic Polynucleotide
gggtataccttcacatactatcca

SEQ ID NO: 205 - DNA, 51 bp, Artificial Sequence
  Synthetic Polynucleotide
atccactgggtgaagcaggctccaggaaagggtttaaagtggatgggctgg

SEQ ID NO: 206 - DNA, 24 bp, Artificial Sequence
  Synthetic Polynucleotide
ataaacacctactctggagtgcca

SEQ ID NO: 207 - DNA, 114 bp, Artificial Sequence
  Synthetic Polynucleotide
acatatgcagatgacttcaagggacggtttgccttctctttggaaacctctgccaacactgcatatttgcagatcaacaacctcagaaatgaagacatggctacatatttctgt

SEQ ID NO: 208 - DNA, 30 bp, Artificial Sequence
  Synthetic Polynucleotide
gcaaggggggatagtagcctttttgactac

SEQ ID NO: 209 - DNA, 33 bp, Artificial Sequence
  Synthetic Polynucleotide
tggggccaaggcaccactctcacagtctcctca

SEQ ID NO: 210 - DNA, 78 bp, Artificial Sequence
  Synthetic Polynucleotide
ggacatcaaatgacccagtctccatcctccttatctgcctctctgggagaaagagtcagtctcacttgtcgggcaagt

SEQ ID NO: 211 - DNA, 18 bp, Artificial Sequence
  Synthetic Polynucleotide
caggaaattagtggttac

SEQ ID NO: 212 - DNA, 51 bp, Artificial Sequence
  Synthetic Polynucleotide
ttaatctggcttcagcagaaaccggatggaactattaaacgcctgatctac

SEQ ID NO: 213 - DNA, 9 bp, Artificial Sequence
  Synthetic Polynucleotide
gccgcatcc

SEQ ID NO: 214 - DNA, 108 bp, Artificial Sequence
  Synthetic Polynucleotide
actttaaattctggtgtcccaaaaaggttcagtggcagtaggtctgggtcagattactctctcaccatcagcagccttgagtctgaagattttgctgactattactgt

SEQ ID NO: 215 - DNA, 27 bp, Artificial Sequence
  Synthetic Polynucleotide
ctacaatatactagttatcctctcacg

SEQ ID NO: 216 - DNA, 33 bp, Artificial Sequence; identical to SEQ ID NO: 160
  Synthetic Polynucleotide

SEQ ID NO: 217 - DNA, 75 bp, Artificial Sequence; identical to SEQ ID NO: 203
  Synthetic Polynucleotide

SEQ ID NO: 218 - DNA, 24 bp, Artificial Sequence
  Synthetic Polynucleotide
ggatataccttcacaaactatcca

SEQ ID NO: 219 - DNA, 51 bp, Artificial Sequence
  Synthetic Polynucleotide
atacactgggtgaagcaggctccaggaaagggtttaaagtggatgggctgg

SEQ ID NO: 220 - DNA, 24 bp, Artificial Sequence
  Synthetic Polynucleotide
ataaacacctactctggagtgcca

SEQ ID NO: 221 - DNA, 114 bp, Artificial Sequence
  Synthetic Polynucleotide
acatatgcagatgacttcaagggacggtttgccttctctttggaaacctctgccaacactgcatatttgcagctcaacaacctcagaaatgaggacatggctacatatttctgt

SEQ ID NO: 222 - DNA, 30 bp, Artificial Sequence; identical to SEQ ID NO: 208
  Synthetic Polynucleotide

SEQ ID NO: 223 - DNA, 33 bp, Artificial Sequence; identical to SEQ ID NO: 209
  Synthetic Polynucleotide

SEQ ID NO: 224 - DNA, 78 bp, Artificial Sequence
  Synthetic Polynucleotide
gacgttcagatgatccagtctccatcctccttatctgcctctctgggagaaagagtcagtctcacttgtcgggcaagt

SEQ ID NO: 225 - DNA, 18 bp, Artificial Sequence
  Synthetic Polynucleotide
caggaaattagtggttac

SEQ ID NO: 226 - DNA, 51 bp, Artificial Sequence
  Synthetic Polynucleotide
ttaatctggcttcagcagaaaccagatggaactattacacgcctgatctac

SEQ ID NO: 227 - DNA, 9 bp, Artificial Sequence
  Synthetic Polynucleotide
gccgcatcc

SEQ ID NO: 228 - DNA, 108 bp, Artificial Sequence
  Synthetic Polynucleotide
actttagattctggtgtcccaaaaaggttcagtggcagtaggtctgggtcagattactctctcaccatcagcagccttgagtctgaagattttgctgactattactgt

SEQ ID NO: 229 - DNA, 27 bp, Artificial Sequence
  Synthetic Polynucleotide
ctacaatatactagttatcctctcacg

SEQ ID NO: 230 - DNA, 33 bp, Artificial Sequence; identical to SEQ ID NO: 160
  Synthetic Polynucleotide